SEQUENCE LISTING (99 sequences)

SEQ ID NO: 1 - protein, 465 aa, Homo sapiens
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 2 - protein, 437 aa, Homo sapiens
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 3 - protein, 218 aa, Homo sapiens
MNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVVCLLPGM

SEQ ID NO: 4 - protein, 274 aa, Homo sapiens
MSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 5 - protein, 387 aa, Homo sapiens
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVGTGDTVVLRGALGGFRRGVAGCPNSLIKELHHFRILGEEQYNRYQQYGAEECVLQMGGVLCPRPGCGAGLLPEPDQRKVTCEGGNGLGCGYGQRRTK

SEQ ID NO: 6 - protein, 316 aa, Homo sapiens
MIVFVRFNSSHGFPVEVDSDTSIFQLKEVVAKRQGVPADQLRVIFAGKELRNDWTVQEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 7 - protein, 387 aa, Homo sapiens
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 8 - protein, 415 aa, Homo sapiens
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 9 - DNA, 1398 bp, Homo sapiens
atgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcgcctatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaaggatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgtgccgaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtag

SEQ ID NO: 10 - DNA, 1398 bp, Artificial Sequence
  Codon optimized polynucleotide
atgattgtgtttgtccggttcaactcctcccatggtttcccggtggaagtggactcagacaccagcatcttccaactgaaggaagtggtggccaagcgtcagggggtcccggcagaccagttgagagtgatcttcgctggaaaggaactgagaaacgactggactgtgcagaactgtgacctggaccaacagtccattgtgcacattgtccagcggccttggcggaaaggtcaagagatgaacgccactggtggagatgaccccaggaatgcagctgggggctgtgaacgggaacctcagagcctgaccagagtggacctcagctcctctgtcctcccgggagactccgtgggactggcagtcattctgcacactgacagccgcaaggattccccccctgcgggttcaccagctggacggtccatctacaactccgcctatgtgtactgcaagggaccctgccagagggtgcagccgggaaagctcagagtgcagtgcagcacttgcagacaagccaccctgaccctgacccagggcccatcctgctgggatgatgtcctgatccccaaccggatgtcaggggaatgccaaagccctcactgccctggaacctcggccgagttcttcttcaaatgtggagcccaccccacctcggacaaggaaacctcggtggcccttcacctcattgccaccaactcccgcaacatcacctgtatcacttgcactgatgttcgctctccggtgctggtgttccagtgcaactcccgacacgtgatctgcctggactgcttccacctgtactgtgtgaccagactgaaggacaggcagtttgtccacgacccccaactgggctactccttgccttgtgtggctggctgccccaactccctgatcaaggagttgcaccacttccggatcctgggagaggaacagtacaacagataccagcagtacggggcagaggaatgtgtcctccaaatggggggagtgctgtgcccccggcctggttgtggagctggcctcctgccggaacctgaccagcggaaggtcacttgcgagggtggaaacggcctgggctgtggcttcgccttctgtcgggagtgcaaggaggcctaccacgaaggagaatgctccgcggtgtttgaagcctcagggaccaccacacaagcctacagagtggatgagagggcagcggagcaggcccgcgccgaagcggcctccaaggagactatcaagaaaaccaccaagccatgccctaggtgccatgtgcctgtggaaaagaatggaggctgcatgcacatgaagtgcccccagccacagtgccgccttgaatggtgctggaactgtggctgcgagtggaacagagtgtgtatgggggaccactggtttgatgtgtga

SEQ ID NO: 11 - protein, 465 aa, Artificial Sequence
  Made in Lab - mutated parkin protein
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSAYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLKDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARAEAASKETIKKTTKPCPRCHVPVEKNGGCMHMKCPQPQCRLEWCWNCGCEWNRVCMGDHWFDV

SEQ ID NO: 12 - protein, 465 aa, Artificial Sequence
  Made in Lab - mutated Parkin protein
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLKDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARAEAASKETIKKTTKPCPRCHVPVEKNGGCMHMKCPQPQCRLEWCWNCGCEWNRVSMGDHWFDV

SEQ ID NO: 13 - protein, 465 aa, Artificial Sequence
  Made in Lab - mutated Parkin protein
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLKDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARAEAASKETIKKTTKPCPRCHVPVEKNGGCMHMKCPQPQCRLEWCWNCGCEWNRVCMGDHWADV

SEQ ID NO: 14 - protein, 465 aa, Artificial Sequence
  Made in Lab - mutated Parkin protein
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSAYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLKDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARAEAASKETIKKTTKPCPRCHVPVEKNGGCMHMKCPQPQCRLEWCWNCGCEWNRVSMGDHWFDV

SEQ ID NO: 15 - protein, 465 aa, Artificial Sequence
  Made in Lab - mutated Parkin protein
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSAYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLKDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARAEAASKETIKKTTKPCPRCHVPVEKNGGCMHMKCPQPQCRLEWCWNCGCEWNRVSMGDHWADV

SEQ ID NO: 16 - protein, 390 aa, Homo sapiens
KGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 17 - protein, 325 aa, Homo sapiens
SIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 18 - protein, 390 aa, Artificial Sequence
  Made in Lab - mutated Parkin protein
KGQEMNATGGDDPRNAAGGCEREPQSLTRVDLSSSVLPGDSVGLAVILHTDSRKDSPPAGSPAGRSIYNSAYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARAEAASKETIKKTTKPCPRCHVPVEKNGGCMHMKCPQPQCRLEWCWNCGCEWNRVCMGDHWFDV

SEQ ID NO: 19 - protein, 390 aa, Artificial Sequence
  Made in Lab - mutated Parkin protein
KGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLKDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARAEAASKETIKKTTKPCPRCHVPVEKNGGCMHMKCPQPQCRLEWCWNCGCEWNRVCMGDHWADV

SEQ ID NO: 20 - protein, 390 aa, Artificial Sequence
  Made in lab - mutated Parkin protein
KGQEMNATGGDDPRNAAGGCEREPQSLTRVDLSSSVLPGDSVGLAVILHTDSRKDSPPAGSPAGRSIYNSAYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLKDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARAEAASKETIKKTTKPCPRCHVPVEKNGGCMHMKCPQPQCRLEWCWNCGCEWNRVSMGDHWFDV

SEQ ID NO: 21 - protein, 390 aa, Artificial Sequence
  Made in Lab - mutated Parkin protein
KGQEMNATGGDDPRNAAGGCEREPQSLTRVDLSSSVLPGDSVGLAVILHTDSRKDSPPAGSPAGRSIYNSAYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLKDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARAEAASKETIKKTTKPCPRCHVPVEKNGGCMHMKCPQPQCRLEWCWNCGCEWNRVSMGDHWADV

SEQ ID NO: 22 - DNA, 1176 bp, Homo sapiens
atgaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcgcctatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgtgccgaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtag

SEQ ID NO: 23 - DNA, 979 bp, Homo sapiens
atgaatctacaacagcgcctatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgtgccgaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtag

SEQ ID NO: 24 - DNA, 1176 bp, Artificial Sequence
  Made in Lab - Codon optimized Parkin fragment polynucleotide
atgaaaggtcaagagatgaacgccactggtggagatgaccccaggaatgcagctgggggctgtgaacgggaacctcagagcctgaccagagtggacctcagctcctctgtcctcccgggagactccgtgggactggcagtcattctgcacactgacagccgcaaggattccccccctgcgggttcaccagctggacggtccatctacaactccgcctatgtgtactgcaagggaccctgccagagggtgcagccgggaaagctcagagtgcagtgcagcacttgcagacaagccaccctgaccctgacccagggcccatcctgctgggatgatgtcctgatccccaaccggatgtcaggggaatgccaaagccctcactgccctggaacctcggccgagttcttcttcaaatgtggagcccaccccacctcggacaaggaaacctcggtggcccttcacctcattgccaccaactcccgcaacatcacctgtatcacttgcactgatgttcgctctccggtgctggtgttccagtgcaactcccgacacgtgatctgcctggactgcttccacctgtactgtgtgaccagactgaatgacaggcagtttgtccacgacccccaactgggctactccttgccttgtgtggctggctgccccaactccctgatcaaggagttgcaccacttccggatcctgggagaggaacagtacaacagataccagcagtacggggcagaggaatgtgtcctccaaatggggggagtgctgtgcccccggcctggttgtggagctggcctcctgccggaacctgaccagcggaaggtcacttgcgagggtggaaacggcctgggctgtggcttcgccttctgtcgggagtgcaaggaggcctaccacgaaggagaatgctccgcggtgtttgaagcctcagggaccaccacacaagcctacagagtggatgagagggcagcggagcaggcccgcgccgaagcggcctccaaggagactatcaagaaaaccaccaagccatgccctaggtgccatgtgcctgtggaaaagaatggaggctgcatgcacatgaagtgcccccagccacagtgccgccttgaatggtgctggaactgtggctgcgagtggaacagagtgtgtatgggggaccactggtttgatgtgtga

SEQ ID NO: 25 - DNA, 979 bp, Homo sapiens
atgcatctacaactccgcctatgtgtactgcaagggaccctgccagagggtgcagccgggaaagctcagagtgcagtgcagcacttgcagacaagccaccctgaccctgacccagggcccatcctgctgggatgatgtcctgatccccaaccggatgtcaggggaatgccaaagccctcactgccctggaacctcggccgagttcttcttcaaatgtggagcccaccccacctcggacaaggaaacctcggtggcccttcacctcattgccaccaactcccgcaacatcacctgtatcacttgcactgatgttcgctctccggtgctggtgttccagtgcaactcccgacacgtgatctgcctggactgcttccacctgtactgtgtgaccagactgaatgacaggcagtttgtccacgacccccaactgggctactccttgccttgtgtggctggctgccccaactccctgatcaaggagttgcaccacttccggatcctgggagaggaacagtacaacagataccagcagtacggggcagaggaatgtgtcctccaaatggggggagtgctgtgcccccggcctggttgtggagctggcctcctgccggaacctgaccagcggaaggtcacttgcgagggtggaaacggcctgggctgtggcttcgccttctgtcgggagtgcaaggaggcctaccacgaaggagaatgctccgcggtgtttgaagcctcagggaccaccacacaagcctacagagtggatgagagggcagcggagcaggcccgcgccgaagcggcctccaaggagactatcaagaaaaccaccaagccatgccctaggtgccatgtgcctgtggaaaagaatggaggctgcatgcacatgaagtgcccccagccacagtgccgccttgaatggtgctggaactgtggctgcgagtggaacagagtgtgtatgggggaccactggtttgatgtgtga

SEQ ID NO: 26 - DNA, 168 bp, Adeno-associated virus
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgta

SEQ ID NO: 27 - DNA, 168 bp, Adeno-associated virus
tacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 28 - DNA, 822 bp, Artificial Sequence
  Made in Lab
gttacccaggctggagtgcagtggcacatttctgctcactgcaacctcctcctccctgggttcaagcaattctcctgcctcagcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattatgttgaaaacatagggtctggggaaaaagggatttaaaataagaagaaaaagaagacttgggacttaaaaagtcttttagaggccagctcaccaacattcagaacacccagtctgtgttgcacaatatgttacttaggtataaatcaaggattcatgtaattttgtcattccttgcgtgatattttaaaaaacattctgtgtaaggtatttataaagctctcttctaaaaatacaaaaatttgtggggccttgtagtcccagctacttgggaggctgaggcaggaggatttcttgaactgggaggcagagcttgcagtgagcccactgcatcag

SEQ ID NO: 29 - DNA, 253 bp, Homo sapiens
gcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagtc

SEQ ID NO: 30 - DNA, 281 bp, Gallus gallus
ggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggga

SEQ ID NO: 31 - DNA, 220 bp, Human betaherpesvirus 5
tggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccg

SEQ ID NO: 32 - DNA, 455 bp, Homo sapiens
caacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaatagcatgcgtgaggctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgtt

SEQ ID NO: 33 - DNA, 422 bp, Homo sapiens
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctc

SEQ ID NO: 34 - DNA, 281 bp, Homo sapiens
acttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgc

SEQ ID NO: 35 - DNA, 293 bp, Human betaherpesvirus 5
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattacca

SEQ ID NO: 36 - DNA, 953 bp, Homo sapiens
cgcgtccgcccgcgagcacagagcctcgcctttgccgatccgccgcccgtccacacccgccgccaggtaagcccggccagccgaccggggcatgcggccgcggcccttcgcccgtgcagagccgccgtctgggccgcagcggggggcgcatggggcggaaccggaccgccgtggggggcgcgggagaagcccctgggcctccggagatgggggacaccccacgccagttcgcaggcgcgaggccgcgctcgggcgggcgcgctccgggggtgccgctctcggggcgggggcaaccggcggggtctttgtctgagccgggctcttgccaatggggatcgcacggtgggcgcggcgtagcccccgtcaggcccggtgggggctggggcgccatgcgcgtgcgcgctggtcctttgggcgctaactgcgtgcgcgctgggaattggcgctaattgcgcgtgcgcgctgggactcaatggcgctaatcgcgcgtgcgttctggggcccgggcgcttgcgccacttcctgcccgagccgctggcgcccgagggtgtggccgctgcgtgcgcgcgcgcgacccggtcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcggcagcggctgctcttggcggccccgaggtgactatagccttcttttgtgtcttgatagttcgccagcctctgctaaccatgttcatgccttcttctttttcctacagctcctgggcaacgtgctggttattgtgctgtctcatcattttggcaaagaattc

SEQ ID NO: 37 - DNA, 1068 bp, Artificial Sequence
  Made in Lab - Chicken beta-actin exon/intron and rabbit globin intron
gtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 38 - DNA, 126 bp, Homo sapiens
agtctgcggtgggcagcggaggagtcgtgtcgtgcctgagagcgcagctgtgctcctgggcaccgcgcagtccgcccccgcggctcctggccagaccacccctaggaccccctgccccaagtcgca

SEQ ID NO: 39 - DNA, 121 bp, Human betaherpesvirus 5
tcagatcgcctggagaggccatccacgctgttttgacctccatagtggacaccgggaccgatccagcctccgcggccgggaacggtgcattggaacgcggattccccgtgccaagagtgac

SEQ ID NO: 40 - DNA, 512 bp, Artificial Sequence
  Made in Lab - adenovirus derived enhancer element
ctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccag

SEQ ID NO: 41 - DNA, 956 bp, Homo sapiens
ctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacgtgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttggcctttttgagtttggatcttggttcattctcaagcctcagacagtggttcaaagtttttttcttccatttcag

SEQ ID NO: 42 - DNA, 83 bp, Homo sapiens
tcagaagccccgggctcgtcagtcaaaccggttctctgtttgcactcggcagcacgggcaggcaagtggtccctaggttcggg

SEQ ID NO: 43 - DNA, 755 bp, Artificial Sequence
  Made in Lab - mutated woodchuck hepatitis regulatory element
ttcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggac

SEQ ID NO: 44 - DNA, 12 bp, Artificial Sequence
  CAAX motif
tgtgtgataatg

SEQ ID NO: 45 - DNA, 810 bp, Homo sapiens
ctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagtta

SEQ ID NO: 46 - DNA, 726 bp, Hepatitis B virus
ataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatc

SEQ ID NO: 47 - DNA, 755 bp, Artificial Sequence
  Made in Lab - HepB derived enhancer element
ttcctgtaaacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagctggactggagcaaacctcatcgggaccgacaattctgtcgtactctcccgcaagcactcaccgtttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggac

SEQ ID NO: 48 - DNA, 94 bp, Homo sapiens
gctggagcctcggtagccgttcctcctgcccgctgggcctcccaacgggccctcctcccctccttgcaccggcccttcctggtctttgaataaa

SEQ ID NO: 49 - DNA, 596 bp, Woodchuck hepatitis virus
attcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcc

SEQ ID NO: 50 - DNA, 387 bp, Oryctolagus cuniculus
tggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 51 - DNA, 251 bp, Bos taurus
ttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaatacaatagcaggcatgctggggatgcggtgggctctatgggtacccaggtgctgaagaattgacccggttcctcctggg

SEQ ID NO: 52 - DNA, 202 bp, Homo sapiens
ctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 53 - DNA, 4523 bp, Artificial Sequence
  Made in Lab - Expression cassette
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcaggggaattcaaagcaagccgccaccatgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcgcctatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaaggatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgtgccgaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtagtaatggattcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggacgctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttcaagcaattctcctgcctcagcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattatgttgaaaacatagggtctggggaaaaagggatttaaaataagaagaaaaagaagacttgggacttaaaaagtcttttagaggccagctcaccaacattcagaacacccagtctgtgttgcacaatatgttacttaggtataaatcaaggattcatgtaattttgtcattccttgcgtgatattttaaaaaacattctgtgtaaggtatttataaagctctcttctaaaaatacaaaaatttgtggggccttgtagtcccagctacttgggaggctgaggcaggaggatttcttgaactgggaggcagagcttgcagtgagcccactgcatcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggcc

SEQ ID NO: 54 - DNA, 4523 bp, Artificial Sequence
  Made in Lab - Expression cassette
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcaggggaattcaaagcaagccgccaccatgattgtgtttgtccggttcaactcctcccatggtttcccggtggaagtggactcagacaccagcatcttccaactgaaggaagtggtggccaagcgtcagggggtcccggcagaccagttgagagtgatcttcgctggaaaggaactgagaaacgactggactgtgcagaactgtgacctggaccaacagtccattgtgcacattgtccagcggccttggcggaaaggtcaagagatgaacgccactggtggagatgaccccaggaatgcagctgggggctgtgaacgggaacctcagagcctgaccagagtggacctcagctcctctgtcctcccgggagactccgtgggactggcagtcattctgcacactgacagccgcaaggattccccccctgcgggttcaccagctggacggtccatctacaactccgcctatgtgtactgcaagggaccctgccagagggtgcagccgggaaagctcagagtgcagtgcagcacttgcagacaagccaccctgaccctgacccagggcccatcctgctgggatgatgtcctgatccccaaccggatgtcaggggaatgccaaagccctcactgccctggaacctcggccgagttcttcttcaaatgtggagcccaccccacctcggacaaggaaacctcggtggcccttcacctcattgccaccaactcccgcaacatcacctgtatcacttgcactgatgttcgctctccggtgctggtgttccagtgcaactcccgacacgtgatctgcctggactgcttccacctgtactgtgtgaccagactgaaggacaggcagtttgtccacgacccccaactgggctactccttgccttgtgtggctggctgccccaactccctgatcaaggagttgcaccacttccggatcctgggagaggaacagtacaacagataccagcagtacggggcagaggaatgtgtcctccaaatggggggagtgctgtgcccccggcctggttgtggagctggcctcctgccggaacctgaccagcggaaggtcacttgcgagggtggaaacggcctgggctgtggcttcgccttctgtcgggagtgcaaggaggcctaccacgaaggagaatgctccgcggtgtttgaagcctcagggaccaccacacaagcctacagagtggatgagagggcagcggagcaggcccgcgccgaagcggcctccaaggagactatcaagaaaaccaccaagccatgccctaggtgccatgtgcctgtggaaaagaatggaggctgcatgcacatgaagtgcccccagccacagtgccgccttgaatggtgctggaactgtggctgcgagtggaacagagtgtgtatgggggaccactggtttgatgtgtgataatggattcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggacgctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttcaagcaattctcctgcctcagcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattatgttgaaaacatagggtctggggaaaaagggatttaaaataagaagaaaaagaagacttgggacttaaaaagtcttttagaggccagctcaccaacattcagaacacccagtctgtgttgcacaatatgttacttaggtataaatcaaggattcatgtaattttgtcattccttgcgtgatattttaaaaaacattctgtgtaaggtatttataaagctctcttctaaaaatacaaaaatttgtggggccttgtagtcccagctacttgggaggctgaggcaggaggatttcttgaactgggaggcagagcttgcagtgagcccactgcatcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggcc

SEQ ID NO: 55 - DNA, 4301 bp, Artificial Sequence
  Made in Lab - Expression cassette
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcaggggaattcaaagcaagccgccaccatgaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcgcctatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgtgccgaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtagtaatggattcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggacgctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttcaagcaattctcctgcctcagcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattatgttgaaaacatagggtctggggaaaaagggatttaaaataagaagaaaaagaagacttgggacttaaaaagtcttttagaggccagctcaccaacattcagaacacccagtctgtgttgcacaatatgttacttaggtataaatcaaggattcatgtaattttgtcattccttgcgtgatattttaaaaaacattctgtgtaaggtatttataaagctctcttctaaaaatacaaaaatttgtggggccttgtagtcccagctacttgggaggctgaggcaggaggatttcttgaactgggaggcagagcttgcagtgagcccactgcatcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggcc

SEQ ID NO: 56 - DNA, 4301 bp, Artificial Sequene
  Made in Lab - Expression cassette
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcaggggaattcaaagcaagccgccaccatgaaaggtcaagagatgaacgccactggtggagatgaccccaggaatgcagctgggggctgtgaacgggaacctcagagcctgaccagagtggacctcagctcctctgtcctcccgggagactccgtgggactggcagtcattctgcacactgacagccgcaaggattccccccctgcgggttcaccagctggacggtccatctacaactccgcctatgtgtactgcaagggaccctgccagagggtgcagccgggaaagctcagagtgcagtgcagcacttgcagacaagccaccctgaccctgacccagggcccatcctgctgggatgatgtcctgatccccaaccggatgtcaggggaatgccaaagccctcactgccctggaacctcggccgagttcttcttcaaatgtggagcccaccccacctcggacaaggaaacctcggtggcccttcacctcattgccaccaactcccgcaacatcacctgtatcacttgcactgatgttcgctctccggtgctggtgttccagtgcaactcccgacacgtgatctgcctggactgcttccacctgtactgtgtgaccagactgaatgacaggcagtttgtccacgacccccaactgggctactccttgccttgtgtggctggctgccccaactccctgatcaaggagttgcaccacttccggatcctgggagaggaacagtacaacagataccagcagtacggggcagaggaatgtgtcctccaaatggggggagtgctgtgcccccggcctggttgtggagctggcctcctgccggaacctgaccagcggaaggtcacttgcgagggtggaaacggcctgggctgtggcttcgccttctgtcgggagtgcaaggaggcctaccacgaaggagaatgctccgcggtgtttgaagcctcagggaccaccacacaagcctacagagtggatgagagggcagcggagcaggcccgcgccgaagcggcctccaaggagactatcaagaaaaccaccaagccatgccctaggtgccatgtgcctgtggaaaagaatggaggctgcatgcacatgaagtgcccccagccacagtgccgccttgaatggtgctggaactgtggctgcgagtggaacagagtgtgtatgggggaccactggtttgatgtgtgataatggattcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggacgctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttcaagcaattctcctgcctcagcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattatgttgaaaacatagggtctggggaaaaagggatttaaaataagaagaaaaagaagacttgggacttaaaaagtcttttagaggccagctcaccaacattcagaacacccagtctgtgttgcacaatatgttacttaggtataaatcaaggattcatgtaattttgtcattccttgcgtgatattttaaaaaacattctgtgtaaggtatttataaagctctcttctaaaaatacaaaaatttgtggggccttgtagtcccagctacttgggaggctgaggcaggaggatttcttgaactgggaggcagagcttgcagtgagcccactgcatcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggcc

SEQ ID NO: 57 - DNA, 4104 bp, Artificial Sequence
  Made in Lab - Expression cassette
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcaggggaattcaaagcaagccgccaccatgaatctacaacagcgcctatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgtgccgaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtagtaatggattcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggacgctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttcaagcaattctcctgcctcagcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattatgttgaaaacatagggtctggggaaaaagggatttaaaataagaagaaaaagaagacttgggacttaaaaagtcttttagaggccagctcaccaacattcagaacacccagtctgtgttgcacaatatgttacttaggtataaatcaaggattcatgtaattttgtcattccttgcgtgatattttaaaaaacattctgtgtaaggtatttataaagctctcttctaaaaatacaaaaatttgtggggccttgtagtcccagctacttgggaggctgaggcaggaggatttcttgaactgggaggcagagcttgcagtgagcccactgcatcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggcc

SEQ ID NO: 58 - DNA, 4104 bp, Artificial Sequence
  Made in Lab - Expression cassette
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcaggggaattcaaagcaagccgccaccatgcatctacaactccgcctatgtgtactgcaagggaccctgccagagggtgcagccgggaaagctcagagtgcagtgcagcacttgcagacaagccaccctgaccctgacccagggcccatcctgctgggatgatgtcctgatccccaaccggatgtcaggggaatgccaaagccctcactgccctggaacctcggccgagttcttcttcaaatgtggagcccaccccacctcggacaaggaaacctcggtggcccttcacctcattgccaccaactcccgcaacatcacctgtatcacttgcactgatgttcgctctccggtgctggtgttccagtgcaactcccgacacgtgatctgcctggactgcttccacctgtactgtgtgaccagactgaatgacaggcagtttgtccacgacccccaactgggctactccttgccttgtgtggctggctgccccaactccctgatcaaggagttgcaccacttccggatcctgggagaggaacagtacaacagataccagcagtacggggcagaggaatgtgtcctccaaatggggggagtgctgtgcccccggcctggttgtggagctggcctcctgccggaacctgaccagcggaaggtcacttgcgagggtggaaacggcctgggctgtggcttcgccttctgtcgggagtgcaaggaggcctaccacgaaggagaatgctccgcggtgtttgaagcctcagggaccaccacacaagcctacagagtggatgagagggcagcggagcaggcccgcgccgaagcggcctccaaggagactatcaagaaaaccaccaagccatgccctaggtgccatgtgcctgtggaaaagaatggaggctgcatgcacatgaagtgcccccagccacagtgccgccttgaatggtgctggaactgtggctgcgagtggaacagagtgtgtatgggggaccactggtttgatgtgtgataatggattcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggacgctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttcaagcaattctcctgcctcagcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattatgttgaaaacatagggtctggggaaaaagggatttaaaataagaagaaaaagaagacttgggacttaaaaagtcttttagaggccagctcaccaacattcagaacacccagtctgtgttgcacaatatgttacttaggtataaatcaaggattcatgtaattttgtcattccttgcgtgatattttaaaaaacattctgtgtaaggtatttataaagctctcttctaaaaatacaaaaatttgtggggccttgtagtcccagctacttgggaggctgaggcaggaggatttcttgaactgggaggcagagcttgcagtgagcccactgcatcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggcc

SEQ ID NO: 59 - protein, 735 aa, Adeno-associated virus 2 [sequence omitted]

SEQ ID NO: 60 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 61 - protein, 743 aa, Artificial Sequence
  Synthetic construct - AAV9 variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 62 - protein, 7 aa, Artificial Sequence
  Peptide insert
TLAVPFK

SEQ ID NO: 63 - protein, 7 aa, Artificial Sequence
  Peptide insert
KFPVALT

SEQ ID NO: 64 - protein, 110 aa, Homo sapiens
MAVRQALGRGLQLGRALLLRFTGKPGRAYGLGRPGPAAGCVRGERPGWAAGPGAEPRRVGLGLPNRLRFFRQSVAGLAARLQRQFVVRAWGCAGPCGRAVFLAFGLGLGL

SEQ ID NO: 65 - protein, 94 aa, Homo sapiens
MAVRQALGRGLQLGRALLLRFTGKPGRAYGLGRPGPAAGCVRGERPGWAAGPGAEPRRVGLGLPNRLRFFRQSVAGLAARLQRQFVVRAWGCAG

SEQ ID NO: 66 - protein, 34 aa, Homo sapiens
MAVRQALGRGLQLGRALLLRFTGKPGRAYGLGRP

SEQ ID NO: 67 - protein, 16 aa, Homo sapiens
PCGRAVFLAFGLGLGL

SEQ ID NO: 68 - protein, 16 aa, Artificial Sequence
  Made in Lab - muted PINK1 protein
PCGRAVFLAMGLGLGL

SEQ ID NO: 69 - protein, 16 aa, Artificial Sequence
  Made in Lab - muted PINK1 protein
PCGRAVFLAAGLGLGL

SEQ ID NO: 70 - protein, 110 aa, Homo sapiens; identical to SEQ ID NO: 64

SEQ ID NO: 71 - protein, 110 aa, Artificial Sequence
  Made in Lab - muted PINK1 protein
MAVRQALGRGLQLGRALLLRFTGKPGRAYGLGRPGPAAGCVRGERPGWAAGPGAEPRRVGLGLPNRLRFFRQSVAGLAARLQRQFVVRAWGCAGPCGRAVFLAMGLGLGL

SEQ ID NO: 72 - protein, 110 aa, Artificial Sequence
  Made in Lab - muted PINK1 protein
MAVRQALGRGLQLGRALLLRFTGKPGRAYGLGRPGPAAGCVRGERPGWAAGPGAEPRRVGLGLPNRLRFFRQSVAGLAARLQRQFVVRAWGCAGPCGRAVFLAAGLGLGL

SEQ ID NO: 73 - protein, 325 aa, Artificial Sequence
  Made in Lab - mutated Parkin protein
SIYNSAYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARAEAASKETIKKTTKPCPRCHVPVEKNGGCMHMKCPQPQCRLEWCWNCGCEWNRVCMGDHWFDV

SEQ ID NO: 74 - protein, 390 aa, Artificial Sequence; identical to SEQ ID NO: 18
  Made in Lab - mutated Parkin protein

SEQ ID NO: 75 - protein, 500 aa, Artificial Sequence
  Made in Lab - fusion protein
MAVRQALGRGLQLGRALLLRFTGKPGRAYGLGRPGPAAGCVRGERPGWAAGPGAEPRRVGLGLPNRLRFFRQSVAGLAARLQRQFVVRAWGCAGPCGRAVFLAFGLGLGLKGQEMNATGGDDPRNAAGGCEREPQSLTRVDLSSSVLPGDSVGLAVILHTDSRKDSPPAGSPAGRSIYNSAYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARAEAASKETIKKTTKPCPRCHVPVEKNGGCMHMKCPQPQCRLEWCWNCGCEWNRVCMGDHWFDV

SEQ ID NO: 76 - protein, 435 aa, Artificial Sequence
  Made in Lab - fusion protein
MAVRQALGRGLQLGRALLLRFTGKPGRAYGLGRPGPAAGCVRGERPGWAAGPGAEPRRVGLGLPNRLRFFRQSVAGLAARLQRQFVVRAWGCAGPCGRAVFLAFGLGLGLSIYNSAYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARAEAASKETIKKTTKPCPRCHVPVEKNGGCMHMKCPQPQCRLEWCWNCGCEWNRVCMGDHWFDV

SEQ ID NO: 77 - DNA, 1503 bp, Artificial Sequence
  Made in Lab - polynucleotide encoding fusion protein
atggcggtgcgacaggcgctgggccgcggcctgcagctgggtcgagcgctgctgctgcgcttcacgggcaagcccggccgggcctacggcttggggcggccgggcccggcggcgggctgtgtccgcggggagcgtccaggctgggccgcaggaccgggcgcggagcctcgcagggtcgggctcgggctccctaaccgtctccgcttcttccgccagtcggtggccgggctggcggcgcggttgcagcggcagttcgtggtgcgggcctggggctgcgcgggcccttgcggccgggcagtctttctggccttcgggctagggctgggcctcaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcgcctatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgtgccgaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtag

SEQ ID NO: 78 - DNA, 1503 bp, Artificial Sequence
  Made in Lab - codon optimized polynucleotide encoding fusion protein
atggctgtgcggcaggcactgggtcggggattgcaattgggaagagcattgcttctcagatttactggaaaaccaggaagagcatacgggttgggtcgccctggtcccgccgctgggtgcgtacgaggtgaacggccaggttgggctgcgggcccgggtgctgagccaagaagagtagggttgggattgcccaatcgccttaggttcttccggcagagcgtggctggtttggcagctagactccagaggcagtttgtagtgagggcctggggctgtgcaggcccatgcggacgagccgtatttttggctttcggacttgggttgggcctcaaaggtcaagagatgaacgccactggtggagatgaccccaggaatgcagctgggggctgtgaacgggaacctcagagcctgaccagagtggacctcagctcctctgtcctcccgggagactccgtgggactggcagtcattctgcacactgacagccgcaaggattccccccctgcgggttcaccagctggacggtccatctacaactccgcctatgtgtactgcaagggaccctgccagagggtgcagccgggaaagctcagagtgcagtgcagcacttgcagacaagccaccctgaccctgacccagggcccatcctgctgggatgatgtcctgatccccaaccggatgtcaggggaatgccaaagccctcactgccctggaacctcggccgagttcttcttcaaatgtggagcccaccccacctcggacaaggaaacctcggtggcccttcacctcattgccaccaactcccgcaacatcacctgtatcacttgcactgatgttcgctctccggtgctggtgttccagtgcaactcccgacacgtgatctgcctggactgcttccacctgtactgtgtgaccagactgaatgacaggcagtttgtccacgacccccaactgggctactccttgccttgtgtggctggctgccccaactccctgatcaaggagttgcaccacttccggatcctgggagaggaacagtacaacagataccagcagtacggggcagaggaatgtgtcctccaaatggggggagtgctgtgcccccggcctggttgtggagctggcctcctgccggaacctgaccagcggaaggtcacttgcgagggtggaaacggcctgggctgtggcttcgccttctgtcgggagtgcaaggaggcctaccacgaaggagaatgctccgcggtgtttgaagcctcagggaccaccacacaagcctacagagtggatgagagggcagcggagcaggcccgcgccgaagcggcctccaaggagactatcaagaaaaccaccaagccatgccctaggtgccatgtgcctgtggaaaagaatggaggctgcatgcacatgaagtgcccccagccacagtgccgccttgaatggtgctggaactgtggctgcgagtggaacagagtgtgtatgggggaccactggtttgatgtgtga

SEQ ID NO: 79 - DNA, 1306 bp, Artificial Sequence
  Made in Lab - polynucleotide encoding fusion protein
atggcggtgcgacaggcgctgggccgcggcctgcagctgggtcgagcgctgctgctgcgcttcacgggcaagcccggccgggcctacggcttggggcggccgggcccggcggcgggctgtgtccgcggggagcgtccaggctgggccgcaggaccgggcgcggagcctcgcagggtcgggctcgggctccctaaccgtctccgcttcttccgccagtcggtggccgggctggcggcgcggttgcagcggcagttcgtggtgcgggcctggggctgcgcgggcccttgcggccgggcagtctttctggccttcgggctagggctgggcctcaatctacaacagcgcctatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgtgccgaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtag

SEQ ID NO: 80 - DNA, 1306 bp, Artificial Sequence
  Made in Lab - codon optimized polynucleotide encoding fusion protein
atggctgtgcggcaggcactgggtcggggattgcaattgggaagagcattgcttctcagatttactggaaaaccaggaagagcatacgggttgggtcgccctggtcccgccgctgggtgcgtacgaggtgaacggccaggttgggctgcgggcccgggtgctgagccaagaagagtagggttgggattgcccaatcgccttaggttcttccggcagagcgtggctggtttggcagctagactccagaggcagtttgtagtgagggcctggggctgtgcaggcccatgcggacgagccgtatttttggctttcggacttgggttgggcctccatctacaactccgcctatgtgtactgcaagggaccctgccagagggtgcagccgggaaagctcagagtgcagtgcagcacttgcagacaagccaccctgaccctgacccagggcccatcctgctgggatgatgtcctgatccccaaccggatgtcaggggaatgccaaagccctcactgccctggaacctcggccgagttcttcttcaaatgtggagcccaccccacctcggacaaggaaacctcggtggcccttcacctcattgccaccaactcccgcaacatcacctgtatcacttgcactgatgttcgctctccggtgctggtgttccagtgcaactcccgacacgtgatctgcctggactgcttccacctgtactgtgtgaccagactgaatgacaggcagtttgtccacgacccccaactgggctactccttgccttgtgtggctggctgccccaactccctgatcaaggagttgcaccacttccggatcctgggagaggaacagtacaacagataccagcagtacggggcagaggaatgtgtcctccaaatggggggagtgctgtgcccccggcctggttgtggagctggcctcctgccggaacctgaccagcggaaggtcacttgcgagggtggaaacggcctgggctgtggcttcgccttctgtcgggagtgcaaggaggcctaccacgaaggagaatgctccgcggtgtttgaagcctcagggaccaccacacaagcctacagagtggatgagagggcagcggagcaggcccgcgccgaagcggcctccaaggagactatcaagaaaaccaccaagccatgccctaggtgccatgtgcctgtggaaaagaatggaggctgcatgcacatgaagtgcccccagccacagtgccgccttgaatggtgctggaactgtggctgcgagtggaacagagtgtgtatgggggaccactggtttgatgtgtga

SEQ ID NO: 81 - DNA, 1503 bp, Artificial Sequence
  Made in Lab - polynucleotide encoding fusion protein
atggcggtgcgacaggcgctgggccgcggcctgcagctgggtcgagcgctgctgctgcgcttcacgggcaagcccggccgggcctacggcttggggcggccgggcccggcggcgggctgtgtccgcggggagcgtccaggctgggccgcaggaccgggcgcggagcctcgcagggtcgggctcgggctccctaaccgtctccgcttcttccgccagtcggtggccgggctggcggcgcggttgcagcggcagttcgtggtgcgggcctggggctgcgcgggcccttgcggccgggcagtctttctggccatggggctagggctgggcctcaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcgcctatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgtgccgaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtag

SEQ ID NO: 82 - DNA, 1503 bp, Artificial Sequence
  Made in Lab - codon optimized polynucleotide encoding fusion protein
atggctgtgcggcaggcactgggtcggggattgcaattgggaagagcattgcttctcagatttactggaaaaccaggaagagcatacgggttgggtcgccctggtcccgccgctgggtgcgtacgaggtgaacggccaggttgggctgcgggcccgggtgctgagccaagaagagtagggttgggattgcccaatcgccttaggttcttccggcagagcgtggctggtttggcagctagactccagaggcagtttgtagtgagggcctggggctgtgcaggcccatgcggacgagccgtatttttggctatgggacttgggttgggcctcaaaggtcaagagatgaacgccactggtggagatgaccccaggaatgcagctgggggctgtgaacgggaacctcagagcctgaccagagtggacctcagctcctctgtcctcccgggagactccgtgggactggcagtcattctgcacactgacagccgcaaggattccccccctgcgggttcaccagctggacggtccatctacaactccgcctatgtgtactgcaagggaccctgccagagggtgcagccgggaaagctcagagtgcagtgcagcacttgcagacaagccaccctgaccctgacccagggcccatcctgctgggatgatgtcctgatccccaaccggatgtcaggggaatgccaaagccctcactgccctggaacctcggccgagttcttcttcaaatgtggagcccaccccacctcggacaaggaaacctcggtggcccttcacctcattgccaccaactcccgcaacatcacctgtatcacttgcactgatgttcgctctccggtgctggtgttccagtgcaactcccgacacgtgatctgcctggactgcttccacctgtactgtgtgaccagactgaatgacaggcagtttgtccacgacccccaactgggctactccttgccttgtgtggctggctgccccaactccctgatcaaggagttgcaccacttccggatcctgggagaggaacagtacaacagataccagcagtacggggcagaggaatgtgtcctccaaatggggggagtgctgtgcccccggcctggttgtggagctggcctcctgccggaacctgaccagcggaaggtcacttgcgagggtggaaacggcctgggctgtggcttcgccttctgtcgggagtgcaaggaggcctaccacgaaggagaatgctccgcggtgtttgaagcctcagggaccaccacacaagcctacagagtggatgagagggcagcggagcaggcccgcgccgaagcggcctccaaggagactatcaagaaaaccaccaagccatgccctaggtgccatgtgcctgtggaaaagaatggaggctgcatgcacatgaagtgcccccagccacagtgccgccttgaatggtgctggaactgtggctgcgagtggaacagagtgtgtatgggggaccactggtttgatgtgtga

SEQ ID NO: 83 - DNA, 4628 bp, Artificial Sequence
  Made in Lab - Expression cassette
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcaggggaattcaaagcaagccgccaccatggcggtgcgacaggcgctgggccgcggcctgcagctgggtcgagcgctgctgctgcgcttcacgggcaagcccggccgggcctacggcttggggcggccgggcccggcggcgggctgtgtccgcggggagcgtccaggctgggccgcaggaccgggcgcggagcctcgcagggtcgggctcgggctccctaaccgtctccgcttcttccgccagtcggtggccgggctggcggcgcggttgcagcggcagttcgtggtgcgggcctggggctgcgcgggcccttgcggccgggcagtctttctggccttcgggctagggctgggcctcaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcgcctatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgtgccgaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtagtaatggattcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggacgctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttcaagcaattctcctgcctcagcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattatgttgaaaacatagggtctggggaaaaagggatttaaaataagaagaaaaagaagacttgggacttaaaaagtcttttagaggccagctcaccaacattcagaacacccagtctgtgttgcacaatatgttacttaggtataaatcaaggattcatgtaattttgtcattccttgcgtgatattttaaaaaacattctgtgtaaggtatttataaagctctcttctaaaaatacaaaaatttgtggggccttgtagtcccagctacttgggaggctgaggcaggaggatttcttgaactgggaggcagagcttgcagtgagcccactgcatcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggcc

SEQ ID NO: 84 - DNA, 4628 bp, Artificial Sequence
  Made in Lab - Expression cassette
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcaggggaattcaaagcaagccgccaccatggctgtgcggcaggcactgggtcggggattgcaattgggaagagcattgcttctcagatttactggaaaaccaggaagagcatacgggttgggtcgccctggtcccgccgctgggtgcgtacgaggtgaacggccaggttgggctgcgggcccgggtgctgagccaagaagagtagggttgggattgcccaatcgccttaggttcttccggcagagcgtggctggtttggcagctagactccagaggcagtttgtagtgagggcctggggctgtgcaggcccatgcggacgagccgtatttttggctttcggacttgggttgggcctcaaaggtcaagagatgaacgccactggtggagatgaccccaggaatgcagctgggggctgtgaacgggaacctcagagcctgaccagagtggacctcagctcctctgtcctcccgggagactccgtgggactggcagtcattctgcacactgacagccgcaaggattccccccctgcgggttcaccagctggacggtccatctacaactccgcctatgtgtactgcaagggaccctgccagagggtgcagccgggaaagctcagagtgcagtgcagcacttgcagacaagccaccctgaccctgacccagggcccatcctgctgggatgatgtcctgatccccaaccggatgtcaggggaatgccaaagccctcactgccctggaacctcggccgagttcttcttcaaatgtggagcccaccccacctcggacaaggaaacctcggtggcccttcacctcattgccaccaactcccgcaacatcacctgtatcacttgcactgatgttcgctctccggtgctggtgttccagtgcaactcccgacacgtgatctgcctggactgcttccacctgtactgtgtgaccagactgaatgacaggcagtttgtccacgacccccaactgggctactccttgccttgtgtggctggctgccccaactccctgatcaaggagttgcaccacttccggatcctgggagaggaacagtacaacagataccagcagtacggggcagaggaatgtgtcctccaaatggggggagtgctgtgcccccggcctggttgtggagctggcctcctgccggaacctgaccagcggaaggtcacttgcgagggtggaaacggcctgggctgtggcttcgccttctgtcgggagtgcaaggaggcctaccacgaaggagaatgctccgcggtgtttgaagcctcagggaccaccacacaagcctacagagtggatgagagggcagcggagcaggcccgcgccgaagcggcctccaaggagactatcaagaaaaccaccaagccatgccctaggtgccatgtgcctgtggaaaagaatggaggctgcatgcacatgaagtgcccccagccacagtgccgccttgaatggtgctggaactgtggctgcgagtggaacagagtgtgtatgggggaccactggtttgatgtgtgataatggattcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggacgctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttcaagcaattctcctgcctcagcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattatgttgaaaacatagggtctggggaaaaagggatttaaaataagaagaaaaagaagacttgggacttaaaaagtcttttagaggccagctcaccaacattcagaacacccagtctgtgttgcacaatatgttacttaggtataaatcaaggattcatgtaattttgtcattccttgcgtgatattttaaaaaacattctgtgtaaggtatttataaagctctcttctaaaaatacaaaaatttgtggggccttgtagtcccagctacttgggaggctgaggcaggaggatttcttgaactgggaggcagagcttgcagtgagcccactgcatcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggcc

SEQ ID NO: 85 - DNA, 4431 bp, Artificial Sequence
  Made in Lab - Expression cassette
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcaggggaattcaaagcaagccgccaccatggcggtgcgacaggcgctgggccgcggcctgcagctgggtcgagcgctgctgctgcgcttcacgggcaagcccggccgggcctacggcttggggcggccgggcccggcggcgggctgtgtccgcggggagcgtccaggctgggccgcaggaccgggcgcggagcctcgcagggtcgggctcgggctccctaaccgtctccgcttcttccgccagtcggtggccgggctggcggcgcggttgcagcggcagttcgtggtgcgggcctggggctgcgcgggcccttgcggccgggcagtctttctggccttcgggctagggctgggcctcaatctacaacagcgcctatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgtgccgaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtagtaatggattcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggacgctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttcaagcaattctcctgcctcagcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattatgttgaaaacatagggtctggggaaaaagggatttaaaataagaagaaaaagaagacttgggacttaaaaagtcttttagaggccagctcaccaacattcagaacacccagtctgtgttgcacaatatgttacttaggtataaatcaaggattcatgtaattttgtcattccttgcgtgatattttaaaaaacattctgtgtaaggtatttataaagctctcttctaaaaatacaaaaatttgtggggccttgtagtcccagctacttgggaggctgaggcaggaggatttcttgaactgggaggcagagcttgcagtgagcccactgcatcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggcc

SEQ ID NO: 86 - DNA, 4431 bp, Artificial Sequence
  Made in Lab - Expression cassette
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcaggggaattcaaagcaagccgccaccatggctgtgcggcaggcactgggtcggggattgcaattgggaagagcattgcttctcagatttactggaaaaccaggaagagcatacgggttgggtcgccctggtcccgccgctgggtgcgtacgaggtgaacggccaggttgggctgcgggcccgggtgctgagccaagaagagtagggttgggattgcccaatcgccttaggttcttccggcagagcgtggctggtttggcagctagactccagaggcagtttgtagtgagggcctggggctgtgcaggcccatgcggacgagccgtatttttggctttcggacttgggttgggcctccatctacaactccgcctatgtgtactgcaagggaccctgccagagggtgcagccgggaaagctcagagtgcagtgcagcacttgcagacaagccaccctgaccctgacccagggcccatcctgctgggatgatgtcctgatccccaaccggatgtcaggggaatgccaaagccctcactgccctggaacctcggccgagttcttcttcaaatgtggagcccaccccacctcggacaaggaaacctcggtggcccttcacctcattgccaccaactcccgcaacatcacctgtatcacttgcactgatgttcgctctccggtgctggtgttccagtgcaactcccgacacgtgatctgcctggactgcttccacctgtactgtgtgaccagactgaatgacaggcagtttgtccacgacccccaactgggctactccttgccttgtgtggctggctgccccaactccctgatcaaggagttgcaccacttccggatcctgggagaggaacagtacaacagataccagcagtacggggcagaggaatgtgtcctccaaatggggggagtgctgtgcccccggcctggttgtggagctggcctcctgccggaacctgaccagcggaaggtcacttgcgagggtggaaacggcctgggctgtggcttcgccttctgtcgggagtgcaaggaggcctaccacgaaggagaatgctccgcggtgtttgaagcctcagggaccaccacacaagcctacagagtggatgagagggcagcggagcaggcccgcgccgaagcggcctccaaggagactatcaagaaaaccaccaagccatgccctaggtgccatgtgcctgtggaaaagaatggaggctgcatgcacatgaagtgcccccagccacagtgccgccttgaatggtgctggaactgtggctgcgagtggaacagagtgtgtatgggggaccactggtttgatgtgtgataatggattcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggacgctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttcaagcaattctcctgcctcagcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattatgttgaaaacatagggtctggggaaaaagggatttaaaataagaagaaaaagaagacttgggacttaaaaagtcttttagaggccagctcaccaacattcagaacacccagtctgtgttgcacaatatgttacttaggtataaatcaaggattcatgtaattttgtcattccttgcgtgatattttaaaaaacattctgtgtaaggtatttataaagctctcttctaaaaatacaaaaatttgtggggccttgtagtcccagctacttgggaggctgaggcaggaggatttcttgaactgggaggcagagcttgcagtgagcccactgcatcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggcc

SEQ ID NO: 87 - DNA, 4628 bp, Artificial Sequence
  Made in Lab - Expression cassette
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcaggggaattcaaagcaagccgccaccatggcggtgcgacaggcgctgggccgcggcctgcagctgggtcgagcgctgctgctgcgcttcacgggcaagcccggccgggcctacggcttggggcggccgggcccggcggcgggctgtgtccgcggggagcgtccaggctgggccgcaggaccgggcgcggagcctcgcagggtcgggctcgggctccctaaccgtctccgcttcttccgccagtcggtggccgggctggcggcgcggttgcagcggcagttcgtggtgcgggcctggggctgcgcgggcccttgcggccgggcagtctttctggccatggggctagggctgggcctcaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcgcctatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgtgccgaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtagtaatggattcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggacgctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttcaagcaattctcctgcctcagcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattatgttgaaaacatagggtctggggaaaaagggatttaaaataagaagaaaaagaagacttgggacttaaaaagtcttttagaggccagctcaccaacattcagaacacccagtctgtgttgcacaatatgttacttaggtataaatcaaggattcatgtaattttgtcattccttgcgtgatattttaaaaaacattctgtgtaaggtatttataaagctctcttctaaaaatacaaaaatttgtggggccttgtagtcccagctacttgggaggctgaggcaggaggatttcttgaactgggaggcagagcttgcagtgagcccactgcatcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggcc

SEQ ID NO: 88 - DNA, 4628 bp, Artificial Sequence
  Made in Lab - Expression cassette
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcaggggaattcaaagcaagccgccaccatggctgtgcggcaggcactgggtcggggattgcaattgggaagagcattgcttctcagatttactggaaaaccaggaagagcatacgggttgggtcgccctggtcccgccgctgggtgcgtacgaggtgaacggccaggttgggctgcgggcccgggtgctgagccaagaagagtagggttgggattgcccaatcgccttaggttcttccggcagagcgtggctggtttggcagctagactccagaggcagtttgtagtgagggcctggggctgtgcaggcccatgcggacgagccgtatttttggctatgggacttgggttgggcctcaaaggtcaagagatgaacgccactggtggagatgaccccaggaatgcagctgggggctgtgaacgggaacctcagagcctgaccagagtggacctcagctcctctgtcctcccgggagactccgtgggactggcagtcattctgcacactgacagccgcaaggattccccccctgcgggttcaccagctggacggtccatctacaactccgcctatgtgtactgcaagggaccctgccagagggtgcagccgggaaagctcagagtgcagtgcagcacttgcagacaagccaccctgaccctgacccagggcccatcctgctgggatgatgtcctgatccccaaccggatgtcaggggaatgccaaagccctcactgccctggaacctcggccgagttcttcttcaaatgtggagcccaccccacctcggacaaggaaacctcggtggcccttcacctcattgccaccaactcccgcaacatcacctgtatcacttgcactgatgttcgctctccggtgctggtgttccagtgcaactcccgacacgtgatctgcctggactgcttccacctgtactgtgtgaccagactgaatgacaggcagtttgtccacgacccccaactgggctactccttgccttgtgtggctggctgccccaactccctgatcaaggagttgcaccacttccggatcctgggagaggaacagtacaacagataccagcagtacggggcagaggaatgtgtcctccaaatggggggagtgctgtgcccccggcctggttgtggagctggcctcctgccggaacctgaccagcggaaggtcacttgcgagggtggaaacggcctgggctgtggcttcgccttctgtcgggagtgcaaggaggcctaccacgaaggagaatgctccgcggtgtttgaagcctcagggaccaccacacaagcctacagagtggatgagagggcagcggagcaggcccgcgccgaagcggcctccaaggagactatcaagaaaaccaccaagccatgccctaggtgccatgtgcctgtggaaaagaatggaggctgcatgcacatgaagtgcccccagccacagtgccgccttgaatggtgctggaactgtggctgcgagtggaacagagtgtgtatgggggaccactggtttgatgtgtgataatggattcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggacgctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttcaagcaattctcctgcctcagcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattatgttgaaaacatagggtctggggaaaaagggatttaaaataagaagaaaaagaagacttgggacttaaaaagtcttttagaggccagctcaccaacattcagaacacccagtctgtgttgcacaatatgttacttaggtataaatcaaggattcatgtaattttgtcattccttgcgtgatattttaaaaaacattctgtgtaaggtatttataaagctctcttctaaaaatacaaaaatttgtggggccttgtagtcccagctacttgggaggctgaggcaggaggatttcttgaactgggaggcagagcttgcagtgagcccactgcatcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggcc

SEQ ID NO: 89 - protein, 500 aa, Artificial Sequence
  Made in Lab - fusion protein
MAVRQALGRGLQLGRALLLRFTGKPGRAYGLGRPGPAAGCVRGERPGWAAGPGAEPRRVGLGLPNRLRFFRQSVAGLAARLQRQFVVRAWGCAGPCGRAVFLAMGLGLGLKGQEMNATGGDDPRNAAGGCEREPQSLTRVDLSSSVLPGDSVGLAVILHTDSRKDSPPAGSPAGRSIYNSAYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARAEAASKETIKKTTKPCPRCHVPVEKNGGCMHMKCPQPQCRLEWCWNCGCEWNRVCMGDHWFDV

SEQ ID NO: 90 - protein, 465 aa, Artificial Sequence
  Made in Lab - mutated Parkin protein
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGFMHMKCPQPQCRLEWCWNCGCEWNRVCMGDHWFDV

SEQ ID NO: 91 - DNA, 4494 bp, Artificial Sequence
  Made in Lab - Expression cassette
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacatacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagggggaattcaaagcaagccgccaccatgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgttgggaagcagcctccaaagaaaccatcaagaaaaccaccaagccctgtccccgctgccatgtaccagtggaaaaaaatggaggcttcatgcacatgaagtgtccgcagccccagtgcaggctcgagtggtgctggaactgtggctgcgagtggaaccgcgtctgcatgggggaccactggttcgacgtgggcggcggcggcagcggcggcggcggcagcatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgataatggagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 92 - DNA, 5457 bp, Artificial Sequence
  Made in Lab - Expression cassette
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcaggggaattcaaagcaagccgccaccatggcggtgcgacaggcgctgggccgcggcctgcagctgggtcgagcgctgctgctgcgcttcacgggcaagcccggccgggcctacggcttggggcggccgggcccggcggcgggctgtgtccgcggggagcgtccaggctgggccgcaggaccgggcgcggagcctcgcagggtcgggctcgggctccctaaccgtctccgcttcttccgccagtcggtggccgggctggcggcgcggttgcagcggcagttcgtggtgcgggcctggggctgcgcgggcccttgcggccgggcagtctttctggccttcgggctagggctgggcctcaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcgcctatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcaggggaattcaaagcaagccgccaccatgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaaggatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgtgccgaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactgggccgacgtgtgataatggatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggcc

SEQ ID NO: 93 - protein, 465 aa, Artificial Sequence; identical to SEQ ID NO: 13
  Made in Lab - mutated Parkin protein

SEQ ID NO: 94 - DNA, 3180 bp, Artificial Sequence
  Made in Lab - Expression cassette
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcaggggaattcaaagcaagccgccaccatgtcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgtgccgaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactgggccgacgtgtgataatggatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggcc

SEQ ID NO: 95 - protein, 325 aa, Artificial Sequence
  Made in Lab - mutated Parkin protein
SIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARAEAASKETIKKTTKPCPRCHVPVEKNGGCMHMKCPQPQCRLEWCWNCGCEWNRVCMGDHWADV

SEQ ID NO: 96 - DNA, 3702 bp, Artificial Sequence
  Made in Lab - Expression cassette
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcaggggaattcaaagcaagccgccaccatggcggtgcgacaggcgctgggccgcggcctgcagctgggtcgagcgctgctgctgcgcttcacgggcaagcccggccgggcctacggcttggggcggccgggcccggcggcgggctgtgtccgcggggagcgtccaggctgggccgcaggaccgggcgcggagcctcgcagggtcgggctcgggctccctaaccgtctccgcttcttccgccagtcggtggccgggctggcggcgcggttgcagcggcagttcgtggtgcgggcctggggctgcgcgggcccttgcggccgggcagtctttctggccttcgggctagggctgggcctcaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgtgccgaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactgggccgacgtgtgataatggatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggcc

SEQ ID NO: 97 - protein, 500 aa, Artificial Sequence
  Made in Lab - mutated Parkin protein
MAVRQALGRGLQLGRALLLRFTGKPGRAYGLGRPGPAAGCVRGERPGWAAGPGAEPRRVGLGLPNRLRFFRQSVAGLAARLQRQFVVRAWGCAGPCGRAVFLAFGLGLGLKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWADV

SEQ ID NO: 98 - DNA, 3702 bp, Artificial Sequence
  Made in Lab - Expression cassette
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcaggggaattcaaagcaagccgccaccatggcggtgcgacaggcgctgggccgcggcctgcagctgggtcgagcgctgctgctgcgcttcacgggcaagcccggccgggcctacggcttggggcggccgggcccggcggcgggctgtgtccgcggggagcgtccaggctgggccgcaggaccgggcgcggagcctcgcagggtcgggctcgggctccctaaccgtctccgcttcttccgccagtcggtggccgggctggcggcgcggttgcagcggcagttcgtggtgcgggcctggggctgcgcgggcccttgcggccgggcagtctttctggccatggggctagggctgggcctcaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgtgccgaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactgggccgacgtgtgataatggatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggcc

SEQ ID NO: 99 - protein, 500 aa, Artificial Sequence
  Made in Lab - mutated Parkin protein
MAVRQALGRGLQLGRALLLRFTGKPGRAYGLGRPGPAAGCVRGERPGWAAGPGAEPRRVGLGLPNRLRFFRQSVAGLAARLQRQFVVRAWGCAGPCGRAVFLAMGLGLGLKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWADV

(19) United States
(12) Patent Application Publication
(10) Pub. No.: US 2023/0174994 A1
(43) Pub. Date: Jun. 8, 2023

(54) ENGINEERED PARKIN AND USES THEREOF

(71) Applicant: Spacecraft Seven, LLC, Cranbury, NJ (US)

(72) Inventors: Chester Bittencort SACRAMENTO, Cranbury, NJ (US); Christopher Dean HERZOG, Cranbury, NJ (US); Raj PRABHAKAR, Cranbury, NJ (US)

(73) Assignee: Spacecraft Seven, LLC, Cranbury, NJ (US)

(21) Appl. No.: 17/926,024

(22) PCT Filed: May 20, 2021
(86) PCT No.: PCT/US2021/033491

§ 371 Date: Nov. 17, 2022

Related U.S. Application Data
(60) Provisional application No. 63/027,868, filed on May 20, 2020.
(60) Provisional application No. 63/027,866, filed on May 20, 2020.

(51) Int. Cl. C12N 15/62; C12N 15/86; C07K 14/005

(52) U.S. Cl. CPC C12N 15/62; C07K 14/005; C12N 15/86; C07K 2319/03; C07K 2319/07; C12N 2750/14143

(57) ABSTRACT

Parkin protein variants having activating mutations and/or fused to a mitochondrial targeting sequence are provided. The engineered Parkin may be a fusion protein including a mitochondrial targeting sequence (MTS); a transmembrane domain; and a Parkin protein or functional variant or fragment thereof, such as a Parkin having an N-terminal deletion. The MTS may be the MTS of PINK1 or a functional variant thereof. Alternatively or in addition, the engineered Parkin may have one or more activating mutations, such as single amino-acid substitutions. The engineered Parkin may be delivered in a vector, such as an adeno-associated virus (AAV) vector, and may be used to treat a disease or disorder, such as Parkinson’s disease or any of various neurodegenerative diseases.

### CROSS-REFERENCE TO RELATED APPLICATIONS

This application claims priority to U.S. Provisional Application No. 63/027,866, filed May 20, 2020, and U.S. Provisional Application No. 63/027,868, filed May 20, 2020, the contents of which are incorporated by reference herein in their entireties.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is ROPA_016_01WO_ST25.txt. The text file is about 265 KB, created on May 20, 2021, and is being submitted electronically via EFS-Web.

### FIELD OF THE INVENTION

The invention relates generally to gene therapy for disorders associated with mitochondrial dysfunction, e.g., central nervous system (CNS) disorders such as Parkinson’s disease. In particular, the disclosure provides engineered Parkin protein variants having activating mutations and/or fused to a mitochondrial targeting sequence.

### BACKGROUND

PARK2, which encodes the protein Parkin, is one of several genes implicated in Parkinson’s disease. Others include PARK1 (encoding the protein α-synuclein), PARK6 (encoding the protein PINK1), PARK7 (encoding the protein DJ-1), and PARK8 (encoding the protein LRRK2, also known as dardarin). Creed et al. (2018) Mov Disord. 33:717-729; Blesa et al. (2014) Front. Neuroanat. 8:1-12; Alcalay et al. (2010) Arch Neurol. 67:1116-1122).

PINK1 and Parkin together protect mitochondria from oxidative stress. PINK1 is translocated into mitochondria via an N-terminal mitochondrial signaling sequence (MTS). Absent mitochondrial stress, PINK1 is proteolytically cleaved within the healthy mitochondria by mitochondrial processing peptidase (MMP) and protease presenilin-associated rhomboid-like protein (PARL). Upon mitochondrial damage, PINK1 fails to fully translocate and instead accumulates at the mitochondrial surface with its transmembrane domain (TMD) embedded in the membrane of the damaged mitochondrial and protected from proteolysis from MMP and PARL.

Uncleaved PINK1 then serves to activate Parkin via sequential enzymatic steps. Various point mutations to Parkin have been shown to artificially activate Parkin without PINK1 activity, or to shift the equilibrium towards activation when PINK1 is active.

There is a long-felt and unmet need for gene therapy-based treatments for Parkinson’s disease and other disorders associated with mitochondrial dysfunction. The gene therapies provided here address this need.

### SUMMARY

In an aspect, the disclosure provides a recombinant adeno-associated virus (rAAV) virion, comprising a capsid and a vector genome, wherein the vector genome comprises a polynucleotide sequence encoding an activated Parkin protein operatively linked to a promoter.

In another aspect, the disclosure provides a method of increasing Parkin activity, e.g., in a cell, comprising contacting a cell with an rAAV virion of the disclosure.

In another aspect, the disclosure provides a method of increasing Parkin activity, e.g., in a cell, comprising administering to a subject an rAAV virion of the disclosure.

In another aspect, the disclosure provides a method of promoting survival of a neuron, comprising contacting the neuron with an rAAV virion of the disclosure.

In another aspect, the disclosure provides a method of promoting survival of a neuron, comprising administering to a subject an rAAV virion of the disclosure.

In another aspect, the disclosure provides a method of treating a disease or disorder, comprising administering to a subject an rAAV virion of the disclosure.

In another aspect, the disclosure provides a polynucleotide, comprising a polynucleotide sequence encoding a fusion protein comprising a mitochondrial targeting sequence (MTS); a transmembrane domain (TMD); and a Parkin protein or functional variant or fragment thereof.

In another aspect, the disclosure provides a vector comprising a polynucleotide of the disclosure.

In another aspect, the disclosure provides a method of increasing Parkin activity, e.g., in a cell, comprising administering to a subject a polynucleotide or vector of the disclosure.

In another aspect, the disclosure provides a method of promoting survival of a neuron, comprising contacting the neuron with a polynucleotide or vector of the disclosure.

In another aspect, the disclosure provides a method of promoting survival of a neuron, comprising administering to a subject a polynucleotide or vector of the disclosure.

In another aspect, the disclosure provides a method of treating a disease or disorder, comprising administering to a subject a polynucleotide or vector of the disclosure.

In further aspects, the disclosure provides cells, proteins, pharmaceutical compositions, and kits comprising or encoded by a polynucleotide or vector of the disclosure.

In further aspects, the disclosure provides pharmaceutical compositions and kits comprising an rAAV virion of the disclosure.

In various embodiments, the disclosure provides a polynucleotide that comprises a polynucleotide sequence encoding a fusion protein comprising a mitochondrial targeting sequence (MTS); a transmembrane domain (TMD); and a Parkin protein or functional variant or fragment thereof.

In some embodiments of the polynucleotide, the MTS is the MTS of PINK1 or a functional variant thereof.

In some embodiments of the polynucleotide, the MTS comprises a mitochondrial processing peptidase (MPP) cleavage site.

In some embodiments of the polynucleotide, the MTS comprises a polypeptide sequence at least 95% identical to resides 1-34 of human PINK1:

1 MAVRQALGRG LQLGRALLLR FTGKPGRAYG LGRP (SEQ ID NO:66).

In some embodiments of the polynucleotide, the MTS comprises a polypeptide sequence at least 95% identical to residues 1-94 of human PINK1:

1 MAVRQALGRG LQLGRALLLR FTGKPGRAYG LGRPGPAAGC
41 VRGERPGWAA GPGAEPRRVG LGLPNRLRFF RQSVAGLAAR
81 LQRQFVVRAW GCAG (SEQ ID NO: 65).

In some embodiments of the polynucleotide, the MTS comprises a polypeptide sequence identical to residues 1-94 of human PINK1:

1 MAVRQALGRG LQLGRALLLR FTGKPGRAYG LGRPGPAAGC
41 VRGERPGWAA GPGAEPRRVG LGLPNRLRFF RQSVAGLAAR
81 LQRQFVVRAW GCAG (SEQ ID NO: 65).

In some embodiments of the polynucleotide, the TMD is the TMD of PINK1 or a functional variant thereof.

In some embodiments of the polynucleotide, the TMD comprises a PARL cleavage site.

In some embodiments of the polynucleotide, the TMD comprises a polypeptide sequence at least 95% identical to residues 95-110 of human PINK1:

81 PCGRAV FLAFGLGLGL (SEQ ID NO: 67).

In some embodiments of the polynucleotide, the TMD comprises a polypeptide sequence identical to residues 95-110 of human PINK1:

81 PCGRAV FLAFGLGLGL (SEQ ID NO: 67).

In some embodiments of the polynucleotide, the TMD comprises a polypeptide sequence identical to residues 95-110 of human PINK1:

81 PCGRAV FLAMGLGLGL (SEQ ID NO: 68).

In some embodiments of the polynucleotide, the fusion protein comprises an MTS-TMD fragment of PINK1 or a functional variant thereof.

In some embodiments of the polynucleotide, the MTS-TMD fragment comprises a polypeptide sequence at least 95% identical to residues 1-110 of human PINK1:

1 MAVRQALGRG LQLGRALLLR FTGKPGRAYG LGRPGPAAGC
41 VRGERPGWAA GPGAEPRRVG LGLPNRLRFF RQSVAGLAAR
81 LQRQFVVRAW GCAGPCGRAV FLAFGLGLGL (SEQ ID NO: 70).

In some embodiments of the polynucleotide, the MTS-TMD fragment comprises a polypeptide sequence identical to residues 1-110 of human PINK1:

1 MAVRQALGRG LQLGRALLLR FTGKPGRAYG LGRPGPAAGC
41 VRGERPGWAA GPGAEPRRVG LGLPNRLRFF RQSVAGLAAR
81 LQRQFVVRAW GCAGPCGRAV FLAFGLGLGL (SEQ ID NO: 70).

In some embodiments of the polynucleotide, the functional variant or fragment thereof is a ΔParkin protein comprising a deletion of the N-terminal ubiquitin-like (Ubl) domain and optionally a deletion of the Ub1-RING0 interdomain linker sequence.

In some embodiments of the polynucleotide, the ΔParkin protein comprises a polypeptide sequence at least 95% identical to residues 141-465 of human Parkin F146A+W403A:

121 SIYNSAYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARAEAASKET IKKTTKPCPR CHVPVEKNGG CMHMKCPQPQ
441 CRLEWCWNCG CEWNRVCMGD HWFDV (SEQ ID NO: 73).

In some embodiments of the polynucleotide, the ΔParkin protein comprises a polypeptide sequence identical to residues 141-465 of human Parkin F146A+W403A:

121 SIYNSAYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARAEAASKET IKKTTKPCPR CHVPVEKNGG CMHMKCPQPQ
441 CRLEWCWNCG CEWNRVCMGD HWFDV (SEQ ID NO: 73).

In some embodiments of the polynucleotide, the ΔParkin protein comprises a polypeptide sequence at least 95% identical to residues 76-465 of human Parkin F146A+W403A:

41 KGQEM
81 NATGGDDPRN AAGGCEREPQ SLTRVDLSSS VLPGDSVGLA
121 VILHTDSRKD SPPAGSPAGR SIYNSAYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARAEAASKET IKKTTKPCPR CHVPVEKNGG CMHMKCPQPQ
441 CRLEWCWNCG CEWNRVCMGD HWFDV (SEQ ID NO: 74).

In some embodiments of the polynucleotide, the ΔParkin protein comprises a polypeptide sequence identical to residues 76-465 of human Parkin F146A+W403A:

41 KGQEM
81 NATGGDDPRN AAGGCEREPQ SLTRVDLSSS VLPGDSVGLA
121 VILHTDSRKD SPPAGSPAGR SIYNSAYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARAEAASKET IKKTTKPCPR CHVPVEKNGG CMHMKCPQPQ
441 CRLEWCWNCG CEWNRVCMGD HWFDV (SEQ ID NO:74).

In some embodiments of the polynucleotide, the fusion protein comprises an F146A substitution relative to a reference human Parkin protein sequence of SEQ ID NO: 1.

In some embodiments of the polynucleotide, the fusion protein comprises a W403A substitution relative to a reference human Parkin protein sequence of SEQ ID NO: 1.

In some embodiments of the polynucleotide, the fusion protein comprises an F463A substitution relative to a reference human Parkin protein sequence of SEQ ID NO: 1.

In some embodiments of the polynucleotide, the fusion protein comprises a C457S substitution relative to a reference human Parkin protein sequence of SEQ ID NO: 1.

In some embodiments of the polynucleotide, the fusion protein comprises both an F146A substitution and a W403A substitution relative to a reference human Parkin protein sequence of SEQ ID NO: 1.

In some embodiments of the polynucleotide, the fusion protein comprises a F104M substitution relative to a reference human PINK1 protein sequence of SEQ ID NO: 64.

In some embodiments of the polynucleotide, the fusion protein comprises both an F146A substitution and a W403A substitution relative to a reference human Parkin protein sequence of SEQ ID NO: 1, and wherein the fusion protein comprises a F104M substitution relative to a reference human PINK1 protein sequence of SEQ ID NO: 64.

In some embodiments of the polynucleotide, the fusion protein comprises a polypeptide sequence at least 95% identical to the sequence of SEQ ID NO: 97 or 98 and comprises two or more amino acid substitutions selected from F104M, W403A, and F463A. The F104M is relative to a reference human PINK1 protein sequence of SEQ ID NO: 64; W403A is relative to a reference human Parkin protein sequence of SEQ ID NO: 1; and F463A is relative to a reference human Parkin protein sequence of SEQ ID NO: 1.

In some embodiments of the polynucleotide, the fusion protein comprises a polypeptide sequence identical to the sequence any one of SEQ ID NO: 97 or 98 and comprises two or more amino acid substitutions selected from F104M, W403A, and F463A. The F104M is relative to a reference human PINK1 protein sequence of SEQ ID NO: 64; W403A is relative to a reference human Parkin protein sequence of SEQ ID NO: 1; and F463A is relative to a reference human Parkin protein sequence of SEQ ID NO: 1.

In various embodiments, the disclosure provides a vector that comprises a polynucleotide of the embodiments.

In some embodiments of the vector, the vector is an adeno-associated virus (AAV) vector.

In some embodiments of the AAV vector, the vector comprises an AAV9 capsid or functional variant thereof. The AAV9 capsid may share at least 98%, 99%, or 100% identity to a reference AAV9 capsid.

In various embodiments, the disclosure provides a method of increasing Parkin activity in a cell, the method comprising contacting the cell with a polynucleotide or a vector of any of the embodiments.

In various embodiments, the disclosure provides a method of increasing Parkin activity in a subject, comprising administering to the subject a polynucleotide or a vector of any of the embodiments.

In some embodiments of the method, the cell or subject is deficient in Parkin activity and/or comprises a loss-of-function mutation in Parkin.

In some embodiments of the method, Parkin activity comprises one or more of colocalization of Parkin with TOMM2 in response to neurotoxin treatment, ubiquitination of mitochondrial proteins in response to neurotoxin treatment, and increased in Parkin levels in the mitochondrial fraction in response to neurotoxin treatment.

In various embodiments, the disclosure provides a method of promoting survival of a neuron, comprising contacting the neuron with a polynucleotide or a vector of any of the embodiments.

In various embodiments, the disclosure provides a method of promoting survival of a neuron in a subject, comprising administering to the subject a polynucleotide or a vector of any of the embodiments.

In some embodiments of the method, the neuron is a dopaminergic neuron.

In various embodiments, the disclosure provides a method of treating a disease or disorder in a subject in need thereof, comprising administering to the subject a polynucleotide or vector of any embodiment.

In some embodiments of the method, the subject suffers from a genetic deficiency in Parkin expression or function.

In some embodiments of the method, the subject suffers from a genetic deficiency in PINK1 expression or function.

In some embodiments of the method, the disease or disorder is Parkinson’s disease.

In some embodiments of the method, the Parkinson’s disease is early onset Parkinson’s disease (EOPD).

In some embodiments of the method, the method alleviates one or more symptoms of Parkinson’s disease.

In some embodiments of the method, the method reduces motor complications associated with neurodegeneration; reduces the need for antiparkinsonian pharmacotherapy, optionally L-DOPA and/or dopaminergic agonists; restores the function of degenerating neurons; and/or protects neurons from degeneration.

In some embodiments of the method, the method enhances nigrostriatal function, optionally assessed by [18F]fluoro-L-dopa positron emission tomography (PET) or DaT-SPECT imaging.

In some embodiments of the method, the method improves one or both of the UPDRS or MDS-UPDRS of the subject.

In various embodiments, the disclosure provides a cell comprising a polynucleotide of any embodiment.

In various embodiments, the disclosure provides a protein encoded by a polynucleotide of any embodiment.

In various embodiments, the disclosure provides a pharmaceutical composition comprising a vector of any embodiment and one or more pharmaceutically acceptable carriers, diluents, or excipients.

In various embodiments, the disclosure provides a kit comprising a vector of any embodiment and instructions for use.

In various embodiments, the disclosure provides a recombinant adeno-associated virus (rAAV) virion, comprising a capsid and a vector genome, wherein the vector genome comprises a polynucleotide sequence encoding an activated Parkin protein operatively linked to a promoter.

In some embodiments of the rAAV virion, the activated Parkin protein comprises one or more amino acid substitutions at positions Phe-146, Trp-403, Cys-457, Phe-463, and Asn-273 relative to a reference Parkin protein.

In some embodiments of the rAAV virion, the activated Parkin protein comprises two or more amino acid substitutions at positions Phe-146, Trp-403, Cys-457, Phe-463, and Asn-273 relative to a reference Parkin protein.

In some embodiments of the rAAV virion, the activated Parkin protein comprises amino acid substitutions at positions Phe-146, Trp-403, Cys-457, Phe-463, and Asn-273 relative to a reference Parkin protein.

In some embodiments of the rAAV virion, the activated Parkin protein comprises one or more amino acid substitutions selected from F146A, W403A, and/or N273K relative to a reference Parkin protein.

In some embodiments of the rAAV virion, the activated Parkin protein comprises amino acid substitutions F146A and W403A relative to a reference Parkin protein.

In some embodiments of the rAAV virion, the activated Parkin protein comprises amino acid substitutions F146A, N273K, and W403A relative to a reference Parkin protein.

In some embodiments of the rAAV virion, the activated Parkin protein comprises a polypeptide sequence at least 95% identical to human Parkin N273K+W403A+F463A (SEQ ID NO: 93).

In some embodiments of the rAAV virion, the activated Parkin protein comprises a polypeptide sequence identical to human Parkin N273K+W403A+F463A (SEQ ID NO: 93).

In some embodiments of the rAAV virion, the Parkin protein is a ΔParkin protein comprising a deletion of the ubiquitin-like (Ubl) domain.

In some embodiments of the rAAV virion, the ΔParkin protein comprises a polypeptide sequence at least 95% identical to residues 76-465 of human Parkin F146A+W403A:

41 KGQEM
81 NATGGDDPRN AAGGCEREPQ SLTRVDLSSS VLPGDSVGLA
121 VILHTDSRKD SPPAGSPAGR SIYNSAYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARAEAASKET IKKTTKPCPR CHVPVEKNGG CMHMKCPQPQ
441 CRLEWCWNCG CEWNRVCMGD HWFDV (SEQ ID NO: 18).

In some embodiments of the rAAV virion, the ΔParkin protein comprises a polypeptide sequence identical to residues 76-465 of human Parkin F146A+W403A:

41 KGQEM
81 NATGGDDPRN AAGGCEREPQ SLTRVDLSSS VLPGDSVGLA
121 VILHTDSRKD SPPAGSPAGR SIYNSAYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARAEAASKET IKKTTKPCPR CHVPVEKNGG CMHMKCPQPQ
441 CRLEWCWNCG CEWNRVCMGD HWFDV (SEQ ID NO: 18).

In some embodiments of the rAAV virion, the activated Parkin protein comprises amino acid substitutions at position Cys-431 relative to a reference Parkin protein.

In some embodiments of the rAAV virion, the activated Parkin protein comprises a C431F amino acid substitution relative to a reference Parkin protein.

In some embodiments of the rAAV virion, the promoter is a constitutive promoter

In some embodiments of the rAAV virion, the promoter is a CAG promoter.

In some embodiments of the rAAV virion, the promoter is a CMV promoter.

In some embodiments of the rAAV virion, the promoter is a neuron-specific promoter

In some embodiments of the rAAV virion, the promoter is a SYN promoter.

In some embodiments of the rAAV virion, the vector genome comprises a WPRE element.

In some embodiments of the rAAV virion, the vector genome comprises a hGH polyadenylation site.

In some embodiments of the rAAV virion, the capsid is an AAV9 capsid or functional variant thereof.

In some embodiments of the rAAV virion, the AAV9 capsid shares at least 98%, 99%, or 100% identity to a reference AAV9 capsid.

In various embodiments, the disclosure provides a method of increasing Parkin activity in a cell, comprising contacting the cell with an rAAV virion of any embodiment.

In various embodiments, the disclosure provides a method of increasing Parkin activity in a subject, comprising administering to the subject an effective amount of an rAAV virion of any embodiment.

In some embodiments of the method, the cell or subject is deficient in Parkin activity and/or comprises a loss-of-function mutation in Parkin.

In some embodiments of the method, Parkin activity comprises one or more of colocalization of Parkin with TOMM2 in response to neurotoxin treatment, ubiquitination of mitochondrial proteins in response to neurotoxin treatment, and increased in Parkin levels in the mitochondrial fraction in response to neurotoxin treatment.

In various embodiments, the disclosure provides a method of promoting survival of a neuron, comprising contacting the neuron with an rAAV virion of any embodiment.

In various embodiments, the disclosure provides a method of promoting survival of a neuron in a subject, comprising administering to the subject an effective amount of an rAAV virion of any embodiment.

In some embodiments of the method, the neuron is a dopaminergic neuron.

In various embodiments, the disclosure provides a method of treating a disease or disorder in a subject in need thereof, comprising administering to the subject an effective amount of an rAAV virion of any embodiment.

In some embodiments of the method, the subject suffers from a genetic deficiency in Parkin.

In some embodiments of the method, the subject suffers from a genetic deficiency in PINK1.

In some embodiments of the method, the subject suffers from a genetic deficiency in DJ-1.

In some embodiments of the method, the disease or disorder is Parkinson’s disease.

In some embodiments of the method, the Parkinson’s disease is early onset Parkinson’s disease (EOPD).

In some embodiments of the method, the method alleviates one or more symptoms of Parkinson’s disease.

In some embodiments of the method, the method reduces motor complications associated with neurodegeneration; reduces the need for antiparkinsonian pharmacotherapy, optionally L-DOPA and/or dopaminergic agonists; restores the function of degenerating neurons; and/or protects neurons from degeneration.

In some embodiments of the method, the method enhances nigrostriatal function, optionally assessed by [18F]fluoro-L-dopa positron emission tomography (PET) or DaT-SPECT imaging.

In some embodiments of the method, the method improves one or both of the UPDRS or MDS-UPDRS of the subject.

In various embodiments, the disclosure provides a pharmaceutical composition comprising an rAAV virion of any embodiment and one or more pharmaceutically acceptable carriers, diluents, or excipients.

In various embodiments, the disclosure provides a kit comprising an rAAV virion of any embodiment and instructions for use.

In various embodiments, the disclosure provides a polynucleotide, comprising a polynucleotide sequence encoding an activated Parkin protein.

In some embodiments of the polynucleotide, the activated Parkin protein comprises amino acid substitutions at position Cys-431 relative to a reference Parkin protein.

In some embodiments of the polynucleotide, the activated Parkin protein comprises a C431F amino acid substitution relative to a reference Parkin protein.

In some embodiments of the polynucleotide, the activated Parkin protein comprises one or more amino acid substitutions at positions Phe-146, Trp-403, Cys-457, Phe-463, and Asn-273 relative to a reference Parkin protein.

In some embodiments of the polynucleotide, the activated Parkin protein comprises two or more amino acid substitutions at positions Phe-146, Trp-403, Cys-457, Phe-463, and Asn-273 relative to a reference Parkin protein.

In some embodiments of the polynucleotide, the activated Parkin protein comprises amino acid substitutions at positions Phe-146, Trp-403, Cys-457, Phe-463, and Asn-273 relative to a reference Parkin protein.

In some embodiments of the polynucleotide, the activated Parkin protein comprises one or more amino acid substitutions selected from F146A, W403A, and/or N273K relative to a reference Parkin protein.

In some embodiments of the polynucleotide, the activated Parkin protein comprises amino acid substitutions F146A and W403A relative to a reference Parkin protein.

In some embodiments of the polynucleotide, the activated Parkin protein comprises amino acid substitutions F146A, N273K, and W403A relative to a reference Parkin protein.

In some embodiments of the polynucleotide, the activated Parkin protein comprises a polypeptide sequence at least 95% identical to human Parkin N273K+W403A+F463A (SEQ ID NO: 93).

In some embodiments of the polynucleotide, the activated Parkin protein comprises a polypeptide sequence identical to human Parkin N273K+W403A+F463A (SEQ ID NO: 93).

In some embodiments of the polynucleotide, the Parkin protein is a ΔParkin protein comprising a deletion of the ubiquitin-like (Ubl) domain.

In some embodiments of the polynucleotide, the ΔParkin protein comprises a polypeptide sequence at least 95% identical to residues 76-465 of human Parkin F146A+W403A:

41 KGQEM
81 NATGGDDPRN AAGGCEREPQ SLTRVDLSSS VLPGDSVGLA
121 VILHTDSRKD SPPAGSPAGR SIYNSAYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARAEAASKET IKKTTKPCPR CHVPVEKNGG CMHMKCPQPQ
441 CRLEWCWNCG CEWNRVCMGD HWFDV (SEQ ID NO: 18).

In some embodiments of the polynucleotide, the ΔParkin protein comprises a polypeptide sequence identical to residues 76-465 of human Parkin F146A+W403A:

41 KGQEM
81 NATGGDDPRN AAGGCEREPQ SLTRVDLSSS VLPGDSVGLA
121 VILHTDSRKD SPPAGSPAGR SIYNSAYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARAEAASKET IKKTTKPCPR CHVPVEKNGG CMHMKCPQPQ
441 CRLEWCWNCG CEWNRVCMGD HWFDV (SEQ ID NO: 18).

In some embodiments of the polynucleotide, the polynucleotide comprises a promoter operably linked to the polynucleotide sequence encoding an activated Parkin protein.

In some embodiments of the polynucleotide, the promoter is a constitutive promoter.

In some embodiments of the polynucleotide, the promoter is a CAG promoter.

In some embodiments of the polynucleotide, the promoter is a CMV promoter.

In some embodiments of the polynucleotide, the promoter is a neuron-specific promoter

In some embodiments of the polynucleotide, the promoter is a SYN promoter.

In some embodiments of the polynucleotide, the vector genome comprises a WPRE element.

In some embodiments of the polynucleotide, the vector genome comprises a hGH polyadenylation site.

In various embodiments, the disclosure provides a vector, comprising a polynucleotide of any embodiment.

In some embodiments of the vector, the vector is an adeno-associated virus (AAV) vector.

In some embodiments of the AAV vector, the vector comprises an AAV9 capsid or functional variant thereof. The AAV9 capsid may shares at least 98%, 99%, or 100% identity to a reference AAV9 capsid.

In various embodiments, the disclosure provides a method of increasing Parkin activity in a cell, comprising contacting the cell with the polynucleotide or the vector of any one of the embodiments.

In various embodiments, the disclosure provides a method of increasing Parkin activity in a subject, comprising administering to the subject the polynucleotide or the vector of any one of the embodiments.

In some embodiments of the method, the cell or subject is deficient in Parkin activity and/or comprises a loss-of-function mutation in Parkin.

In some embodiments of the method, Parkin activity comprises one or more of colocalization of Parkin with TOMM2 in response to neurotoxin treatment, ubiquitination of mitochondrial proteins in response to neurotoxin treatment, and increased in Parkin levels in the mitochondrial fraction in response to neurotoxin treatment.

In various embodiments, the disclosure provides a method of promoting survival of a neuron, comprising contacting the neuron with a polynucleotide or vector of any embodiment.

In various embodiments, the disclosure provides a method of promoting survival of a neuron in a subject, comprising administering to the subject a polynucleotide or vector of any embodiment.

In some embodiments of the method, the neuron is a dopaminergic neuron.

In various embodiments, the disclosure provides a method of treating a disease or disorder in a subject in need thereof, comprising administering to the subject a polynucleotide or vector of any embodiment.

In some embodiments of the method, the subject suffers from a genetic deficiency in Parkin expression or function.

In some embodiments of the method, the subject suffers from a genetic deficiency in PINK1 expression or function.

In some embodiments of the method, the disease or disorder is Parkinson’s disease.

In some embodiments of the method, the Parkinson’s disease is early onset Parkinson’s disease (EOPD).

In some embodiments of the method, the method alleviates one or more symptoms of Parkinson’s disease.

In some embodiments of the method, the method reduces motor complications associated with neurodegeneration; reduces the need for antiparkinsonian pharmacotherapy, optionally L-DOPA and/or dopaminergic agonists; restores the function of degenerating neurons; and/or protects neurons from degeneration.

In some embodiments of the method, the method enhances nigrostriatal function, optionally assessed by [18F]fluoro-L-dopa positron emission tomography (PET) or DaT-SPECT imaging.

In some embodiments of the method, the method improves one or both of the UPDRS or MDS-UPDRS of the subject.

In various embodiments, the disclosure provides a cell comprising a polynucleotide of any embodiment.

In various embodiments, the disclosure provides a protein encoded by a polynucleotide of any embodiment.

In various embodiments, the disclosure provides a pharmaceutical composition comprising a vector of any embodiment and one or more pharmaceutically acceptable carriers, diluents, or excipients.

In various embodiments, the disclosure provides a kit comprising a vector of any embodiment and instructions for use.

Further aspects and embodiments of the invention will be apparent from the detailed description that follows.

### BRIEF DESCRIPTION OF FIGURES

FIG. 1 shows a domain diagram of Parkin with certain amino acid substitutions indicated by arrows.

FIG. 2 shows a vector diagram of a non-limiting example of a vector genome.

FIG. 3 shows a vector diagram of a non-limiting example of a vector genome. Amino-acid substitutions at F146A, N273K, and W403A are indicated by arrows.

FIG. 4 shows a vector diagram of a non-limiting example of a vector genome. Amino-acid substitutions at F146A and W403A are indicated by arrows.

FIG. 5 shows a vector diagram of a non-limiting example of a vector genome. Amino-acid substitutions at F146A and W403A are indicated by arrows.

FIG. 6 shows a vector diagram of a non-limiting example of a vector genome.

FIG. 7 shows a vector diagram of a non-limiting example of a vector genome. Amino-acid substitutions at F146A and W403A are indicated by arrows.

FIG. 8 shows a vector diagram of a non-limiting example of a vector genome. Amino-acid substitutions at F104M, F146A, and W403A are indicated by arrows. The F104M is relative to a reference human PINK1 protein sequence of SEQ ID NO: 64; W403A is relative to a reference human Parkin protein sequence of SEQ ID NO: 1; and F463A is relative to a reference human Parkin protein sequence of SEQ ID NO: 1.

FIG. 9 shows a vector diagram of a non-limiting example of a vector genome. An amino-acid substitution at C431F is indicated by an arrow.

FIGS. 10A-10D show testing of bioactivity of Parkin constructs in transfected N27A dopaminergic (DA) neurons. Luminescence Units (LU) measures neuronal proliferation and/or survival measured 3 days after treatment with control (FIG. 10A), 7.5 µM 6-hydroxydopamine (6-OHDA) (FIG. 10B), 15 µM 6-OHDA (FIG. 10C), or 30 µM 6-OHDA (FIG. 10D).

FIGS. 11A-11D show testing of bioactivity of Parkin constructs in transfected N27A dopaminergic (DA) neurons. Luminescence Units (LU) measures neuronal proliferation and/or survival measured 9 days after treatment with control (FIG. 11A), 7.5 µM 6-OHDA (FIG. 10B), 15 µM 6-OHDA (FIG. 10C), or 30 µM 6-OHDA (FIG. 10D).

FIG. 12 show testing of bioactivity of Parkin constructs in transfected human PARK2^{-/-} dopaminergic (DA) neurons

FIG. 13 shows a Western Blot of Parkin protein expression following transduction of primary neurons with AAV vectors encoding Parkin variants. CON GFP= Control green fluorescent protein, ACT= Activated Parkin, DEL= ΔParkin, SUP 1= Super Parkin, SUP2= Super Parkin V2, WT= Wild Type Parkin, C431F= C431F amino acid substitution.

FIG. 14 shows a vector diagram of a non-limiting example of a vector genome.

FIG. 15 shows a vector diagram of a non-limiting example of a vector genome.

FIG. 16 shows a vector diagram of a non-limiting example of a vector genome.

FIG. 17 shows a vector diagram of a non-limiting example of a vector genome.

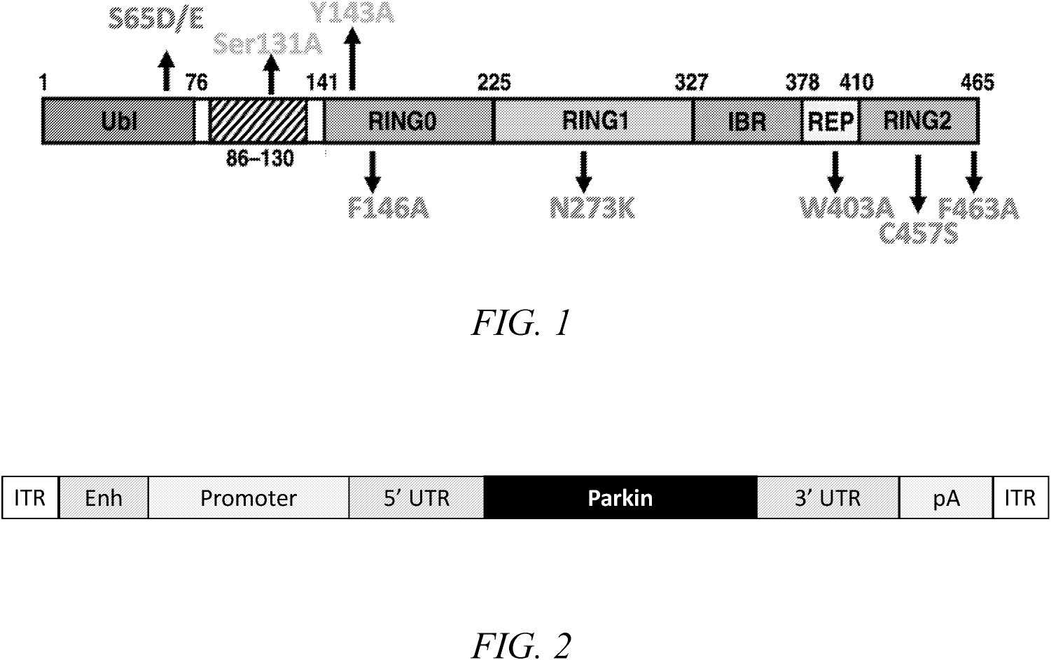

### DETAILED DESCRIPTION OF THE INVENTION

### Overview

Adeno-associated virus vectors, such as an AAV2 vector, have been used to deliver potentially therapeutic transgenes to the brain of subjects having Parkinson’s disease (PD), with limited success. For example, a recent double-blinded study of AAV2-neurturin delivery was well-tolerated but not superior to sham surgery. Olanow et al. Ann Neurol. 78:248-57 (2015). Parkin expression from AAV vectors has been shown to have neuroprotective effects on s substantia nigra dopamine neurons in preclinical models of neurodegeneration (Benskey et al., Neurotox, 2015; Paterna et al., Mol Ther, 2007; Yasuda et al., J Neuropath Exp Neurol, 2011; Klein et al. Neurosci Lett. 401:130-135 (2006). AAV-mediated gene delivery of Nurr1 and Foxa2 in a PD mouse model markedly protected midbrain DA (mDA) neurons and motor behaviors associated with nigrostriatal DA neurotransmission. Oh et al. EMBO Mol Med. 7:510-25 (2015).

The present invention relates generally to gene therapy for disorders associated with mitochondrial dysfunction, e.g., central nervous system (CNS) disorders, such as Parkinson’s disease. In particular, the disclosure provides recombinant adeno-associated virus (rAAV) virions for expression of an activated Parkin protein.

In one aspect, the disclosure provides recombinant adeno-associated virus (rAAV) virions comprising a capsid and a vector genome, where the vector genome comprises a polynucleotide sequence encoding an activated Parkin protein operatively linked to a promoter.

In other aspects, the disclosure provides methods of promoting survival of neurons comprising contacting the neurons with, or administering to a subject, the disclosed rAAV virions, optionally in an effective amount.

In another aspect, the disclosure provides methods of treating a disease or disorder comprising administering to the subject an effective amount of the disclosed rAAV virions.

Further, the disclosure provides polynucleotide sequence encoding a fusion protein where a portion of the Parkin protein is fused to a mitochondrial targeting sequence (MTS). Further provided are vectors, e.g. recombinant adeno-associated virus (rAAV) vectors, comprising the polynucleotides of the disclosure.

In one aspect, the disclosure provides a polynucleotide, comprising a polynucleotide sequence encoding a fusion protein comprising a mitochondrial targeting sequence (MTS); a transmembrane domain (TMD); and a Parkin protein or functional variant thereof.

In other aspects, the disclosure provides a vector comprising a polynucleotide of the disclosure.

In another aspect, the disclosure provides a method of increasing Parkin activity in a cell, comprising contacting the cell with a polynucleotide or a vector of the disclosure.

In another aspect, the disclosure provides a method of increasing Parkin activity in a subject, comprising administering to the subject a polynucleotide or a vector of the disclosure.

In another aspect, the disclosure provides a method of promoting survival of a neuron, comprising contacting the neuron with a polynucleotide or a vector of the disclosure.

In another aspect, the disclosure provides a method of promoting survival of a neuron in a subject, comprising administering to the subject a polynucleotide or a vector of the disclosure.

In another aspect, the disclosure provides a method of treating a disease or disorder in a subject in need thereof, comprising administering to the subject a polynucleotide or a vector of the disclosure.

Various other aspects and embodiments are disclosed in the detailed description that follows. The invention is limited solely by the appended claims.

### Definitions

The section headings are for organizational purposes only and are not to be construed as limiting the subject matter described to particular aspects or embodiments.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

As used herein, the terms “identity” and “identical” refer, with respect to a polypeptide or polynucleotide sequence, to the percentage of exact matching residues in an alignment of that “query” sequence to a “subject” sequence, such as an alignment generated by the BLAST algorithm. Identity is calculated, unless specified otherwise, across the full length of the subject sequence. Thus a query sequence “shares at least x% identity to” a subject sequence if, when the query sequence is aligned to the subject sequence, at least x% (rounded down) of the residues in the subject sequence are aligned as an exact match to a corresponding residue in the query sequence. Where the subject sequence has variable positions (e.g., residues denoted X), an alignment to any residue in the query sequence is counted as a match. Comparison of sequences to determine percent identity can be accomplished by a number of well-known methods, including for example by using mathematical algorithms, such as, for example, those in the BLAST suite of sequence analysis programs. Unless noted otherwise, the terms “identity” and “identical” to a reference sequence refers to sequence identity across the full length of the reference sequence after the two sequences are aligned using the Blast-p program (for proteins) or Blast-n program (for polynucleotides) of the National Center for Biotechnology Information (NCBI) online alignment tool, version 2.11.0 (released Oct. 19, 2020), available at blast.ncbi.nlm.nih.gov. See Altschul et al. J. Mol. Biol. 215:403-410 (1990).

As used herein, an “AAV vector” or “rAAV vector” refers to a recombinant vector comprising one or more polynucleotides of interest (or transgenes) that are flanked by AAV terminal repeat sequences (ITRs). Such AAV vectors can be replicated and packaged into infectious viral particles when present in a host cell that has been transfected with a plasmid encoding and expressing rep and cap gene products. Alternatively, AAV vectors can be packaged into infectious particles using a host cell that has been stably engineered to express rep and cap genes.

As used herein, an “AAV virion” or “AAV viral particle” or “AAV vector particle” refers to a viral particle composed of at least one AAV capsid protein and an encapsidated polynucleotide AAV vector. As used herein, if the particle comprises a heterologous polynucleotide (i.e., a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

As used herein, “promoter” refers to a polynucleotide sequence capable of promoting initiation of RNA transcription from a polynucleotide in a eukaryotic cell.

As used herein, “vector genome” refers to the polynucleotide sequence packaged by the vector (e.g., an rAAV virion), including flanking sequences (in AAV, inverted terminal repeats). The terms “expression cassette” and “polynucleotide cassette” refer to the portion of the vector genome between the flanking sequences. “Expression cassette” implies that the vector genome comprises at least one gene encoding a gene product operable linked to an element that drives expression (e.g., a promoter).

As used herein, the term “patient in need” or “subject in need” refers to a patient or subject at risk of, or suffering from, a disease, disorder or condition that is amenable to treatment or amelioration with a recombinant gene therapy vector or gene editing system disclosed herein. A patient or subject in need may, for instance, be a patient or subject diagnosed with a disorder associated with central nervous system degradation. A subject may have a mutation or a malfunction in a PARK2, PARK6, PARK7, LRRK2, or α-synuclein, gene or protein. “Subject” and “patient” are used interchangeably herein. The subject treated by the methods described herein may be an adult or a child. Subjects may range in age. The subject may be a person identified as at risk for a Parkinson’s Disease, e.g., an early-onset Parkinson’s Disease.

As used herein, “deficient in″-a such as a cell or subject “deficient in Parkin activity” -refers to either genetic deficiency due to partial complete loss of function in the PARK2 gene or to decrease in activity from other causes—e.g., expression of a protein (Parkin) at lower than normal levels, or decrease in expression of a factor that influences protein (Parkin) activity. For example, cells that express lower than normal levels of PINK1 may have decreased activity in Parkin, because PINK1 activates Parkin.

As used herein, “Parkin activity” refers to any enzymatic or cell signaling activity of Parkin.

As used herein, “activated Parkin” refers to variant of the Parkin protein having increased intrinsic activity in one or more biochemical or cellular assays compared to a reference Parkin protein (e.g., human Parkin protein).

As used herein, the term “variant” or “functional variant” refer, interchangeably, to a protein that has one or more amino-acid substitutions, insertions, or deletion compared to a parental protein that retains one or more desired activities of the parental protein.

As used herein, “genetic deficiency” refers to a partial or complete loss of function in a gene. For example, a subject that suffers from a genetic deficiency in Parkin expression of function has one or more mutations in the PARK2 gene that decreases expression or decreases the function of the Parkin protein in at least some cells (e.g., neurons) of the subject.

As used herein, “Parkinson’s disease” refers any of the forms of the disease known in the art by this name, as defined, e.g., in “The Differential Diagnosis of Parkinson’s Disease.” Parkinson’s Disease: Pathogenesis and Clinical Aspects, Chapter 6. Codon Publications (2018) or in Harrison’s Principles of Internal Medicine, 20^{th} ed.

As used herein, “treating” refers to inhibiting, reducing, or ameliorating one or more symptoms of a disease or disorder and/or preventing progression of a disease or disorder.

As used herein, the phrase “disease associated with mitochondrial dysfunction” refers to any disease or disorder whose development or progression related to dysfunction of mitochondrial that can be prevented or reversed by Parkin activity.

### Parkin Protein

The present disclosure contemplates compositions and methods of use related to various activated Parkin proteins. An activated Parkin protein is any Parkin protein having increased biochemical, cellular, or physiological activity compared to a reference Parkin protein (e.g., a wild-type Parkin protein, such as the Parkin protein normally encoded by the human PRKN2 gene, i.e., H1 in Table 1).

Further, the present disclosure contemplates compositions and methods of use related to various fusions of a portion of the Parkin protein to mitochondrial targeting sequence (MTS). The Parkin protein may optionally be a ΔParkin protein—that is, Parkin protein having a deletion of one or more domain relative to a reference Parkin protein (e.g., a wild-type Parkin protein, such as the Parkin protein normally encoded by the human PRKN2 gene, i.e., H1 in Table 1).

Alternative splicing generates various alternative isoforms of human Parkin, shown in Table 1 (see Scuderi et al. BioMed Res. Int’l Vol. 2014, Article 690796).

TABLE 1

Parkin isoforms

| New code identifier | Protein accession number | SEQ ID NO: | aa sequence | Predicted MW | pI |
|---|---|---|---|---|---|
| H20 | AGH62057.1 | | 530 aa | 58.127 | 6.41 |
| | BAA25751.1 | | | | |
| | BAF43729.1 | | | | |
| H1 | BAF85279.1 | | 465 aa | 51.65 | 6.71 |
| | NP_004553.2 | | | | |
| | ABN46990.1 | | | | |
| | ADB90270.1 | | 437 aa | 48.713 | 7.12 |
| H5 | NP 054642.2 | | 415 aa | 46.412 | 6.91 |
| H10 | ADB90271.1 | | | | |
| H14 | ADB91979.1 | | 387 aa | 43.485 | 7.43 |
| H4 | AAH22014.1 | | 387 aa | 42.407 | 8.15 |
| H8 | * | | 386 aa | 42.52 | 6.65 |
| H17 | * | | 386 aa | 42.52 | 6.65 |
| H21 | AGP25366.1 | | 358 aa | 39.592 | 7.08 |
| H6 | NP_054643.2 | | 316 aa | 35.63 | 6.45 |
| H11 | * | | 274 aa | 30.615 | 6.3 |
| H2 | AAM21457.1 | | 270 aa | 30.155 | 6.05 |
| H3 | AAM21459.1 | | 203 aa | 22.192 | 5.68 |
| H12 | * | | 172 aa | 19.201 | 6.09 |
| H9 | ADB90269.1 | | 143 aa | 15.521 | 5.54 |
| H13 | ADB91978.1 | | 143 aa | 15.521 | 5.54 |
| H7 | BAG57845.1 | | 139 aa | 15.407 | 6.41 |
| H18 | * | | 139 aa | 15.393 | 6.41 |
| H15 | ADB91980.1 | | 95 aa | 10.531 | 8.74 |
| H19 | AGH62056.1 | | 61 aa | 6.832 | 10.09 |
| H16 | ADB91981.1 | | 51 aa | 5.348 | 7.79 |

* The protein accession number is not present in database.

The polypeptide sequence of the canonical, human Parkin isoform (H1) is as follows:

1 MIVFVRFNSS HGFPVEVDSD TSIFQLKEW AKRQGVPADQ
41 LRVIFAGKEL RNDWTVQNCD LDQQSIVHIV QRPWRKGQEM
81 NATGGDDPRN AAGGCEREPQ SLTRVDLSSS VLPGDSVGLA
121 VILHTDSRKD SPPAGSPAGR SIYNSFYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARWEAASKET IKKTTKPCPR CHVPVEKNGG CMHMKCPQPQ
441 CRLEWCWNCG CEWNRVCMGD HWFDV

(SEQ ID NO: 1).

The reference Parkin protein may be SEQ ID NO: 1. The activated Parkin protein may also be another isoform of Parkin, e.g., having amino acid substitution(s) in an equivalent position in a multiple sequence alignment of Parkin protein isoform, prepared, e.g., with ClustalW or MUSCLE alignment algorithms.

Further isoforms of Parkin that may be used in the compositions and methods of the disclosure include the polypeptides of SEQ ID NOs: 2-8.

In some embodiments, the polynucleotide encoding the activated Parkin comprises a polynucleotide sequence at least 75%, 80%, 85%, 90%, 92%, 93%, 94%, 95%, 96%, 97%, 98%, 99%, or 100% identical to SEQ ID NO: 9.

The polynucleotide sequence encoding the activated Parkin may be codon-optimized. In some embodiments, the polynucleotide encoding the activated Parkin comprises a polynucleotide sequence at least 75%, 80%, 85%, 90%, 92%, 93%, 94%, 95%, 96%, 97%, 98%, 99%, or 100% identical to SEQ ID NO: 10.

In some embodiments, the activated Parkin comprises one or more amino acid substitutions selected from: mutation of residues in the predicted the Ubl (S65D or S65E), linker (S131A) RlNG0 (Y143A, F146A), RING1 (N273K), REP (W403A), or RING2 (C457S or F463A) domains, numbered relative to SEQ ID NO: 1. That is, the activated Parkin protein may comprises one or more of, two or more of, three or more, or four or more amino acid substitutions selected from the group consisting of S65D or S65E, S131A, Y143A, F146A, N273K, W403A, C457S, and F463A. Alternative conservative, or non-conservative mutations at any of these sites may be used, including without limitation one or more of, two or more of, three one or more, or four or more amino acid substitutions selected from the group consisting of S65X, S131X, Y143X, F146X, N273X, W403X, C457X, and F463X, where X represents any naturally or non-naturally occurring amino acid other than the amino acid present in the reference Parkin protein.

Particular mutations contemplated by the present disclosure include S65D, S65E, S65K, or S65R; S131A, S131L, or S131I; F146A, F146S, F146T, F146I, or F146L; N273K, N2773R, N273E, or N273Q; and F463A, F463S, F463T, F463I, or F463L. In some embodiments, the amino acid substitution disrupts an intra-molecular or inter-molecular interface. In some embodiments, the amino acid substitution disrupts an intra-molecular or inter-molecular interface, while maintaining one or more characteristics of the residue, such as charge, size, and/or hydrophobicity.

The activated Parkin may comprise one or more amino-acid substitutions, inserts, or deletions (collectively, mutations) that reduce the binding of one structural domain of Parkin to another, and thereby reduce autoinhibition. For example, the activated Parkin may comprise a mutation of in the Ubl that reduces binding to the RING1 domain or a mutation in the RING1 domain that reduces binding to the Ubl domain (e.g., N273K). The activated Parkin may comprise a mutation of in the REP domain that reduces binding to the RING1 domain (e.g., W403A) or a mutation in the RING1 domain that reduces binding to the REP domain. The activated Parkin may comprise a mutation of in the RlNG0 domain that reduces binding to the RING2 domain (e.g., F146A) or mutation in the RING2 domain that reduces binding to the RlNG0 domain (e.g., C457S and/or F463A).

Alternatively or in addition to the foregoing, the activated Parkin may comprise mutations that protect against degradation of Parkin mediated by kinase c-Abl (e.g., Y143A) or mediated by kinase p38MAPK (e.g., S131A).

Alternatively or in addition to the foregoing, the activated Parkin may comprise the amino acid substitution C431X, where X represents any naturally or non-naturally occurring amino acid other than the amino acid present in the reference Parkin protein. In some embodiments, the activated Parkin may comprise the amino acid substitution C431F.

Various further embodiments of the activated Parkin are provided in Table 2A or Table 2B.

TABLE 2A

Illustrative Combinations of Amino Acid Substitutions

| | | | |
|---|---|---|---|
| N273K + W403A + F146A | N273K + W403A + F146A + Y143A | N273K + W403A + F146A + Ser131A | N273K + W403A + F146A + Y143A+ Ser131A |
| N273K + W403A + C457S | N273K + W403A + C457S + Y143A | N273K + W403A + C457S + Ser131A | N273K + W403A + C457S + Y143A+ Ser131A |
| N273K + W403A + F463A | N273K + W403A + F463A + Y143A | N273K + W403A + F463A + Ser131A | N273K + W403A + F463A + Y143A + Ser131A |
| N273K + W403A + F146A + C457S | N273K + W403A + F146A + C457S + Y143A | N273K + W403A + F146A + C457S + Ser131A | N273K + W403A + F146A + C457S + Y143A + Ser131A |
| N273K + W403A + F146A + C457S + F463A | N273K + W403A + F146A + C457S + F463A + Y143A | N273K + W403A + F146A + C457S + F463A + Ser131A | N273K + W403A + F146A + C457S + F463A + Y143A+ Ser131A |
| W403A + F146A | W403A + F146A + Y143A | W403A + F146A + Ser131A | W403A + F146A + Y143A+ Ser131A |
| W403A + C457S | W403A + C457S + Y143A | W403A + C457S + Ser131A | W403A + C457S + Y143A+ Ser131A |
| W403A + F463A | W403A + F463A + Y143A | W403A + F463A + Ser131A | W403A + F463A + Y143A+ Ser131A |
| W403A + F146A + C457S | W403A + F146A + C457S + Y143A | W403A + F146A + C457S + Ser131A | W403A + F146A + C457S + Y143A+ Ser131A |
| W403A + F146A + C457S + F463A | W403A + F146A + C457S + F463A + Y143A | W403A + F146A + C457S + F463A + Ser131A | W403A + F146A + C457S + F463A + Y143A+ Ser131A |

TABLE 2B

Illustrative Combinations of Amino Acid Substitutions

| | | | |
|---|---|---|---|
| N273K + W403A + F146A + C431F | N273K + W403A + F146A + Y143A + C431F | N273K + W403A + F146A + Ser131A + C431F | N273K + W403A + F146A + Y143A+ Ser131A + C431F |
| N273K + W403A + C457S + C431F | N273K + W403A + C457S + Y143A + C431F | N273K + W403A + C457S + Ser131A + C431F | N273K + W403A + C457S + Y143A+ Ser131A + C431F |
| N273K + W403A + F463A + C431F | N273K + W403A + F463A + Y143A + C431F | N273K + W403A + F463A + Ser131A + C431F | N273K + W403A + F463A + Y143A+ Ser131A + C431F |
| N273K + W403A + F146A + C457S + C431F | N273K + W403A + F146A + C457S + Y143A + C431F | N273K + W403A + F146A + C457S + Ser131A + C431F | N273K + W403A + F146A + C457S + Y143A + Ser131A + C431F |
| N273K + W403A + F146A + C457S + F463A + C431F | N273K + W403A + F146A + C457S + F463A + Y143A + C431F | N273K + W403A + F146A + C457S + F463A + Ser131A + C431F | N273K + W403A + F146A + C457S + F463A + Y143A+ Ser131A + C431F |
| W403A + F146A + C431F | W403A + F146A + Y143A + C431F | W403A + F146A + Ser131A + C431F | W403A + F146A + Y143A+ Ser131A + C431F |
| W403A + C457S + C431F | W403A + C457S + Y143A + C431F | W403A + C457S + Ser131A + C431F | W403A + C457S + Y143A+ Ser131A + C431F |
| W403A + F463A + C431F | W403A + F463A + Y143A + C431F | W403A + F463A + Ser131A + C431F | W403A + F463A + Y143A+ Ser131A + C431F |
| W403A + F146A + C457S + C431F | W403A + F146A + C457S + Y143A + C431F | W403A + F146A + C457S + Ser131A + C431F | W403A + F146A + C457S + Y143A+ Ser131A + C431F |
| W403A + F146A + C457S + F463A + C431F | W403A + F146A + C457S + F463A + Y143A + C431F | W403A + F146A + C457S + F463A + Ser131A + C431F | W403A + F146A + C457S + F463A + Y143A+ Ser131A + C431F |

In some embodiments, the activated Parkin protein comprises one or more amino acid substitutions at position Cys-431 relative to a reference Parkin protein.

In some embodiments, the activated Parkin protein comprises one or more amino acid substitutions at positions Phe-146, Trp-403, Cys-457, Phe-463, and Asn-273 relative to a reference Parkin protein.

In some embodiments, the activated Parkin protein comprises two or more amino acid substitutions at positions Phe-146, Trp-403, Cys-457, Phe-463, and Asn-273 relative to a reference Parkin protein.

In some embodiments, the activated Parkin protein comprises amino acid substitutions at positions Phe-146, Trp-403, Cys-457, Phe-463, and Asn-273 relative to a reference Parkin protein.

In some embodiments, the activated Parkin protein comprises one or more amino acid substitutions selected from F146A, W403A, and/or N273K relative to a reference Parkin protein.

In some embodiments, the activated Parkin protein comprises amino acid substitutions F146A and W403A relative to a reference Parkin protein.

In some embodiments, the activated Parkin protein comprises amino acid substitutions F146A, N273K, and W403A relative to a reference Parkin protein.

In some embodiments, the activated Parkin protein comprises a polypeptide sequence at least 75%, 80%, 85%, 90%, 92%, 93%, 94%, 95%, 96%, 97%, 98% or 99% identical to an isoform of human Parkin listed in Table 1. In some embodiments, the activated Parkin protein comprises a polypeptide sequence at least 75%, 80%, 85%, 90%, 92%, 93%, 94%, 95%, 96%, 97%, 98% or 99% identical to the human Parkin of SEQ ID NO: 1.

In some embodiments, the activated Parkin protein comprises a polypeptide sequence at least 75%, 80%, 85%, 90%, 92%, 93%, 94%, 95%, 96%, 97%, 98%, 99% or 100% identical to human Parkin F146A+N273K+W403A:

1 MIVFVRFNSS HGFPVEVDSD TSIFQLKEVV AKRQGVPADQ
41 LRVIFAGKEL RNDWTVQNCD LDQQSIVHIV QRPWRKGQEM
81 NATGGDDPRN AAGGCEREPQ SLTRVDLSSS VLPGDSVGLA
121 VILHTDSRKD SPPAGSPAGR SIYNSA YVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLK DRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARA EAASKET IKKTTKPCPR CHVPVEKNGG CMHMKCPQPQ
441 CRLEWCWNCG CEWNRVCMGD HWFDV

(SEQ ID NO: 11).

In some embodiments, the activated Parkin protein consists of the polypeptide sequence of human Parkin F146A+N273K+W403A (SEQ ID NO: 11).

In some embodiments, the activated Parkin protein consists of a polypeptide sequence identical, across the full length of the polypeptide sequence, to a portion of human Parkin F146A+N273K+W403A (SEQ ID NO: 11), the polypeptide sequence having C-terminal and/or N-terminal truncations of 1, 2, 3, 4, 5, 6, 7, 8, 9, or 10 amino acids with respect to SEQ ID NO: 11.

In some embodiments, the activated Parkin protein comprises a polypeptide sequence at least 75%, 80%, 85%, 90%, 92%, 93%, 94%, 95%, 96%, 97%, 98%, 99% or 100% identical to human Parkin N273K+W403A+C457S:

1 MIVFVRFNSS HGFPVEVDSD TSIFQLKEVV AKRQGVPADQ
41 LRVIFAGKEL RNDWTVQNCD LDQQSIVHIV QRPWRKGQEM
81 NATGGDDPRN AAGGCEREPQ SLTRVDLSSS VLPGDSVGLA
121 VILHTDSRKD SPPAGSPAGR SIYNSFYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLK DRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARA EAASKET IKKTTKPCPR CHVPVEKNGG CMHMKCPQPQ
441 CRLEWCWNCG CEWNRVS MGD HWFDV

(SEQ ID NO: 12).

In some embodiments, the activated Parkin protein comprises a polypeptide sequence at least 75%, 80%, 85%, 90%, 92%, 93%, 94%, 95%, 96%, 97%, 98%, 99% or 100% identical to human Parkin N273K+W403A+F463A:

1 MIVFVRFNSS HGFPVEVDSD TSIFQLKEVV AKRQGVPADQ
41 LRVIFAGKEL RNDWTVQNCD LDQQSIVHIV QRPWRKGQEM
81 NATGGDDPRN AAGGCEREPQ SLTRVDLSSS VLPGDSVGLA
121 VILHTDSRKD SPPAGSPAGR SIYNSFYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLK DRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARA EAASKET IKKTTKPCPR CHVPVEKNGG CMHMKCPQPQ
441 CRLEWCWNCG CEWNRVCMGD HWA DV

(SEQ ID NO: 13).

In some embodiments, the activated Parkin protein comprises a polypeptide sequence at least 75%, 80%, 85%, 90%, 92%, 93%, 94%, 95%, 96%, 97%, 98%, 99% or 100% identical to human Parkin F146A+N273K+W403A+C457S:

1 MIVFVRFNSS HGFPVEVDSD TSIFQLKEVV AKRQGVPADQ
41 LRVIFAGKEL RNDWTVQNCD LDQQSIVHIV QRPWRKGQEM
81 NATGGDDPRN AAGGCEREPQ SLTRVDLSSS VLPGDSVGLA
121 VILHTDSRKD SPPAGSPAGR SIYNSA YVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLK DRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARA EAASKET IKKTTKPCPR CHVPVEKNGG CMHMKCPQPQ
441 CRLEWCWNCG CEWNRVS MGD HWFDV

(SEQ ID NO: 14).

In some embodiments, the activated Parkin protein comprises a polypeptide sequence at least 75%, 80%, 85%, 90%, 92%, 93%, 94%, 95%, 96%, 97%, 98%, 99% or 100% identical to human Parkin F146A+N273K+W403A+C457S+F463A:

1 MIVFVRFNSS HGFPVEVDSD TSIFQLKEVV AKRQGVPADQ
41 LRVIFAGKEL RNDWTVQNCD LDQQSIVHIV QRPWRKGQEM
81 NATGGDDPRN AAGGCEREPQ SLTRVDLSSS VLPGDSVGLA
121 VILHTDSRKD SPPAGSPAGR SIYNSA YVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLK DRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARA EAASKET IKKTTKPCPR CHVPVEKNGG CMHMKCPQPQ
441 CRLEWCWNCG CEWNRVS MGD HWA DV

(SEQ ID NO: 15).

In some embodiments, the activated Parkin protein comprises a polypeptide sequence at least 75%, 80%, 85%, 90%, 92%, 93%, 94%, 95%, 96%, 97%, 98%, 99% or 100% identical to human Parkin N273K+W403A+F463A:

1 MIVFVRFNSS HGFPVEVDSD TSIFQLKEVV AKRQGVPADQ
41 LRVIFAGKEL RNDWTVQNCD LDQQSIVHIV QRPWRKGQEM
81 NATGGDDPRN AAGGCEREPQ SLTRVDLSSS VLPGDSVGLA
121 VILHTDSRKD SPPAGSPAGR SIYNSFYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLK DRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARA EAASKET IKKTTKPCPR CHVPVEKNGG CMHMKCPQPQ
441 CRLEWCWNCG CEWNRVCMGD HWA DV

(SEQ ID NO: 93).

In some embodiments, the activated Parkin protein comprises a polypeptide sequence at least 75%, 80%, 85%, 90%, 92%, 93%, 94%, 95%, 96%, 97%, 98%, 99% or 100% identical to human Parkin C431F:

1 MIVFVRFNSS HGFPVEVDSD TSIFQLKEVV AKRQGVPADQ
41 LRVIFAGKEL RNDWTVQNCD LDQQSIVHIV QRPWRKGQEM
81 NATGGDDPRN AAGGCEREPQ SLTRVDLSSS VLPGDSVGLA
121 VILHTDSRKD SPPAGSPAGR SIYNSFYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARWEAASKET IKKTTKPCPR CHVPVEKNGG FMHMKCPQPQ
441 CRLEWCWNCG CEWNRVCMGD HWFDV

(SEQ ID NO: 90).

The fusion protein comprising a Parkin protein or functional variant or fragment thereof. The Park protein may SEQ ID NO: 1 or another isoform of Parkin, e.g., having deletions and/or amino acid substitution(s) in an equivalent positions in a multiple sequence alignment of Parkin protein isoform, prepared, e.g., with ClustalW or MUSCLE alignment algorithms.

Further isoforms of Parkin include that may be used include the following, where the N-terminal portions in parentheses may optionally be deleted:

(MIVFVRFNSSHGFPVEVDSDTSIFQLKEVVAKRQGVPADQLRVIFAGKE
LRNDWTVQNCDLDQQSIVHIVQRPWR)KGQEMNATGGDDPRNAAGGCERE
PQSLTRVDLSSSVLPGDSVGLAVILHTDSRKDSPPAGSPAGR)SIYNSFY
VYCKGPCQRVQPGKLRVQCSTCRQATLTLTQEFFFKCGAHPTSDKETSVA
LHLIATNSRNITCITCTDVRSPVLVFQCNSRHVICLDCFHLYCVTRLNDR
QFVHDPQLGYSLPCVAGCPNSLIKELHHFRILGEEQYNRYQQYGAEECVL
QMGGVLCPRPGCGAGLLPEPDQRKVTCEGGNGLGCGFAFCRECKEAYHEG
ECSAVFEASGTTTQAYRVDERAAEQARWEAASKETIKKTTKPCPRCHVPV
EKNGGCMHMKCPQPQCRLEWCWNCGCEWNRVCMGDHWFDV (SEQ ID N
O: 2)

(MNATGGDDPRNAAGGCEREPQSLTRVDLSSSVLPGDSVGLAVILHTDSR
KDSPPAGSPAGR)SIYNSFYVYCKGPCQRVQPGKLRVQCSTCRQATLTLT
QGPSCWDDVLIPNRMSGECQSPHCPGTSAEFFFKCGAHPTSDKETSVALH
LIATNSRNITCITCTDVRSPVLVFQCNSRHVICLDCFHLYCVTRLNDRQF
VHDPQLGYSLPCWCLLPGM (SEQ ID NO: 3)

MSGECQSPHCPGTSAEFFFKCGAHPTSDKETSVALHLIATNSRNITCITC
TDVRSPVLVFQCNSRHVICLDCFHLYCVTRLNDRQFVHDPQLGYSLPCVA
GCPNSLIKELHHFRILGEEQYNRYQQYGAEECVLQMGGVLCPRPGCGAGL
LPEPDQRKVTCEGGNGLGCGFAFCRECKEAYHEGECSAVFEASGTTTQAY
RVDERAAEQARWEAASKETIKKTTKPCPRCHVPVEKNGGCMHMKCPQPQC
RLEWCWNCGCEWNRVCMGDHWFDV (SEQ ID NO: 4)

(MIVFVRFNSSHGFPVEVDSDTSIFQLKEVVAKRQGVPADQLRVIFAGKE
LRNDWTVQNCDLDQQSIVHIVQRPWR)KGQEMNATGGDDPRNAAGGCERE
PQSLTRVDLSSSVLPGDSVGLAVILHTDSRKDSPPAGSPAGR)SIYNSFY
VYCKGPCQRVQPGKLRVQCSTCRQATLTLTQGPSCWDDVLIPNRMSGECQ
SPHCPGTSAEFFFKCGAHPTSDKETSVALHLIATNSRNITCITCTDVRSP
VLVFQCNSRHVICLDCFHLYCVTRLNDRQFVHDPQLGYSLPCVGTGDTVV
LRGALGGFRRGVAGCPNSLIKELHHFRILGEEQYNRYQQYGAEECVLQMG
GVLCPRPGCGAGLLPEPDQRKVTCEGGNGLGCGYGQRRTK (SEQ ID N
O: 5)

(MIVFVRFNSSHGFPVEVDSDTSIFQLKEWAKRQGVPADQLRVIFAG)KE
LRNDWTVQEFFFKCGAHPTSDKETSVALHLIATNSRNITCITCTDVRSPV
LVFQCNSRHVICLDCFHLYCVTRLNDRQFVHDPQLGYSLPCVAGCPNSLI
KELHHFRILGEEQYNRYQQYGAEECVLQMGGVLCPRPGCGAGLLPEPDQR
KVTCEGGNGLGCGFAFCRECKEAYHEGECSAVFEASGTTTQAYRVDERAA
EQARWEAASKETIKKTTKPCPRCHVPVEKNGGCMHMKCPQPQCRLEWCWN
CGCEWNRVCMGDHWFDV (SEQ ID NO: 6)

(MIVFVRFNSSHGFPVEVDSDTSIFQLKEVVAKRQGVPADQLRVIFAGKE
LRNDWTVQNCDLDQQSIVHIVQRPWR)KGQEMNATGGDDPRNAAGGCERE
PQSLTRVDLSSSVLPGDSVGLAVILHTDSRKDSPPAGSPAGR)SIYNSFY
VYCKGPCQRVQPGKLRVQCSTCRQATLTLTQEFFFKCGAHPTSDKETSVA
LHLIATNSRNITCITCTDVRSPVLVFQCNSRHVICLDCFHLYCVTRLNDR
QFVHDPQLGYSLPCVAGCPNSLIKELHHFRILGEEQFAFCRECKEAYHEG
ECSAVFEASGTTTQAYRVDERAAEQARWEAASKETIKKTTKPCPRCHVPV
EKNGGCMHMKCPQPQCRLEWCWNCGCEWNRVCMGDHWFDV (SEQ ID N
O: 7)

(MIVFVRFNSSHGFPVEVDSDTSIFQLKEVVAKRQGVPADQLRVIFAGKE
LRNDWTVQNCDLDQQSIVHIVQRPWR)KGQEMNATGGDDPRNAAGGCERE
PQSLTRVDLSSSVLPGDSVGLAVILHTDSRKDSPPAGSPAGR)SIYNSFY
VYCKGPCQRVQPGKLRVQCSTCRQATLTLTQGPSCWDDVLIPNRMSGECQ
SPHCPGTSAEFFFKCGAHPTSDKETSVALHLIATNSRNITCITCTDVRSP
VLVFQCNSRHVICLDCFHLYCVTRLNDRQFVHDPQLGYSLPCVAGCPNSL
IKELHHFRILGEEQFAFCRECKEAYHEGECSAVFEASGTTTQAYRVDERA
AEQARWEAASKETIKKTTKPCPRCHVPVEKNGGCMHMKCPQPQCRLEWC
WNCGCEWNRVCMGDHWFDV (SEQ ID NO: 8)

The disclosure provides a fusion protein comprising a mitochondrial targeting sequence (MTS); a transmembrane domain (TMD); and a Parkin protein or functional variant or fragment thereof. The MTS may be the MTS of PINK1 or a functional variant thereof.

The MTS of PINK1 is post-translationally cleaved by mitochondrial processing peptidase (MPP) and Presenilins-associated rhomboid-like (PARL) protein. In some embodiments, the MTS, or another portion of the fusion protein comprises a mitochondrial processing peptidase (MPP) cleavage site. In some embodiments, the TMD comprises a PARL cleavage site. The MPP and PARL cleavage sites, when present, are cleaved when mitochondria are polarized. The present inventors have recognized that inclusion of these cleavage sites in the fusion protein may cause the fusion protein to be active specifically at damaged mitochondria.

The fusion protein may optionally have an amino acid substitution that stabilizes the product of PARL cleavage. For example, the fusion protein may comprises the amino acid substitution F104M, F104A, F104V, F104S, or F104G relative to a wild-type PINK1 sequence. An illustrative partial sequence of PINK1 is also follows:

1 MAVRQALGRG LQLGRALLLR FTGKPGRAYG LGRPGPAAGC
41 VRGERPGWAA GPGAEPRRVG LGLPNRLRFF RQSVAGLAAR
81 LQRQFVVRAW GCAGPCGRAV FLAF GLGLGL

(SEQ ID NO: 64).

With the F104M, F104A, F104V, F104S, F104G, or its functionally equivalent substitution at the same or different positions in PINK1, the fusion protein may be cleaved in the MTS by MPP and by PARL. Consequently the Parkin or Parkin fragment of the fusion protein is released in active form from the mitochondrial membrane. Advantageously, the Parkin fragment produced by the cleavage with PARL (at non-damaged mitochondria) may be released from the mitochondrial membrane into the cytoplasm in its active form.

The MTS may comprise a polypeptide sequence at least 95%, 96%, 97%, 98%, 99%, or 100% to residues 1-94 of human PINK1:

1 MAVRQALGRG LQLGRALLLR FTGKPGRAYG LGRPGPAAGC
41 VRGERPGWAA GPGAEPRRVG LGLPNRLRFF RQSVAGLAAR
81 LQRQFVVRAW GCAG

(SEQ ID NO: 65).

The MTS may be a minimal MTS. The MTS may comprise a polypeptide sequence at least 95%, 96%, 97%, 98%, 99%, or 100% identical to resides 1-34 of human PINK 1:

1 MAVRQALGRG LQLGRALLLR FTGKPGRAYG LGRP

(SEQ ID NO: 66).

The fusion proteins of the disclosure may further have a transmembrane domain (TMD). Suitable transmembrane domains may include any TMD capable of being cleaved by PARL.

In some embodiments, the TMD is the TMD of PINK1 or a functional variant thereof. The TMD may comprise a polypeptide sequence at least 95%, 96%, 97%, 98%, 99%, or 100% identical to residues 95-110 of human PINK1:

81 PCGRAV FLAFGLGLGL

(SEQ ID NO: 67).

In some embodiments, the TMD is the TMD of PINK1 or a functional variant thereof. The TMD may comprise a polypeptide sequence at least 95%, 96%, 97%, 98%, 99%, or 100% identical to residues 95-110 of human PINK1 F104M:

81 PCGRAV FLAM GLGLGL

(SEQ ID NO: 68).

In some embodiments, the TMD is the TMD of PINK1 or a functional variant thereof. The TMD may comprise a polypeptide sequence at least 95%, 96%, 97%, 98%, 99%, or 100% identical to residues 95-110 of human PINK1 F104A:

81 PCGRAV FLAA GLGLGL

(SEQ ID NO: 69).

In some embodiments, the fusion protein comprises the MTS of PINK1 and the TMD of PINK1—i.e. an MTS-TMD fragment of PINK1, or a functional variant thereof. In some embodiments, the fusion protein comprises an MTS-TMD fragment of PINK1 or a functional variant thereof, optionally comprising a polypeptide sequence at least 95%, 96%, 97%, 98%, 99%, or 100% identical to residues 1-110 of human PINK1:

1 MAVRQALGRG LQLGRALLLR FTGKPGRAYG LGRPGPAAGC
41 VRGERPGWAA GPGAEPRRVG LGLPNRLRFF RQSVAGLAAR
81 LQRQFVVRAW GCAGPCGRAV FLAFGLGLGL

(SEQ ID NO: 70).

The MTS-TMD fragment may comprises a polypeptide sequence identical to residues 1-110 of human PINK1 F104M:

1 MAVRQALGRG LQLGRALLLR FTGKPGRAYG LGRPGPAAGC
41 VRGERPGWAA GPGAEPRRVG LGLPNRLRFF RQSVAGLAAR
81 LQRQFVVRAW GCAGPCGRAV FLAM GLGLGL

(SEQ ID NO: 71).

The MTS-TMD fragment may comprises a polypeptide sequence identical to residues 1-110 of human PINK1 F104A:

1 MAVRQALGRG LQLGRALLLR FTGKPGRAYG LGRPGPAAGC
41 VRGERPGWAA GPGAEPRRVG LGLPNRLRFF RQSVAGLAAR
81 LQRQFVVRAW GCAGPCGRAV FLAA GLGLGL

(SEQ ID NO: 72).

In some cases the Parkin fragment is a fragment comprising a deletion of the N-terminal ubiquitin-like (Ubl) domain and optionally a deletion of the Ub1-RING0 interdomain linker. This fragment is termed herein a “ΔParkin protein.” The “ΔParkin protein” may optionally comprise one or more activating amino acid substitutions, such as F146A and/or W403A and/or C457S and/or F463A. Thus, in some embodiments, the fusion protein comprises a ΔParkin protein comprising a polypeptide sequence at least 95%, 96%, 97%, 98%, 99%, or 100% identical to residues 141-465 of human Parkin F146A+W403A:

121 SIYNSAYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARAEAASKET IKKTTKPCPR CHVPVEKNGG CMHMKCPQPQ
441 CRLEWCWNCG CEWNRVCMGD HWFDV

(SEQ ID NO: 73).

In some embodiments, the fusion protein comprises a ΔParkin protein comprising a polypeptide sequence at least 95%, 96%, 97%, 98%, 99%, or 100% identical to residues 76-465 of human Parkin F146A+W403A:

41 KGQEM
81 NATGGDDPRN AAGGCEREPQ SLTRVDLSSS VLPGDSVGLA
121 VILHTDSRKD SPPAGSPAGR SIYNSAYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARAEAASKET IKKTTKPCPR CHVPVEKNGG CMHMKCPQPQ
441 CRLEWCWNCG CEWNRVCMGD HWFDV

(SEQ ID NO: 74).

The full fusion protein of the disclosure may, in some embodiments, comprise the MTS-TMD of PINK1 C-terminally fused to a ΔParkin protein. Accordingly, in some embodiments, the fusion protein comprises a polypeptide sequence at least 95%, 96%, 97%, 98%, 99%, or 100% identical to the sequence:

1 MAVRQALGRG LQLGRALLLR FTGKPGRAYG LGRPGPAAGC
41 VRGERPGWAA GPGAEPRRVG LGLPNRLRFF RQSVAGLAAR
81 LQRQFVVRAW GCAGPCGRAV FLAFGLGLGL KGQEMNATGG
121 DDPRNAAGGC EREPQSLTRV DLSSSVLPGD SVGLAVILHT
161 DSRKDSPPAG SPAGRSIYNS AYVYCKGPCQ RVQPGKLRVQ
201 CSTCRQATLT LTQGPSCWDD VLIPNRMSGE CQSPHCPGTS
241 AEFFFKCGAH PTSDKETSVA LHLIATNSRN ITCITCTDVR
281 SPVLVFQCNS RHVICLDCFH LYCVTRLNDR QFVHDPQLGY
321 SLPCVAGCPN SLIKELHHFR ILGEEQYNRY QQYGAEECVL
361 QMGGVLCPRP GCGAGLLPEP DQRKVTCEGG NGLGCGFAFC
401 RECKEAYHEG ECSAVFEASG TTTQAYRVDE RAAEQARAEA
441 ASKETIKKTT KPCPRCHVPV EKNGGCMHMK CPQPQCRLEW
481 CWNCGCEWNR VCMGDHWFDV

(SEQ ID NO: 75).

In some embodiments, the fusion protein comprises a polypeptide sequence at least 95%, 96%, 97%, 98%, or 99% identical to the sequence:

1 MAVRQALGRG LQLGRALLLR FTGKPGRAYG LGRPGPAAGC
41 VRGERPGWAA GPGAEPRRVG LGLPNRLRFF RQSVAGLAAR
81 LQRQFVVRAW GCAGPCGRAV FLAF GLGLGL KGQEMNATGG
121 DDPRNAAGGC EREPQSLTRV DLSSSVLPGD SVGLAVILHT
161 DSRKDSPPAG SPAGRSIYNS AYVYCKGPCQ RVQPGKLRVQ
201 CSTCRQATLT LTQGPSCWDD VLIPNRMSGE CQSPHCPGTS
241 AEFFFKCGAH PTSDKETSVA LHLIATNSRN ITCITCTDVR
281 SPVLVFQCNS RHVICLDCFH LYCVTRLNDR QFVHDPQLGY
321 SLPCVAGCPN SLIKELHHFR ILGEEQYNRY QQYGAEECVL
361 QMGGVLCPRP GCGAGLLPEP DQRKVTCEGG NGLGCGFAFC
401 RECKEAYHEG ECSAVFEASG TTTQAYRVDE RAAEQARAEA
441 ASKETIKKTT KPCPRCHVPV EKNGGCMHMK CPQPQCRLEW
481 CWNCGCEWNR VCMGDHWFDV

(SEQ ID NO: 75).

where the sequence comprises an F104M or F104A substitution relative to a reference human PINK1 protein sequence of SEQ ID NO: 64.

In some embodiments, the fusion protein comprises a polypeptide sequence at least 95%, 96%, 97%, 98%, 99%, or 100% identical to the sequence:

1 MAVRQALGRG LQLGRALLLR FTGKPGRAYG LGRPGPAAGC
41 VRGERPGWAA GPGAEPRRVG LGLPNRLRFF RQSVAGLAAR
81 LQRQFVVRAW GCAGPCGRAV FLAFGLGLGL SIYNSAYVYC
121 KGPCQRVQPG KLRVQCSTCR QATLTLTQGP SCWDDVLIPN
161 RMSGECQSPH CPGTSAEFFF KCGAHPTSDK ETSVALHLIA
201 TNSRNITCIT CTDVRSPVLV FQCNSRHVIC LDCFHLYCVT
241 RLNDRQFVHD PQLGYSLPCV AGCPNSLIKE LHHFRILGEE
281 QYNRYQQYGA EECVLQMGGV LCPRPGCGAG LLPEPDQRKV
321 TCEGGNGLGC GFAFCRECKE AYHEGECSAV FEASGTTTQA
361 YRVDERAAEQ ARAEAASKET IKKTTKPCPR CHVPVEKNGG
401 CMHMKCPQPQ CRLEWCWNCG CEWNRVCMGD HWFDV

(SEQ ID NO: 76).

In some embodiments, the fusion protein comprises a polypeptide sequence at least 95%, 96%, 97%, 98%, or 99% identical to the sequence:

1 MAVRQALGRG LQLGRALLLR FTGKPGRAYG LGRPGPAAGC
41 VRGERPGWAA GPGAEPRRVG LGLPNRLRFF RQSVAGLAAR
81 LQRQFVVRAW GCAGPCGRAV FLAF GLGLGL SIYNSAYVYC
121 KGPCQRVQPG KLRVQCSTCR QATLTLTQGP SCWDDVLIPN
161 RMSGECQSPH CPGTSAEFFF KCGAHPTSDK ETSVALHLIA
201 TNSRNITCIT CTDVRSPVLV FQCNSRHVIC LDCFHLYCVT
241 RLNDRQFVHD PQLGYSLPCV AGCPNSLIKE LHHFRILGEE
281 QYNRYQQYGA EECVLQMGGV LCPRPGCGAG LLPEPDQRKV
321 TCEGGNGLGC GFAFCRECKE AYHEGECSAV FEASGTTTQA
361 YRVDERAAEQ ARAEAASKET IKKTTKPCPR CHVPVEKNGG
401 CMHMKCPQPQ CRLEWCWNCG CEWNRVCMGD HWFDV

(SEQ ID NO: 76). where the sequence comprises an F104M or F104A substitution relative to a reference human PINK1 protein sequence of SEQ ID NO: 64.

The full fusion protein of the disclosure may, in some embodiments, comprise the MTS-TMD of PINK1 C-terminally fused to a ΔParkin protein. Accordingly, in some embodiments, the fusion protein comprises a polypeptide sequence at least 95%, 96%, 97%, 98%, 99%, or 100% identical to the sequence:

1 MAVRQALGRG LQLGRALLLR FTGKPGRAYG LGRPGPAAGC
41 VRGERPGWAA GPGAEPRRVG LGLPNRLRFF RQSVAGLAAR
81 LQRQFVVRAW GCAGPCGRAV FLAFGLGLGL KGQEMNATGG
121 DDPRNAAGGC EREPQSLTRV DLSSSVLPGD SVGLAVILHT
161 DSRKDSPPAG SPAGRSIYNS FYVYCKGPCQ RVQPGKLRVQ
201 CSTCRQATLT LTQGPSCWDD VLIPNRMSGE CQSPHCPGTS
241 AEFFFKCGAH PTSDKETSVA LHLIATNSRN ITCITCTDVR
281 SPVLVFQCNS RHVICLDCFH LYCVTRLNDR QFVHDPQLGY
321 SLPCVAGCPN SLIKELHHFR ILGEEQYNRY QQYGAEECVL
361 QMGGVLCPRP GCGAGLLPEP DQRKVTCEGG NGLGCGFAFC
401 RECKEAYHEG ECSAVFEASG TTTQAYRVDE RAAEQARW EA
441 ASKETIKKTT KPCPRCHVPV EKNGGCMHMK CPQPQCRLEW
481 CWNCGCEWNR VCMGDHWA DV

(SEQ ID NO: 97). where the sequence comprises W403A and F463A substitutions.

The full fusion protein of the disclosure may, in some embodiments, comprise the MTS-TMD of PINK1 C-terminally fused to a ΔParkin protein. Accordingly, in some embodiments, the fusion protein comprises a polypeptide sequence at least 95%, 96%, 97%, 98%, 99%, or 100% identical to the sequence:

1 MAVRQALGRG LQLGRALLLR FTGKPGRAYG LGRPGPAAGC
41 VRGERPGWAA GPGAEPRRVG LGLPNRLRFF RQSVAGLAAR
81 LQRQFVVRAW GCAGPCGRAV FLAM GLGLGL KGQEMNATGG
121 DDPRNAAGGC EREPQSLTRV DLSSSVLPGD SVGLAVILHT
161 DSRKDSPPAG SPAGRSIYNS FYVYCKGPCQ RVQPGKLRVQ
201 CSTCRQATLT LTQGPSCWDD VLIPNRMSGE CQSPHCPGTS
241 AEFFFKCGAH PTSDKETSVA LHLIATNSRN ITCITCTDVR
281 SPVLVFQCNS RHVICLDCFH LYCVTRLNDR QFVHDPQLGY
321 SLPCVAGCPN SLIKELHHFR ILGEEQYNRY QQYGAEECVL
361 QMGGVLCPRP GCGAGLLPEP DQRKVTCEGG NGLGCGFAFC
401 RECKEAYHEG ECSAVFEASG TTTQAYRVDE RAAEQARW EA
441 ASKETIKKTT KPCPRCHVPV EKNGGCMHMK CPQPQCRLEW
481 CWNCGCEWNR VCMGDHWA DV

(SEQ ID NO: 99). where the sequence comprises F104M, W403A and F463A substitutions. The F104M is relative to a reference human PINK1 protein sequence of SEQ ID NO: 64; W403A is relative to a reference human Parkin protein sequence of SEQ ID NO: 1; and F463A is relative to a reference human Parkin protein sequence of SEQ ID NO: 1.

In some embodiments, the polynucleotide encoding the fusion protein comprises a polynucleotide sequence at least 75%, 80%, 85%, 90%, 92%, 93%, 94%, 95%, 96%, 97%, 98%, 99%, or 100% identical to SEQ ID NO: 77.

The polynucleotide sequence encoding the fusion protein may be codon-optimized. In some embodiments, the polynucleotide encoding the fusion protein comprises a polynucleotide sequence at least 75%, 80%, 85%, 90%, 92%, 93%, 94%, 95%, 96%, 97%, 98%, 99%, or 100% identical to SEQ ID NO: 78.

In some embodiments, the polynucleotide encoding the fusion protein comprises a polynucleotide sequence at least 75%, 80%, 85%, 90%, 92%, 93%, 94%, 95%, 96%, 97%, 98%, 99%, or 100% identical to SEQ ID NO: 79.

The polynucleotide sequence encoding the fusion protein may be codon-optimized. In some embodiments, the polynucleotide encoding the fusion protein comprises a polynucleotide sequence at least 75%, 80%, 85%, 90%, 92%, 93%, 94%, 95%, 96%, 97%, 98%, 99%, or 100% identical to SEQ ID NO: 80.

In some embodiments, the polynucleotide encoding the fusion protein comprises a polynucleotide sequence at least 75%, 80%, 85%, 90%, 92%, 93%, 94%, 95%, 96%, 97%, 98%, 99%, or 100% identical to SEQ ID NO: 81.

The polynucleotide sequence encoding the fusion protein may be codon-optimized. In some embodiments, the polynucleotide encoding the fusion protein comprises a polynucleotide sequence at least 75%, 80%, 85%, 90%, 92%, 93%, 94%, 95%, 96%, 97%, 98%, 99%, or 100% identical to SEQ ID NO: 82.

In some embodiments, the Parkin protein comprises a polypeptide sequence at least 75%, 80%, 85%, 90%, 92%, 93%, 94%, 95%, 96%, 97%, 98% or 99% identical to an isoform of human Parkin listed in Table 1, or a fragment thereof comprises a deletion of the portion(s) indicated in parentheses. In some embodiments, the Parkin protein comprises a polypeptide sequence at least 75%, 80%, 85%, 90%, 92%, 93%, 94%, 95%, 96%, 97%, 98% or 99% identical to the human Parkin of SEQ ID NO: 1 or a functional fragment thereof.

The Parkin may comprise a deletion of the ubiquitin-like (Ubl) domain of Parkin, or a deletion of a part of the Ubl domain. A Parkin having a deletion of the Ubl domain is termed herein “AParkin.” The boundaries of the Ubl domain may vary depending on the sequence of the reference Parkin. Generally, the Ubl domain of human Parkin is considered to be the first 75 amino-acid residues. Thus, in some embodiments, the Parkin protein is a ΔParkin protein comprising a deletion the ubiquitin-like (Ubl) domain, e.g., the ΔParkin comprises a deletion of residues 1-75, 5-75, 1-70, 5-75, or the like.

The activated Parkin may further comprise a deletion of the linker domain of Parkin (residues 76-140) or any portion of the linker.

In some embodiments, wherein the ΔParkin protein comprises a polypeptide sequence at least 75%, 80%, 85%, 90%, 92%, 93%, 94%, 95%, 96%, 97%, 98%, 99% or 100% identical to residues 76-465 of human Parkin

1
41 KGQEM
81 NATGGDDPRN AAGGCEREPQ SLTRVDLSSS VLPGDSVGLA
121 VILHTDSRKD SPPAGSPAGR SIYNSFYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARWEAASKET IKKTTKPCPR CHVPVEKNGG CMHMKCPQPQ
441 CRLEWCWNCG CEWNRVCMGD HWFDV

(SEQ ID NO: 16).

In some embodiments, wherein the ΔParkin protein comprises a polypeptide sequence at least 75%, 80%, 85%, 90%, 92%, 93%, 94%, 95%, 96%, 97%, 98%, 99% or 100% identical to residues 141-465 of human Parkin:

1
41
81
121 SIYNSFYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARWEAASKET IKKTTKPCPR CHVPVEKNGG CMHMKCPQPQ
441 CRLEWCWNCG CEWNRVCMGD HWFDV

(SEQ ID NO: 17).

In some embodiments, wherein the ΔParkin protein comprises a polypeptide sequence at least 75%, 80%, 85%, 90%, 92%, 93%, 94%, 95%, 96%, 97%, 98%, 99% or 100% identical to residues 76-465 of human Parkin F146A+W403A:

1
41 KGQEM
81 NATGGDDPRN AAGGCEREPQ SLTRVDLSSS VLPGDSVGLA
121 VILHTDSRKD SPPAGSPAGR SIYNSA YVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARA EAASKET IKKTTKPCPR CHVPVEKNGG CMHMKCPQPQ
441 CRLEWCWNCG CEWNRVCMGD HWFDV

(SEQ ID NO: 18).

In some embodiments, wherein the ΔParkin protein comprises a polypeptide sequence at least 75%, 80%, 85%, 90%, 92%, 93%, 94%, 95%, 96%, 97%, 98%, 99% or 100% identical to residues 141-465 of human Parkin W403A + F463A:

1
41
81
121 SIYNSFYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARA EAASKET IKKTTKPCPR CHVPVEKNGG CMHMKCPQPQ
441 CRLEWCWNCG CEWNRVCMGD HWA DV

(SEQ ID NO: 95).

In some embodiments, the activated ΔParkin protein consists of the polypeptide sequence of residues 76-465 of human Parkin (SEQ ID NO: 16). In some embodiments, the activated ΔParkin protein consists of the polypeptide sequence of residues 76-465 of human Parkin F146A+W403A (SEQ ID NO: 18).

In some embodiments, the activated ΔParkin protein consists of a polypeptide sequence identical, across the full length of the polypeptide sequence, to a portion of residues 76-465 of human Parkin (SEQ ID NO: 16), the polypeptide sequence having C-terminal and/or N-terminal truncations of 1, 2, 3, 4, 5, 6, 7, 8, 9, or 10 amino acids with respect to SEQ ID NO: 16.

In some embodiments, the activated ΔParkin protein consists of a polypeptide sequence identical, across the full length of the polypeptide sequence, to a portion of residues 76-465 of human Parkin F146A+W403A (SEQ ID NO: 18), the polypeptide sequence having C-terminal and/or N-terminal truncations of 1, 2, 3, 4, 5, 6, 7, 8, 9, or 10 amino acids with respect to SEQ ID NO: 18.

In some embodiments, the activated Parkin protein comprises a polypeptide sequence at least 75%, 80%, 85%, 90%, 92%, 93%, 94%, 95%, 96%, 97%, 98%, 99% or 100% identical to residues 76-465 (or residues 141-465) of human Parkin N273K+W403A+C457S:

1
41 KGQEM
81 NATGGDDPRN AAGGCEREPQ SLTRVDLSSS VLPGDSVGLA
121 VILHTDSRKD SPPAGSPAGR SIYNSFYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLK DRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARA EAASKET IKKTTKPCPR CHVPVEKNGG CMHMKCPQPQ
441 CRLEWCWNCG CEWNRVS MGD HWFDV

In some embodiments, the activated Parkin protein comprises a polypeptide sequence at least 75%, 80%, 85%, 90%, 92%, 93%, 94%, 95%, 96%, 97%, 98%, 99% or 100% identical to residues 76-465 (or residues 141-465) of human Parkin N273K+W403A+F463A:

1
41 KGQEM
81 NATGGDDPRN AAGGCEREPQ SLTRVDLSSS VLPGDSVGLA
121 VILHTDSRKD SPPAGSPAGR SIYNSFYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLK DRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARA EAASKET IKKTTKPCPR CHVPVEKNGG CMHMKCPQPQ
441 CRLEWCWNCG CEWNRVCMGD HWA DV

(SEQ ID NO: 19).

In some embodiments, the activated Parkin protein comprises a polypeptide sequence at least 75%, 80%, 85%, 90%, 92%, 93%, 94%, 95%, 96%, 97%, 98%, 99% or 100% identical to residues 76-465 (or residues 141-465) of human Parkin F146A+N273K+W403A+C457S:

1
41 KGQEM
81 NATGGDDPRN AAGGCEREPQ SLTRVDLSSS VLPGDSVGLA
121 VILHTDSRKD SPPAGSPAGR SIYNSA YVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLK DRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARA EAASKET IKKTTKPCPR CHVPVEKNGG CMHMKCPQPQ
441 CRLEWCWNCG CEWNRVS MGD HWFDV

(SEQ ID NO: 20).

In some embodiments, the activated Parkin protein comprises a polypeptide sequence at least 75%, 80%, 85%, 90%, 92%, 93%, 94%, 95%, 96%, 97%, 98%, 99% or 100% identical to residues 76-465 (or residues 141-465) of human Parkin F 146A+N273K+W403A+C457S+F463A:

1
41 KGQEM
81 NATGGDDPRN AAGGCEREPQ SLTRVDLSSS VLPGDSVGLA
121 VILHTDSRKD SPPAGSPAGR SIYNSA YVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLK DRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARA EAASKET IKKTTKPCPR CHVPVEKNGG CMHMKCPQPQ
441 CRLEWCWNCG CEWNRVS MGD HWA DV

(SEQ ID NO: 21).

In some embodiments, the polynucleotide encoding the ΔParkin comprises a polynucleotide sequence at least 75%, 80%, 85%, 90%, 92%, 93%, 94%, 95%, 96%, 97%, 98%, 99%, or 100% identical to SEQ ID NO: 22.

In some embodiments, the polynucleotide encoding the ΔParkin comprises a polynucleotide sequence at least 75%, 80%, 85%, 90%, 92%, 93%, 94%, 95%, 96%, 97%, 98%, 99%, or 100% identical to SEQ ID NO: 23.

The polynucleotide encoding the ΔParkin protein may be codon-optimized. In some embodiments, the polynucleotide encoding the ΔParkin protein comprises a polynucleotide sequence at least 75%, 80%, 85%, 90%, 92%, 93%, 94%, 95%, 96%, 97%, 98%, 99%, or 100% identical to SEQ ID NO: 24.

In some embodiments, the polynucleotide encoding the ΔParkin protein comprises a polynucleotide sequence at least 75%, 80%, 85%, 90%, 92%, 93%, 94%, 95%, 96%, 97%, 98%, 99%, or 100% identical to SEQ ID NO: 25.

### Vector Genome

The AAV virions of the disclosure comprise a vector genome. The vector genome may comprise an expression cassette (or a polynucleotide cassette for gene-editing applications not requiring expression of the polynucleotide sequence). Any suitable inverted terminal repeats (ITRs) may be used. The ITRs may be from the same serotype as the capsid or a different serotype (e.g., AAV2 ITRs may be used).

In some embodiments, the 5′ ITR comprises a polynucleotide sequence at least 75%, 80%, 85%, 90%, 92%, 93%, 94%, 95%, 96%, 97%, 98%, 99%, or 100% identical to SEQ ID NO: 26.

In some embodiments, the 5′ ITR comprises a polynucleotide sequence at least 75%, 80%, 85%, 90%, 92%, 93%, 94%, 95%, 96%, 97%, 98%, 99%, or 100% identical to SEQ ID NO: 27.

In some embodiments the vector genome comprises one or more filler sequences, e.g., at least 75%, 80%, 85%, 90%, 92%, 93%, 94%, 95%, 96%, 97%, 98%, 99%, or 100% identical to SEQ ID NO: 28.

### Promoters

In some embodiments, the polynucleotide sequence encoding a Parkin protein, e.g., an activated Parkin protein, or functional variant or fragment thereof is operably linked to a promoter.

The present disclosure contemplates use of various promoters. Promoters useful in embodiments of the present disclosure include, without limitation, a cytomegalovirus (CMV) promoter, phosphoglycerate kinase (PGK) promoter, or a promoter sequence comprised of the CMV enhancer and portions of the chicken beta-actin promoter and the rabbit beta-globin gene (CAG). In some cases, the promoter may be a synthetic promoter. Exemplary synthetic promoters are provided by Schlabach et al. PNAS USA. 107(6):2538-43 (2010).

In some embodiments, a polynucleotide sequence encoding a Parkin protein, or functional variant or fragment thereof, is operatively linked to an inducible promoter. An inducible promoter may be configured to cause the polynucleotide sequence to be transcriptionally expressed or not transcriptionally expressed in response to addition or accumulation of an agent or in response to removal, degradation, or dilution of an agent. The agent may be a drug. The agent may be tetracycline or one of its derivatives, including, without limitation, doxycycline. In some cases, the inducible promoter is a tet-on promoter, a tet-off promoter, a chemically-regulated promoter, a physically-regulated promoter (i.e., a promoter that responds to presence or absence of light or to low or high temperature). Inducible promoters include heavy metal ion inducible promoters (such as the mouse mammary tumor virus (mMTV) promoter or various growth hormone promoters), and the promoters from T7 phage which are active in the presence of T7 RNA polymerase. This list of inducible promoters is non-limiting.

In some cases, the promoter is a tissue-specific promoter, such as a promoter capable of driving expression in a neuron to a greater extent than in a non-neuronal cell. In some embodiments, tissue-specific promoter is a selected from any various neuron-specific promoters including but not limited to hSYNl (human synapsin), INA (alpha-internexin), NES (nestin), TH (tyrosine hydroxylase), FOXA2 (Forkhead box A2), CaMKII (calmodulin-dependent protein kinase II), and NSE (neuron-specific enolase). In some cases, the promoter is a ubiquitous promoter. A “ubiquitous promoter” refers to a promoter that is not tissue-specific under experimental or clinical conditions. In some cases, the ubiquitous promoter is any one of CMV, CAG, UBC, PGK, EF1-alpha, GAPDH, SV40, HBV, chicken beta-actin, and human beta-actin promoters.

In some embodiments, the promoter sequence is selected from Table 3, and sequences having at least 95%, at least 98%, or least 99% identity thereto.

TABLE 3

| PROMOTER | SEQ ID NO: |
|---|---|
| Human beta-actin (HuBa) | 29 |
| Chicken beta-actin (CBA) | 30 |
| Cytomegalovirus (CMV) | 31 |
| Human EF1-alpha (EF1-α) | 32 |
| Human Synapsin1 (Syn) | 33 |
| Human CamKIIa (CaMKIIa) | 34 |

Further illustrative examples of promoters are the SV40 late promoter from simian virus 40, the Baculovirus polyhedron enhancer/promoter element, Herpes Simplex Virus thymidine kinase (HSV tk), the immediate early promoter from cytomegalovirus (CMV) and various retroviral promoters including LTR elements. A large variety of other promoters are known and generally available in the art, and the sequences of many such promoters are available in sequence databases such as the GenBank database.

### Other Regulatory Elements

In some cases, vectors of the present disclosure further comprise one or more regulatory elements selected from the group consisting of an enhancer, an intron, a poly-A signal, a 2A peptide encoding sequence, a WPRE (Woodchuck hepatitis virus posttranscriptional regulatory element), and a HPRE (Hepatitis B posttranscriptional regulatory element).

In some embodiments, the vector comprises a CMV enhancer.

In certain embodiments, the vectors comprise one or more enhancers. In particular embodiments, the enhancer is a CMV enhancer sequence, a GAPDH enhancer sequence, a (β-actin enhancer sequence, or an EF1-α enhancer sequence. Sequences of the foregoing are known in the art. For example, the sequence of the CMV immediate early (IE) enhancer is SEQ ID NO: 35.

In certain embodiments, the vectors comprise one or more introns. In particular embodiments, the intron is a rabbit globin intron sequence, a chicken β-actin intron sequence, a synthetic intron sequence, or an EF1-α intron sequence.

In certain embodiments, the vectors comprise a polyA sequence. In particular embodiments, the polyA sequence is a rabbit globin polyA sequence, a human growth hormone polyA sequence, a bovine growth hormone polyA sequence, a PGK polyA sequence, an SV40 polyA sequence, or a TK polyA sequence. In some embodiments, the poly-A signal may be a bovine growth hormone polyadenylation signal (bGHpA).

In certain embodiments, the vectors comprise one or more transcript stabilizing element. In particular embodiments, the transcript stabilizing element is a WPRE sequence, a HPRE sequence, a scaffold-attachment region, a 3′ UTR, or a 5′ UTR. In particular embodiments, the vectors comprise both a 5′ UTR and a 3′ UTR.

In some embodiments, the vector comprises a 5′ untranslated region (UTR) selected from Table 4.

TABLE 4

| 5′ UNTRANSLATED REGION | SEQ ID NO: |
|---|---|
| Human beta-actin exon/intron | 36 |
| Chicken beta-actin exon/intron + rabbit globin intron | 37 |
| 5′ UTR-Syn1 Hs | 38 |
| CMV IE exon | 39 |
| TPL-eMLP (adenovirus derived enhancer element) | 40 |
| Human EF1-α intron/exon | 41 |
| 5′ UTR human CamKIIa | 42 |

In some embodiments, the vector comprises a 3′ untranslated region selected from Table 5.

TABLE 5

| 3′ UNTRANSLATED REGION | SEQ ID NO: |
|---|---|
| WPRE(x) (mutated woodchuck hepatitis regulatory element) | 43 |
| CAAX | 44 |
| EES | 45 |
| HPRE | 46 |
| R2V17 (HepB derived enhancer element) | 47 |
| 3′UTR(globin) | 48 |
| WPRE(r) | 49 |

In some embodiments, the vector comprises a polyadenylation sequence (polyA) selected from Table 6.

TABLE 6

| POLY-ADENYLATION SITE | SEQ ID NO: |
|---|---|
| Rabbit globin (pAGlobin-Oc) | 50 |
| Bovine growth hormone (pAGH-Bt) | 51 |
| Human growth hormone (pAGH-Hs) | 52 |

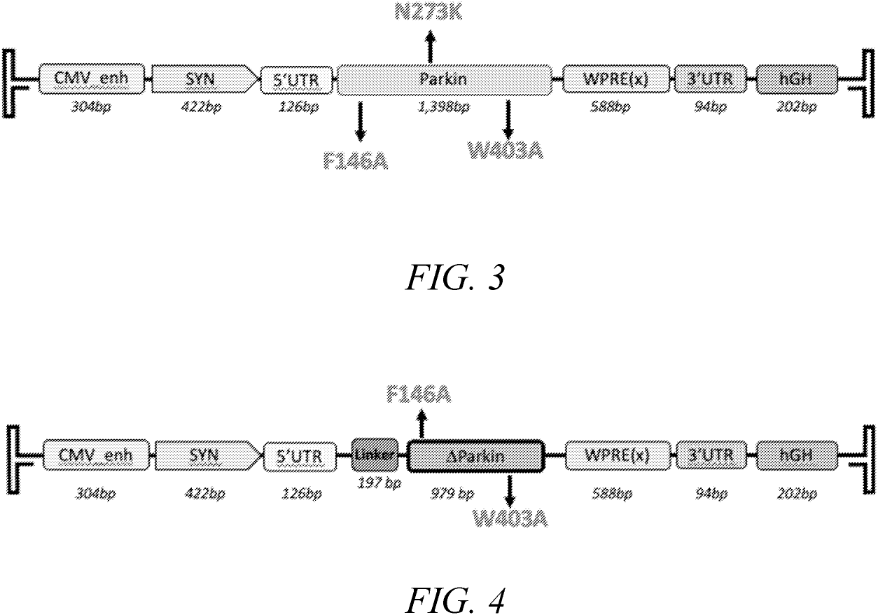

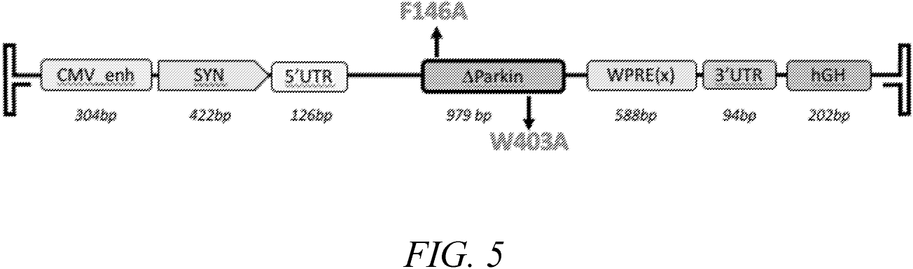

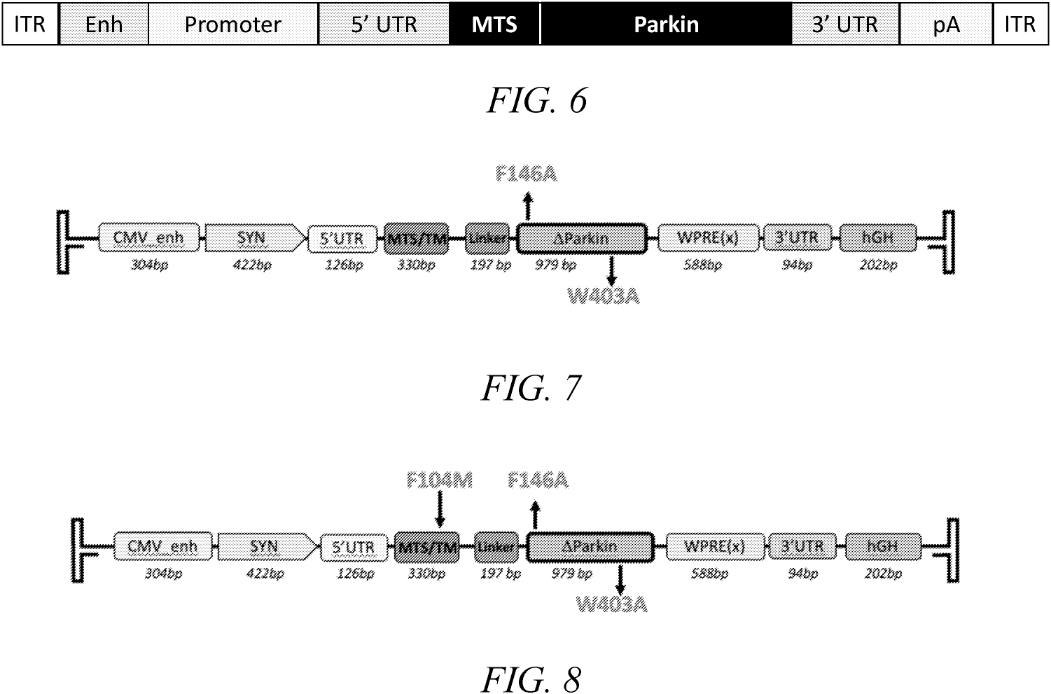

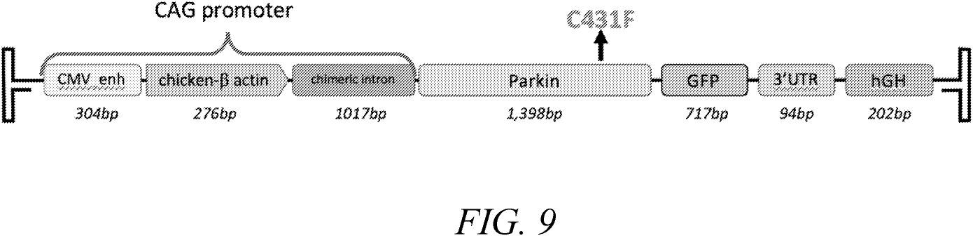

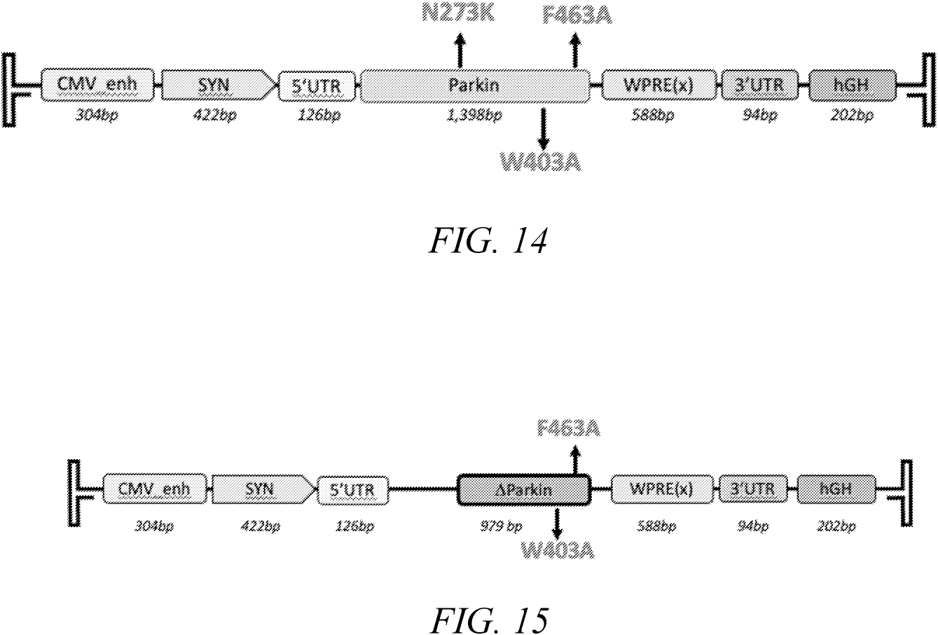

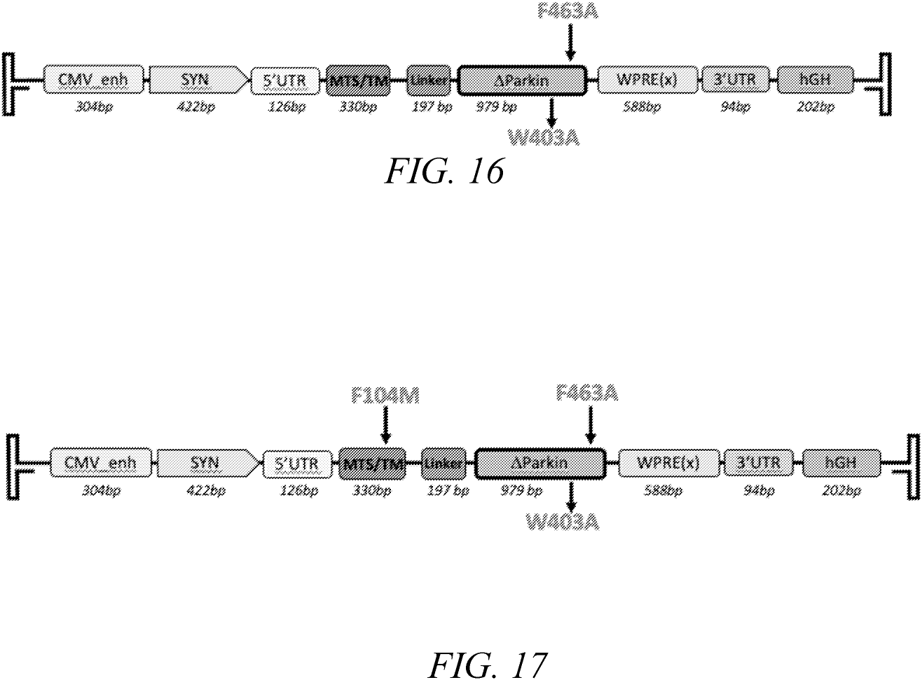

Illustrative vector genomes are depicted in FIGS. 2-5, 6-8, and 14-17 provided as SEQ ID NOs: 53-58, 83-88, 91, 92, 94, 96, and 98. In some embodiments, the vector genome comprises, consists essentially of, or consists of a polynucleotide sequence that shares at least 90%, 91%, 92%, 93%, 94%, 95%, 96%, 97%, 98%, 99%, or 100% identity to any one of SEQ ID NOs: 53-58,83-88, 91, 92, 94, 96, and 98.

In an embodiment, the expression cassette comprises, in 5′ to 3′ order, HuBA promoter, the polynucleotide sequence encoding the activated Parkin, WPRE(x), and pAGlobin-Oc.

In an embodiment, the expression cassette comprises, in 5′ to 3′ order, CMV promoter, TPL-eMLP enhancer, the polynucleotide sequence encoding the activated Parkin, WPRE(r), and pAGlobin-Oc.

In an embodiment, the expression cassette comprises, in 5′ to 3′ order, Syn promoter, the polynucleotide sequence encoding the activated Parkin, WPRE(r), 3′UTR (globin), and pAGH-Bt.

In an embodiment, the expression cassette comprises, in 5′ to 3′ order, CBA promoter, the polynucleotide sequence encoding the activated Parkin, and pAGH-Bt.

In an embodiment, the expression cassette comprises, in 5′ to 3′ order, EF1α promoter, the polynucleotide sequence encoding the activated Parkin, and pAGlobin-Oc.

In an embodiment, the expression cassette comprises, in 5′ to 3′ order, HuBA promoter, the polynucleotide sequence encoding the activated Parkin, R2V17, and pAGH-Bt.

In an embodiment, the expression cassette comprises, in 5′ to 3′ order, Syn promoter, the polynucleotide sequence encoding the activated Parkin, WPRE(x), 3′UTR (globin), and pAGH-Hs.

In an embodiment, the expression cassette comprises, in 5′ to 3′ order, CaMKIIa promoter, the polynucleotide sequence encoding the activated Parkin, WPRE(r), and pAGH-Hs.

In an embodiment, the expression cassette comprises, in 5′ to 3′ order, CMV promoter, TPL-eMLP enhancer, the polynucleotide sequence encoding the activated Parkin, WPRE(r), and pAGH-Hs.

In an embodiment, the expression cassette comprises, in 5′ to 3′ order, HuBA promoter, the polynucleotide sequence encoding the activated Parkin, and pAGH-Hs.

In an embodiment, the expression cassette comprises, in 5′ to 3′ order, CMV promoter, TPL/eMLP enhancer, the polynucleotide sequence encoding the activated Parkin, R2V17, 3′UTR (globin), and pAGH-Bt.

In an embodiment, the expression cassette comprises, in 5′ to 3′ order, EF1α promoter, the polynucleotide sequence encoding the activated Parkin, WPRE(r), and pAGH-Bt.

In an embodiment, the expression cassette comprises, in 5′ to 3′ order, Syn promoter, the polynucleotide sequence encoding the activated Parkin, R2V17, and pAGlobin-Oc.

In an embodiment, the expression cassette comprises, in 5′ to 3′ order, CaMKIIa promoter, the polynucleotide sequence encoding the activated Parkin, R2V17, and pAGlobin-Oc.

In an embodiment, the expression cassette comprises, in 5′ to 3′ order, CBA promoter, the polynucleotide sequence encoding the activated Parkin, WPRE(x), 3′UTR (globin), and pAGH-Hs.

In an embodiment, the expression cassette comprises, in 5′ to 3′ order, CBA promoter, the polynucleotide sequence encoding the activated Parkin, 3′UTR (globin), and pAGlobin-Oc.

In an embodiment, the expression cassette comprises, in 5′ to 3′ order, CaMKIIa promoter, the polynucleotide sequence encoding the activated Parkin, R2V17, and pAGH-Bt.

In an embodiment, the expression cassette comprises, in 5′ to 3′ order, EF1α promoter, the polynucleotide sequence encoding the activated Parkin, R2V17, 3′UTR (globin), and pAGH-Hs.

In an embodiment, the expression cassette comprises, in 5′ to 3′ order, CMV promoter, the polynucleotide sequence encoding the activated Parkin, R2V17, 3′UTR (globin), and pAGH-Hs.

In an embodiment, the expression cassette comprises, in 5′ to 3′ order, CMV promoter, the polynucleotide sequence encoding the activated Parkin, and pAGH-Hs.

In embodiments of the foregoing, the order of the elements 5′ to the polynucleotide sequence encoding the activated Parkin are reversed so that the promoter precedes the enhancer elements or the enhancer element precedes the promoter element.

### Adeno-Associated Virus Vector

Adeno-associated virus (AAV) is a replication-deficient parvovirus, the single-stranded DNA genome of which is about 4.7 kb in length including two 145-nucleotide inverted terminal repeat (ITRs). There are multiple known variants of AAV, also sometimes called serotypes when classified by antigenic epitopes. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_{_}001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAVrh.74 genome is provided in U.S. Pat. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep78, rep68, rep52, and rep40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

AAV DNA in the rAAV genomes may be from any AAV variant or serotype for which a recombinant virus can be derived including, but not limited to, AAV variants or serotypes AAV-1, AAV-2, AAV-3, AAV-4, AAV-5, AAV-6, AAV-7, AAV-8, AAV-9, AAV- 10, AAV-11, AAV- 12, AAV-13 and AAVrh10. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). The nucleotide sequences of the genomes of various AAV serotypes are known in the art.

In some cases, the rAAV comprises a self-complementary genome. As defined herein, an rAAV comprising a “self-complementary” or “double stranded” genome refers to an rAAV which has been engineered such that the coding region of the rAAV is configured to form an intra-molecular double-stranded DNA template, as described in McCarty et al. Self-complementary recombinant adeno-associated virus (scAAV) vectors promote efficient transduction independently of DNA synthesis. Gene Therapy. 8 (16): 1248-54 (2001). The present disclosure contemplates the use, in some cases, of an rAAV comprising a self-complementary genome because upon infection (such transduction), rather than waiting for cell mediated synthesis of the second strand of the rAAV genome, the two complementary halves of scAAV will associate to form one double stranded DNA (dsDNA) unit that is ready for immediate replication and transcription. It will be understood that instead of the full coding capacity found in rAAV (4.7-6 kb), rAAV comprising a self-complementary genome can only hold about half of that amount (≈2.4kb).

In other cases, the rAAV vector comprises a single stranded genome. As defined herein, a “single standard” genome refers to a genome that is not self-complementary. In most cases, non-recombinant AAVs have singled stranded DNA genomes. There have been some indications that rAAVs should be scAAVs to achieve efficient transduction of cells. The present disclosure contemplates, however, rAAV vectors that maybe have singled stranded genomes, rather than self-complementary genomes, with the understanding that other genetic modifications of the rAAV vector may be beneficial to obtain optimal gene transcription in target cells. In some cases, the present disclosure relates to single-stranded rAAV vectors capable of achieving efficient gene transfer to anterior segment in the mouse eye. See Wang et al. Single stranded adeno-associated virus achieves efficient gene transfer to anterior segment in the mouse eye. PLoS ONE 12(8): e0182473 (2017).

In some cases, the rAAV vector is of the serotype AAV1, AAV2, AAV4, AAV5, AAV6, AAV7, AAV8, AAV9, AAV10, AAV11, AAV12, AAV13, AAVrh10, or AAVrh74. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). In some cases, the rAAV vector is of the serotype AAV9. In some embodiments, said rAAV vector is of serotype AAV9 and comprises a single stranded genome. In some embodiments, said rAAV vector is of serotype AAV9 and comprises a self-complementary genome. In some embodiments, a rAAV vector comprises the inverted terminal repeat (ITR) sequences of AAV2. In some embodiments, the rAAV vector comprises an AAV2 genome, such that the rAAV vector is an AAV-2/9 vector, an AAV-2/6 vector, or an AAV-2/8 vector.

Full-length sequences and sequences for capsid genes for most known AAVs are provided in U.S. Pat. No. 8,524,446, which is incorporated herein in its entirety.

AAV vectors may comprise wild-type AAV sequence or they may comprise one or more modifications to a wild-type AAV sequence. In certain embodiments, an AAV vector comprises one or more amino acid modifications, e.g., substitutions, deletions, or insertions, within a capsid protein, e.g., VP1, VP2 and/or VP3. In particular embodiments, the modification provides for reduced immunogenicity when the AAV vector is provided to a subj ect.

Capsid proteins of a rAAV may be modified so that the rAAV is targeted to a particular target tissue of interest such as neurons or more particularly a dopaminergic neuron. See, for example, Albert et al. AAV Vector-Mediated Gene Delivery to Substantia Nigra Dopamine Neurons: Implications for Gene Therapy and Disease Models. Genes. 2017 Feb 8; see also U.S. Pat. No. 6,180,613 and U.S. Pat. Pub. No. US20120082650A1, the disclosures of both of which are incorporated by reference herein. In some embodiments, the rAAV is directly injected into the substantia nigra of the subject.

In some embodiments, the rAAV virion is an AAV2 rAAV virion. The capsid many be an AAV2 capsid or functional variant thereof. In some embodiments, the AAV2 capsid shares at least 98%, 99%, or 100% identity to a reference AAV2 capsid, e.g. SEQ ID NO: 59.

In some embodiments, the rAAV virion is an AAV9 rAAV virion. The capsid many be an AAV9 capsid or functional variant thereof. In some embodiments, the AAV9 capsid shares at least 98%, 99%, or 100% identity to a reference AAV9 capsid, e.g., SEQ ID NO: 60.

In some embodiments, the rAAV virion is an AAV-PHP.B rAAV virion or a neutrotrophic variant thereof, such as, without limitation, those disclosed in Int′1 Pat. Pub. Nos. WO 2015/038958 A1 and WO 2017/100671 A1. For example, the AAV capsid may comprise at least 4 contiguous amino acids from the sequence TLAVPFK (SEQ ID NO:62) or KFPVALT (SEQ ID NO:63), e.g., inserted between a sequence encoding for amino acids 588 and 589 of AAV9.

The capsid many be an AAV-PHP.B capsid or functional variant thereof. In some embodiments, the AAV-PHP.B capsid shares at least 98%, 99%, or 100% identity to a reference AAV-PHP.B capsid, e.g., SEQ ID NO: 61.

Further AAV capsids used in the rAAV virions of the disclosure include those disclosed in Pat. Pub. Nos. WO 2009/012176 A2 and WO 2015/168666 A2.

In certain embodiments, the disclosure provides an rAAV viron, e.g., an AAV2 rAAV viron or an AAV9 rAAV viron, comprising an expression cassette disclosed herein.

In particular embodiments of the rAAV viron, e.g., AAV2 rAAV viron or AAV9 rAAV viron, the expression cassette comprises, in 5′ to 3′ order, HuBA promoter, the polynucleotide sequence encoding the activated Parkin, WPRE(x), and pAGlobin-Oc.

In particular embodiments of the rAAV viron, e.g., AAV2 rAAV viron or AAV9 rAAV viron, the expression cassette comprises, in 5′ to 3′ order, CMV promoter, TPL-eMLP enhancer, the polynucleotide sequence encoding the activated Parkin, WPRE(r), and pAGlobin-Oc.

In particular embodiments of the rAAV viron, e.g., AAV2 rAAV viron or AAV9 rAAV viron, the expression cassette comprises, in 5′ to 3′ order, Syn promoter, the polynucleotide sequence encoding the activated Parkin, WPRE(r), 3′UTR (globin), and pAGH-Bt.

In particular embodiments of the rAAV viron, e.g., AAV2 rAAV viron or AAV9 rAAV viron, the expression cassette comprises, in 5′ to 3′ order, CBA promoter, the polynucleotide sequence encoding the activated Parkin, and pAGH-Bt.

In particular embodiments of the rAAV viron, e.g., AAV2 rAAV viron or AAV9 rAAV viron, the expression cassette comprises, in 5′ to 3′ order, EF1α promoter, the polynucleotide sequence encoding the activated Parkin, and pAGlobin-Oc.

In particular embodiments of the rAAV viron, e.g., AAV2 rAAV viron or AAV9 rAAV viron, the expression cassette comprises, in 5′ to 3′ order, HuBA promoter, the polynucleotide sequence encoding the activated Parkin, R2V17, and pAGH-Bt.

In particular embodiments of the rAAV viron, e.g., AAV2 rAAV viron or AAV9 rAAV viron, the expression cassette comprises, in 5′ to 3′ order, Syn promoter, the polynucleotide sequence encoding the activated Parkin, WPRE(x), 3′UTR (globin), and pAGH-Hs.

In particular embodiments of the rAAV viron, e.g., AAV2 rAAV viron or AAV9 rAAV viron, the expression cassette comprises, in 5′ to 3′ order, CaMKIIa promoter, the polynucleotide sequence encoding the activated Parkin, WPRE(r), and pAGH-Hs.

In particular embodiments of the rAAV viron, e.g., AAV2 rAAV viron or AAV9 rAAV viron, the expression cassette comprises, in 5′ to 3′ order, CMV promoter, TPL-eMLP enhancer, the polynucleotide sequence encoding the activated Parkin, WPRE(r), and pAGH-Hs.

In particular embodiments of the rAAV viron, e.g., AAV2 rAAV viron or AAV9 rAAV viron, the expression cassette comprises, in 5′ to 3′ order, HuBA promoter, the polynucleotide sequence encoding the activated Parkin, and pAGH-Hs.

In particular embodiments of the rAAV viron, e.g., AAV2 rAAV viron or AAV9 rAAV viron, the expression cassette comprises, in 5′ to 3′ order, CMV promoter, TPL/eMLP enhancer, the polynucleotide sequence encoding the activated Parkin, R2V17, 3′UTR (globin), and pAGH-Bt.

In particular embodiments of the rAAV viron, e.g., AAV2 rAAV viron or AAV9 rAAV viron, the expression cassette comprises, in 5′ to 3′ order, EF1α promoter, the polynucleotide sequence encoding the activated Parkin, WPRE(r), and pAGH-Bt.

In particular embodiments of the rAAV viron, e.g., AAV2 rAAV viron or AAV9 rAAV viron, the expression cassette comprises, in 5′ to 3′ order, Syn promoter, the polynucleotide sequence encoding the activated Parkin, R2V17, and pAGlobin-Oc.

In particular embodiments of the rAAV viron, e.g., AAV2 rAAV viron or AAV9 rAAV viron, the expression cassette comprises, in 5′ to 3′ order, CaMKIIa promoter, the polynucleotide sequence encoding the activated Parkin, R2V17, and pAGlobin-Oc.

In particular embodiments of the rAAV viron, e.g., AAV2 rAAV viron or AAV9 rAAV viron, the expression cassette comprises, in 5′ to 3′ order, CBA promoter, the polynucleotide sequence encoding the activated Parkin, WPRE(x), 3′UTR (globin), and pAGH-Hs.

In particular embodiments of the rAAV viron, e.g., AAV2 rAAV viron or AAV9 rAAV viron, the expression cassette comprises, in 5′ to 3′ order, CBA promoter, the polynucleotide sequence encoding the activated Parkin, 3′UTR (globin), and pAGlobin-Oc.

In particular embodiments of the rAAV viron, e.g., AAV2 rAAV viron or AAV9 rAAV viron, the expression cassette comprises, in 5′ to 3′ order, CaMKIIa promoter, the polynucleotide sequence encoding the activated Parkin, R2V17, and pAGH-Bt.

In particular embodiments of the rAAV viron, e.g., AAV2 rAAV viron or AAV9 rAAV viron, the expression cassette comprises, in 5′ to 3′ order, EF1α promoter, the polynucleotide sequence encoding the activated Parkin, R2V17, 3′UTR (globin), and pAGH-Hs.

In particular embodiments of the rAAV viron, e.g., AAV2 rAAV viron or AAV9 rAAV viron, the expression cassette comprises, in 5′ to 3′ order, CMV promoter, the polynucleotide sequence encoding the activated Parkin, R2V17, 3′UTR (globin), and pAGH-Hs.

In particular embodiments of the rAAV viron, e.g., AAV2 rAAV viron or AAV9 rAAV viron, the expression cassette comprises, in 5′ to 3′ order, CMV promoter, the polynucleotide sequence encoding the activated Parkin, and pAGH-Hs.

In particular embodiments of the foregoing rAAV virons, e.g., AAV2 rAAV virons or AAV9 rAAV virons, the order of the elements 5′ to the polynucleotide sequence encoding the activated Parkin are reversed so that the promoter precedes the enhancer elements or the enhancer element precedes the promoter element.

### Pharmaceutical Compositions and Kits

In an aspect, the disclosure provides pharmaceutical compositions comprising the rAAV virion of the disclosure and one or more pharmaceutically acceptable carriers, diluents, or excipients.

For purposes of administration, e.g., by injection, various solutions can be employed, such as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as Pluronic™ F-68 at 0.001% or 0.01%. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include but are not limited to sterile aqueous solutions or dispersions and sterile powders for the extemporaneous preparation of sterile injectable solutions or dispersions. In all cases the form is sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions may be prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

In another aspect, the disclosure comprises a kit comprising an rAAV virion of the disclosure and instructions for use.

### Methods of Use

In an aspect, the disclosure provides a method of increasing Parkin activity in a cell, comprising contacting the cell with an rAAV of the disclosure. In another aspect, the disclosure provides a method of increasing Parkin activity in a subject, comprising administering to the subject an rAAV of the disclosure. In some embodiments, the cell and/or subject is deficient in Parkin activity and/or comprises a loss-of-function mutation in Parkin. The cell may be a neuron, e.g. a dopaminergic neuron. In some embodiments, the cell and/or subject is deficient in PINK1 activity and/or comprises a loss-of-function mutation in PINK1. In various embodiments, the activated Parkin, when expressed in the cell or subject.

In certain embodiments, the cell is contacted with or the subject is administered a rAAV viron, e.g., an AAV2 rAAV viron or an AAV9 rAAV viron, comprising an expression cassette disclosed herein.

In certain embodiments, the cell is contacted with or the subject is administered a rAAV viron, e.g., AAV2 rAAV viron or AAV9 rAAV viron, comprising an expression cassette comprising, in 5′ to 3′ order, HuBA promoter, the polynucleotide sequence encoding the activated Parkin, WPRE(x), and pAGlobin-Oc.

In certain embodiments, the cell is contacted with or the subject is administered a rAAV viron, e.g., AAV2 rAAV viron or AAV9 rAAV viron, comprising an expression cassette comprising, in 5′ to 3′ order, CMV promoter, TPL-eMLP enhancer, the polynucleotide sequence encoding the activated Parkin, WPRE(r), and pAGlobin-Oc.

In certain embodiments, the cell is contacted with or the subject is administered a rAAV viron, e.g., AAV2 rAAV viron or AAV9 rAAV viron, comprising an expression cassette comprising, in 5′ to 3′ order, Syn promoter, the polynucleotide sequence encoding the activated Parkin, WPRE(r), 3′UTR (globin), and pAGH-Bt.

In certain embodiments, the cell is contacted with or the subject is administered a rAAV viron, e.g., AAV2 rAAV viron or AAV9 rAAV viron, comprising an expression cassette comprising, in 5′ to 3′ order, CBA promoter, the polynucleotide sequence encoding the activated Parkin, and pAGH-Bt.

In certain embodiments, the cell is contacted with or the subject is administered a rAAV viron, e.g., AAV2 rAAV viron or AAV9 rAAV viron, comprising an expression cassette comprising, in 5′ to 3′ order, EF1α promoter, the polynucleotide sequence encoding the activated Parkin, and pAGlobin-Oc.

In certain embodiments, the cell is contacted with or the subject is administered a rAAV viron, e.g., AAV2 rAAV viron or AAV9 rAAV viron, comprising an expression cassette comprising, in 5′ to 3′ order, HuBA promoter, the polynucleotide sequence encoding the activated Parkin, R2V17, and pAGH-Bt.

In certain embodiments, the cell is contacted with or the subject is administered a rAAV viron, e.g., AAV2 rAAV viron or AAV9 rAAV viron, comprising an expression cassette comprising, in 5′ to 3′ order, Syn promoter, the polynucleotide sequence encoding the activated Parkin, WPRE(x), 3′UTR (globin), and pAGH-Hs.

In certain embodiments, the cell is contacted with or the subject is administered a rAAV viron, e.g., AAV2 rAAV viron or AAV9 rAAV viron, comprising an expression cassette comprising, in 5′ to 3′ order, CaMKIIa promoter, the polynucleotide sequence encoding the activated Parkin, WPRE(r), and pAGH-Hs.

In certain embodiments, the cell is contacted with or the subject is administered a rAAV viron, e.g., AAV2 rAAV viron or AAV9 rAAV viron, comprising an expression cassette comprising, in 5′ to 3′ order, CMV promoter, TPL-eMLP enhancer, the polynucleotide sequence encoding the activated Parkin, WPRE(r), and pAGH-Hs.

In certain embodiments, the cell is contacted with or the subject is administered a rAAV viron, e.g., AAV2 rAAV viron or AAV9 rAAV viron, comprising an expression cassette comprising, in 5′ to 3′ order, HuBA promoter, the polynucleotide sequence encoding the activated Parkin, and pAGH-Hs.

In certain embodiments, the cell is contacted with or the subject is administered a rAAV viron, e.g., AAV2 rAAV viron or AAV9 rAAV viron, comprising an expression cassette comprising, in 5′ to 3′ order, CMV promoter, TPL/eMLP enhancer, the polynucleotide sequence encoding the activated Parkin, R2V17, 3′UTR (globin), and pAGH-Bt.

In certain embodiments, the cell is contacted with or the subject is administered a rAAV viron, e.g., AAV2 rAAV viron or AAV9 rAAV viron, comprising an expression cassette comprising, in 5′ to 3′ order, EF1α promoter, the polynucleotide sequence encoding the activated Parkin, WPRE(r), and pAGH-Bt.

In certain embodiments, the cell is contacted with or the subject is administered a rAAV viron, e.g., AAV2 rAAV viron or AAV9 rAAV viron, comprising an expression cassette comprising, in 5′ to 3′ order, Syn promoter, the polynucleotide sequence encoding the activated Parkin, R2V17, and pAGlobin-Oc.

In certain embodiments, the cell is contacted with or the subject is administered a rAAV viron, e.g., AAV2 rAAV viron or AAV9 rAAV viron, comprising an expression cassette comprising, in 5′ to 3′ order, CaMKIIa promoter, the polynucleotide sequence encoding the activated Parkin, R2V17, and pAGlobin-Oc.

In certain embodiments, the cell is contacted with or the subject is administered a rAAV viron, e.g., AAV2 rAAV viron or AAV9 rAAV viron, comprising an expression cassette comprising, in 5′ to 3′ order, CBA promoter, the polynucleotide sequence encoding the activated Parkin, WPRE(x), 3′UTR (globin), and pAGH-Hs.

In certain embodiments, the cell is contacted with or the subject is administered a rAAV viron, e.g., AAV2 rAAV viron or AAV9 rAAV viron, comprising an expression cassette comprising, in 5′ to 3′ order, CBA promoter, the polynucleotide sequence encoding the activated Parkin, 3′UTR (globin), and pAGlobin-Oc.

In certain embodiments, the cell is contacted with or the subject is administered a rAAV viron, e.g., AAV2 rAAV viron or AAV9 rAAV viron, comprising an expression cassette comprising, in 5′ to 3′ order, CaMKIIa promoter, the polynucleotide sequence encoding the activated Parkin, R2V17, and pAGH-Bt.

In certain embodiments, the cell is contacted with or the subject is administered a rAAV viron, e.g., AAV2 rAAV viron or AAV9 rAAV viron, comprising an expression cassette comprising, in 5′ to 3′ order, EF1α promoter, the polynucleotide sequence encoding the activated Parkin, R2V17, 3′UTR (globin), and pAGH-Hs.

In certain embodiments, the cell is contacted with or the subject is administered a rAAV viron, e.g., AAV2 rAAV viron or AAV9 rAAV viron, comprising an expression cassette comprising, in 5′ to 3′ order, CMV promoter, the polynucleotide sequence encoding the activated Parkin, R2V17, 3′UTR (globin), and pAGH-Hs.

In certain embodiments, the cell is contacted with or the subject is administered a rAAV viron, e.g., AAV2 rAAV viron or AAV9 rAAV viron, comprising an expression cassette comprising, in 5′ to 3′ order, CMV promoter, the polynucleotide sequence encoding the activated Parkin, and pAGH-Hs.

In particular embodiments of the foregoing rAAV virions, e.g., AAV2 rAAV virons or AAV9 rAAV virons, the order of the elements 5′ to the polynucleotide sequence encoding the activated Parkin are reversed so that the promoter precedes the enhancer elements or the enhancer element precedes the promoter element.

Efficacy of the activated Parkin may be determined as an increase relative to untreated cells/controls or relative to treatment with a reference Parkin protein, in one or more assays, such as, for example and without limitation: (1) expression of the active Parkin protein; (2) increased ubiquitination of mitochondrial proteins; (3) improved mitophagy; (4) reduced cellular toxicity; (5) reduced oxidative stress; and/or (6) increase survival of neurons, e.g., dopaminergic neurons. In particular embodiments, the increase is at least 10%, at least 20%, at least 30%, at least 40%, at least 50%, at least 60%, at least 70%, at least 80%, at least 90%, at least two-fold, as least three-fold, at least four-fold, at least five-fold, at least 10-fold, at least 20-fold, at least 50-fold, or at least 100-fold. The foregoing parameters and others can be measured by methods well known in the art, including but limited to those described in Example 4-5.

In some embodiments, the method promotes survival of neurons in cell culture and/or in vivo. The neuron may be dopaminergic neuron. Survival may be measured using one or more assays, such as those described in the Examples below. In particular embodiments, the survival is increased at least 10%, at least 20%, at least 30%, at least 40%, at least 50%, at least 60%, at least 70%, at least 80%, at least 90%, at least two-fold, as least three-fold, at least four-fold, at least five-fold, at least 10-fold, at least 20-fold, at least 50-fold, or at least 100-fold.

### Methods of Treatment

In another aspect, the disclosure provides a method of treating a disease or disorder in a subject in need thereof, comprising administering to the subject an effective amount of an rAAV virion of the disclosure. In some embodiments, the subject suffers from a genetic deficiency in Parkin expression or function. The subject may suffer from a genetic deficiency (whether diagnosed or not diagnosed) in PRKN (i.e., PARK2, AR-DJ, Ubiquitin E3 Ligase), PARK7 (i.e., DJ-1), PINK1 (i.e., PARK6, PTEN-induced putative kinase 1, BRPK), LRRK2, SNCA (i.e., PARK1, PARK4, alpha-synuclein). In some embodiments, the subject suffers from a genetic deficiency in PINK1 expression or function. In some embodiments, the subject suffers from a genetic deficiency in DJ-1 expression or function.

In certain embodiments, the subject is administered a rAAV viron, e.g., an AAV2 rAAV viron or an AAV9 rAAV viron, comprising an expression cassette disclosed herein.

In certain embodiments, the subject is administered a rAAV viron, e.g., AAV2 rAAV viron or AAV9 rAAV viron, comprising an expression cassette comprising, in 5′ to 3′ order, HuBA promoter, the polynucleotide sequence encoding the activated Parkin, WPRE(x), and pAGlobin-Oc.

In certain embodiments, the subject is administered a rAAV viron, e.g., AAV2 rAAV viron or AAV9 rAAV viron, comprising an expression cassette comprising, in 5′ to 3′ order, CMV promoter, TPL-eMLP enhancer, the polynucleotide sequence encoding the activated Parkin, WPRE(r), and pAGlobin-Oc.

In certain embodiments, the subject is administered a rAAV viron, e.g., AAV2 rAAV viron or AAV9 rAAV viron, comprising an expression cassette comprising, in 5′ to 3′ order, Syn promoter, the polynucleotide sequence encoding the activated Parkin, WPRE(r), 3′UTR (globin), and pAGH-Bt.

In certain embodiments, the subject is administered a rAAV viron, e.g., AAV2 rAAV viron or AAV9 rAAV viron, comprising an expression cassette comprising, in 5′ to 3′ order, CBA promoter, the polynucleotide sequence encoding the activated Parkin, and pAGH-Bt.

In certain embodiments, the subject is administered a rAAV viron, e.g., AAV2 rAAV viron or AAV9 rAAV viron, comprising an expression cassette comprising, in 5′ to 3′ order, EF1α promoter, the polynucleotide sequence encoding the activated Parkin, and pAGlobin-Oc.

In certain embodiments, the subject is administered a rAAV viron, e.g., AAV2 rAAV viron or AAV9 rAAV viron, comprising an expression cassette comprising, in 5′ to 3′ order, HuBA promoter, the polynucleotide sequence encoding the activated Parkin, R2V17, and pAGH-Bt.

In certain embodiments, the subject is administered a rAAV viron, e.g., AAV2 rAAV viron or AAV9 rAAV viron, comprising an expression cassette comprising, in 5′ to 3′ order, Syn promoter, the polynucleotide sequence encoding the activated Parkin, WPRE(x), 3′UTR (globin), and pAGH-Hs.

In certain embodiments, the subject is administered a rAAV viron, e.g., AAV2 rAAV viron or AAV9 rAAV viron, comprising an expression cassette comprising, in 5′ to 3′ order, CaMKIIa promoter, the polynucleotide sequence encoding the activated Parkin, WPRE(r), and pAGH-Hs.

In certain embodiments, the subject is administered a rAAV viron, e.g., AAV2 rAAV viron or AAV9 rAAV viron, comprising an expression cassette comprising, in 5′ to 3′ order, CMV promoter, TPL-eMLP enhancer, the polynucleotide sequence encoding the activated Parkin, WPRE(r), and pAGH-Hs.

In certain embodiments, the subject is administered a rAAV viron, e.g., AAV2 rAAV viron or AAV9 rAAV viron, comprising an expression cassette comprising, in 5′ to 3′ order, HuBA promoter, the polynucleotide sequence encoding the activated Parkin, and pAGH-Hs.

In certain embodiments, the subject is administered a rAAV viron, e.g., AAV2 rAAV viron or AAV9 rAAV viron, comprising an expression cassette comprising, in 5′ to 3′ order, CMV promoter, TPL/eMLP enhancer, the polynucleotide sequence encoding the activated Parkin, R2V17, 3′UTR (globin), and pAGH-Bt.

In certain embodiments, the subject is administered a rAAV viron, e.g., AAV2 rAAV viron or AAV9 rAAV viron, comprising an expression cassette comprising, in 5′ to 3′ order, EF1α promoter, the polynucleotide sequence encoding the activated Parkin, WPRE(r), and pAGH-Bt.

In certain embodiments, the subject is administered a rAAV viron, e.g., AAV2 rAAV viron or AAV9 rAAV viron, comprising an expression cassette comprising, in 5′ to 3′ order, Syn promoter, the polynucleotide sequence encoding the activated Parkin, R2V17, and pAGlobin-Oc.

In certain embodiments, the subject is administered a rAAV viron, e.g., AAV2 rAAV viron or AAV9 rAAV viron, comprising an expression cassette comprising, in 5′ to 3′ order, CaMKIIa promoter, the polynucleotide sequence encoding the activated Parkin, R2V17, and pAGlobin-Oc.

In certain embodiments, the subject is administered a rAAV viron, e.g., AAV2 rAAV viron or AAV9 rAAV viron, comprising an expression cassette comprising, in 5′ to 3′ order, CBA promoter, the polynucleotide sequence encoding the activated Parkin, WPRE(x), 3′UTR (globin), and pAGH-Hs.

In certain embodiments, the subject is administered a rAAV viron, e.g., AAV2 rAAV viron or AAV9 rAAV viron, comprising an expression cassette comprising, in 5′ to 3′ order, CBA promoter, the polynucleotide sequence encoding the activated Parkin, 3′UTR (globin), and pAGlobin-Oc.

In certain embodiments, the subject is administered a rAAV viron, e.g., AAV2 rAAV viron or AAV9 rAAV viron, comprising an expression cassette comprising, in 5′ to 3′ order, CaMKIIa promoter, the polynucleotide sequence encoding the activated Parkin, R2V17, and pAGH-Bt.

In certain embodiments, the subject is administered a rAAV viron, e.g., AAV2 rAAV viron or AAV9 rAAV viron, comprising an expression cassette comprising, in 5′ to 3′ order, EF1α promoter, the polynucleotide sequence encoding the activated Parkin, R2V17, 3′UTR (globin), and pAGH-Hs.

In certain embodiments, the subject is administered a rAAV viron, e.g., AAV2 rAAV viron or AAV9 rAAV viron, comprising an expression cassette comprising, in 5′ to 3′ order, CMV promoter, the polynucleotide sequence encoding the activated Parkin, R2V17, 3′UTR (globin), and pAGH-Hs.

In certain embodiments, the subject is administered a rAAV viron, e.g., AAV2 rAAV viron or AAV9 rAAV viron, comprising an expression cassette comprising, in 5′ to 3′ order, CMV promoter, the polynucleotide sequence encoding the activated Parkin, and pAGH-Hs.

In particular embodiments of the foregoing rAAV virons, e.g., AAV2 rAAV virons or AAV9 rAAV virons, the order of the elements 5′ to the polynucleotide sequence encoding the activated Parkin are reversed so that the promoter precedes the enhancer elements or the enhancer element precedes the promoter element.

In some embodiments, the disease or disorder is Parkinson’s disease. The disclosure provides treatments for any of various neurodegenerative diseases. For example, the rAAV virions of the disclosure treat Early Onset Parkinson’s Disease (EOPD) or Juvenile PD, which are also known as young onset, early onset, juvenile onset, and autosomal recessive early onset Parkinson’s disease.

The rAAV virions of the disclosure further treat idiopathic PD, nigrostriatal degeneration, dopamine insufficiency due to primary dopamine neuron loss, sporadic PD, PD etiology unknown, neurodegenerative disease associated with loss of function and/or frank neuronal degeneration of dopaminergic neurons in the midbrain (including the substantia nigra and/or ventral tegmental area) with unknown etiology or idiopathic, and sporadic onset neurodegenerative disease.

The methods of the disclosure may prevent loss of dopaminergic neurons in the substantia nigra in various disorders, including, without limitation, those associated with aging and/or genetic causes and/or Parkinson’s disease with unknown etiology (i.e., idiopathic PD). Various neurodegenerative conditions associated with primary loss of neurons in the substantia nigra with unknown etiology or known etiology may be treated.

In some embodiments, the compositions of the disclosure may act as therapeutics with neuroprotective and neurorestorative potential to halt and/or prevent further loss of dopaminergic neurons in the substantia nigra due to absence of, or mutations in the PARK2 or PINK1 gene. The compositions of the disclosure may be administered as neuroprotection therapy to mitigate nigrostriatal neurodegeneration, loss of dopaminergic neurons located in the substantia nigra region of the midbrain, in patients with early onset Parkinson’s disease as a consequence of mutations or deletions in the PARK 2 and/or PINK1 gene.

The AAV-mediated delivery of activated Parkin protein to the CNS may improve anatomical, neurochemical, and behavioral measures indicative of neuroprotection and/or neurorestoration of dopaminergic nigrostriatal system.

Combination therapies are also contemplated by the invention. Combination therapy may comprise administration of an rAAV virion of the disclosure and either or both of 1-3,4-dihydroxyphenylalanine (L-DOPA) and dopamine agonists. In some embodiments, administration of the rAAV virion decreases the need to administer L-DOPA and/or DA. Combination as used herein includes simultaneous treatment or sequential treatment. Combinations of methods of the invention with standard medical treatments (e.g., corticosteroids or topical pressure reducing medications) are specifically contemplated, as are combinations with novel therapies. In some cases, a subject may be treated with a steroid to prevent or to reduce an immune response to administration of a rAAV described herein.

A therapeutically effective amount of the rAAV vector, e.g. for intravenous injection, is a dose of rAAV ranging from about 1e7 vg/kg to about 5e15 vg/kg, or about 1e7 vg/kg to about 1e14 vg/kg, or about 1e8 vg/kg to about 1e14 vg/kg, or about 1e9 vg/kg to about 1e13 vg/kg, or about 1e9 vg/kg to about 1e12 vg/kg, or about 1e7 vg/kg to about 5e7 vg/kg, or about 1e8 vg/kg to about 5e8 vg/kg, or about 1e9 vg/kg to about 5e9 vg/kg, or about 1e10 vg/kg to about 5e10 vg/kg, or about 1e11 vg/kg to about 5e11 vg/kg, or about 1e12 vg/kg to about 5e12 vg/kg, or about 1e13 vg/kg to about 5e13 vg/kg, or about 1e14 vg/kg to about 5e14 vg/kg, or about 1e15 vg/kg to about 5e15 vg/kg. The invention also comprises compositions comprising these ranges of rAAV vector.

For example, in particular embodiments, a therapeutically effective amount of rAAV vector is a dose of about 1e10 vg/kg, about 2e10 vg/kg, about 3e10 vg/kg, about 4e10 vg/kg, about 5e10 vg/kg, about 6e10 vg/kg, about 7e10 vg/kg, about 8e10 vg/kg, about 9e10 vg/kg, about 1e12 vg/kg, about 2e12 vg/kg, about 3e12 vg/kg, about 4e12 vg/kg and 5e12 vg/kg. The invention also comprises compositions comprising these doses of rAAV vector.

In some embodiments, for example where direct injection into substantia nigra is performed, a therapeutically effective amount of rAAV vector is a dose in the range of 1e7/hemisphere vg to 1e11 vg/hemisphere, or about 1e7 vg/hemisphere, about 1e8 vg/hemisphere, about 1e9 vg/hemisphere, about 1e10 vg/hemisphere, or about 1e11 vg/hemisphere.

In some embodiments, for example where direct injection into the putamen (intraputaminal) is performed, a therapeutically effective amount of rAAV vector is a dose in the range of 1e9 vg/hemisphere to 6e11 vg/hemisphere, or about 1e9 vg/hemisphere, about 1e10 vg/hemisphere, about 1e11 vg, about 2e11 vg/hemisphere, or about 3e11 vg/hemisphere, or about 6e11 vg/hemisphere .

In some cases, the therapeutic composition comprises more than about 1e9, 1e10, or 1e11 genomes of the rAAV vector per volume of therapeutic composition injected. In some cases, the therapeutic composition comprises more than about 1e9, 1e10, or 1e11 genomes of the rAAV vector per volume of therapeutic composition injected. In some cases, the therapeutic composition comprises more than approximately 1e10, 1e11, 1e12, or 1e13 genomes of the rAAV vector per mL. In certain embodiments, the therapeutic composition comprises less than about 1e14, 1e13 or lele12 genomes of the rAAV vector per mL.

In some embodiments, the disclosure provides a method of treating and/or preventing Parkinson’s disease, comprising administering a vector of the disclosure, optionally before, during or after the onset of disease. The Parkinson’s disease may be early onset Parkinson’s disease (EOPD). In some embodiments, the method alleviates one or more symptoms of Parkinson’s disease, e.g. EOPD. It may reduce motor complications associated with neurodegeneration, nigrostriatal degeneration, and/or ataxia; reduce the need for antiparkinsonian pharmacotherapy (including but not limited to L-DOPA and dopaminergic agonists); restore the function of degenerating neurons; and/or protect neurons from degeneration.

Evidence of functional improvement, clinical benefit or efficacy in patients may be assessed by the analysis of surrogate markers of enhanced nigrostriatal function such as [18F]fluoro-L-dopa positron emission tomography (PET) uptake in the putamen and midbrain region of the substantia nigra, or markers of presynaptic dopamine terminal activity such as the dopamine transporter (DaT) via DaT-SPECT imaging of putamen. Evidence of symptomatic, clinical benefit may be determined using standard Parkinson’s disease rating scales, such as the Unified Parkinson’s Disease Rating Scale (UPDRS) or the Movement Disorder Society-sponsored version of the UPDRS (MDS-UPDRS), evaluated with and without concomitant anti-parkinsonian medications. These or similar scales, as well as patient-reported outcomes on quality of life, may demonstrate improvements in both motor and non-motor components of the disease. Further methods of assessing treatment effects are known in the art. These include but are not limited to the methods used in Examples 6.

### Administration of Compositions

Administration of an effective dose of the compositions may be by routes standard in the art including, but not limited to, systemic, local, direct injection, intravenous, cerebral, cerebrospinal, intrathecal, intracisternal, intraputaminal, intrahippocampal, intra-striatal (putamen and/or caudate), or intra-cerebroventricular administration. In some cases, administration comprises intravenous, cerebral, cerebrospinal, intrathecal, intracisternal, intraputaminal, intrahippocampal, intra-striatal (putamen and/or caudate), or intra-cerebroventricular injection. Administration may be performed by intrathecal injection with or without Trendelenberg tilting.

In some embodiments, the disclosure provides for local administration and systemic administration of an effective dose of rAAV and compositions of the invention. For example, systemic administration may be administration into the circulatory system so that the entire body is affected. Systemic administration includes parental administration through injection, infusion or implantation.

In particular, administration of rAAV of the present invention may be accomplished by using any physical method that will transport the rAAV recombinant vector into the target tissue of an animal. Administration includes, but is not limited to, injection into the central nervous system (CNS) or cerebrospinal fluid (CSF) and/or directly into the brain.

In some embodiments, the methods of the disclosure comprise direct intraparenchymal delivery, e.g., to the region of the midbrain (or directly above the midbrain), including the region of the substantia nigra (and surrounding regions) by neurosurgical procedure. Infusion may be performed using specialized cannula, catheter, syringe/needle using an infusion pump. Optionally, targeting of the injection site may be accomplished with MRI-guided imaging. Administration may comprise delivery of an effective amount of the rAAV virion, or a pharmaceutical composition comprising the rAAV virion, to the CNS. These may be achieved, e.g., via intracisternal magna infusion with Trendelenburg tilting procedure, or intracisternal magna infusion without Trendelenburg tilting procedure, intrathecal infusion with Trendelenburg tilting procedure, or intrathecal infusion without Trendelenburg tilting procedure. The compositions of the disclosure may further be administered intravenously.

Direct delivery to the CNS could involve targeting specific neuronal regions or more general brain regions containing neuronal targets. Individual patient brain region and/or neuronal target(s) selection and subsequent intraoperative delivery of AAV could by accomplished using a number of imaging techniques (MRI, CT, CT combined with MRI merging) and employing any number of software planning programs (e.g., Stealth System, Clearpoint Neuronavigation System, Brainlab, Neuroinspire etc). Brain region targeting and delivery could involve us of standard stereotactic frames (Leksell, CRW) or using frameless approaches with or without intraoperative MRI. Actual delivery of AAV may be by injection through needle or cannulae with or without inner lumen lined with material to prevent adsorption of AAV vector (e.g. Smartflow cannulae, MRI Interventions cannulae). Delivery device interfaces with syringes and automated infusion or microinfusion pumps with preprogrammed infusion rates and volumes. The syringe/needle combination or just the needle may be interfaced directly with the stereotactic frame. Infusion may include constant flow rate or varying rates with convection enhanced delivery.

### EXAMPLES

### Example 1: Bioactivity in Vitro

Plasmid vectors having an AAV expression cassette encoding each of the following Parkin mutants and are constructed using conventional cloning methods:

| | | | |
|---|---|---|---|
| N273K + W403A + F146A | N273K + W403A + F146A + Y143A | N273K + W403A + F146A + Ser131A | N273K + W403A + F146A + Y143A+ Ser131A |
| N273K + W403A + C457S | N273K + W403A + C457S + Y143A | N273K + W403A + C457S + Ser131A | N273K + W403A + C457S + Y143A+ Ser131A |
| N273K + W403A + F463A | N273K + W403A + F463A + Y143A | N273K + W403A + F463A + Ser131A | N273K + W403A + F463A + Y143A+ Ser131A |
| N273K + W403A + F146A + C457S | N273K + W403A + F146A + C457S + Y143A | N273K + W403A + F146A + C457S + Ser131A | N273K + W403A + F146A + C457S + Y143A+ Ser131A |
| N273K + W403A + F146A + C457S + F463A | N273K + W403A + F146A + C457S + F463A + Y143A | N273K + W403A + F146A + C457S + F463A + Ser131A | N273K + W403A + F146A + C457S + F463A + Y143A+ Ser131A |
| W403A + F146A | W403A + F146A + Y143A | W403A + F146A + Ser131A | W403A + F146A + Y143A+ Ser131A |
| W403A + C457S | W403A + C457S + Y143A | W403A + C457S + Ser131A | W403A + C457S + Y143A+ Ser131A |
| W403A + F463A | W403A + F463A + Y143A | W403A + F463A + Ser131A | W403A + F463A + Y143A+ Ser131A |
| W403A + F146A + C457S | W403A + F146A + C457S + Y143A | W403A + F146A + C457S + Ser131A | W403A + F146A + C457S + Y143A+ Ser131A |
| W403A + F146A + C457S + F463A | W403A + F146A + C457S + F463A + Y143A | W403A + F146A + C457S + F463A + Ser131A | W403A + F146A + C457S + F463A + Y143A+ Ser131A |

MTS-TMD of PINK1[1-110], fused to ΔParkin[76-465]
MTS-TMD of PINK1[1-110], fused to ΔParkin[141-465]
MTS-TMD of PINK1[1-110], fused to ΔParkin[76-465]F146A+W403A
MTS-TMD of PINK1[1-110], fused to ΔParkin[141-465]F146A+W403A
MTS-TMD of PINK1 [1-110], fused to ΔParkin[76-465]W403A + F463A
MTS-TMD of PINK1[1-110], fused to ΔParkin[141-465]W403A + F463A
MTS-TMD of PINK1 [1-110], fused to ΔParkin[76-465]W403A + C457S
MTS-TMD of PINK1[1-110], fused to ΔParkin[141-465]W403A + C457S
MTS-TMD of PINK1[1-110] F104M, fused to ΔParkin[76-465]
MTS-TMD of PINK1[1-110] F104M, fused to ΔParkin[141-465]
MTS-TMD of PINK1[1-110] F104M, fused to ΔParkin[76-465]F146A+W403A
MTS-TMD of PINK1[1-110] F104M, fused to ΔParkin[141-465]F146A+W403A
MTS-TMD of PINK1[1-110] F104M, fused to ΔParkin[76-465]W403A + F463A
MTS-TMD of PINK1[1-110] F104M, fused to ΔParkin[141-465]W403A + F463A
MTS-TMD of PINK1[1-110] F104M, fused to ΔParkin[76-465]W403A + C457S
MTS-TMD of PINK1[1-110] F104M, fused to ΔParkin[141-465]W403A + C457S

Constructs are screened for expression of Parkin by Western Blot, ELISA and/or immunolabeling following in vitro transfection of HEK293, HeLa cells, transduction of rat primary neurons, and/or ChoLec2 cells.

Selected constructs showing Parkin expression are transfected into, or converted to AAV virions using a helper-free packaging system and used to transduce, ChoLec2 and/or SH-SY5Y cells. Cells are treated with uncoupling agents (carbonyl cyanide 3-chlorophenylhydrazone [CCCP] or carbonyl cyanide 4-(trifluoromethoxy)phenylhydrazone FCCP]), an assay for mitochondrial damage. Fluorescence microscopy is used to measure localization of the Parkin mutants to mitochondria. Cells are also tested for clearance of damaged mitochondria by measuring colocalization of exogenous Parkin and Translocase of the outer mitochondrial membrane complex subunit 20 (TOMM20) and by Western blot of the mitochondrial membrane fraction. Levels of markers of autophagosomes (e.g., LC3) are also measured.

Parkin mutants are further assayed to for their ability to enhance cell survival and to normalize mitochondrial morphology and function, such as mitigation of reactive oxygen species is assessed by MitoSOX assay.

To further demonstrate bioactivity of Parkin constructs, modifications of Parkin substrates is measured, e.g., ubiquitination or the total expression levels AIMP2, CISD1, Miro, STEP-61, RTP-801, Porin, Mitofusin, PARIS, PGC-1α, compared to appropriate controls (endogenous proteins, e.g., β-actin).

Selected AAV virions are further assessed in primary neurons from rodents lacking normal PARK2 or PARK6 gene and in human, patient-derived cells lacking normal PARK2 or PARK6 gene. The neurons may be differentiated into dopaminergic neurons before, during, or after being contacted with the AAV virions. The bioactivity assays described above are repeated in the primary neuron or patient-derived cell assays.

### Example 2: In Vivo Efficacy

Treatment with AAV virion encoding selected Parkin constructs is tested in animal models of disease. Specifically mouse, rat, or non-human primate (NHP) are treated with dopaminergic neurotoxin to induce neurological disease. Neurotoxin used in the experiments include 1-methyl-4-phenyl-1,2,3,6-tetrahydropyridine (MPTP) and 6-hydroxydopamine.

Treatment with AAV virion encoding selected Parkin constructs is also tested in mouse or rat models having loss of function (e.g. null) mutations in the PARK2 or PARK6 gene. Neuroprotective and neurorestorative effects of treatment are measured.

Evaluation includes testing for prevention of loss, or rescue from further degeneration, of dopaminergic neurons in the substantia nigra and/or ventral tegmental area. Neuroprotective/neurorestorative effects on nigrostriatal system are measured using techniques disclosed in, e.g., Kirik et al., Eur J Neurosci, 2000, such as quantification of the number of neuronal cell bodies, general morphology (e.g., size, shape) of neuron cell bodies and their axonal processes, and the integrity of their axonal projections in route to other brain regions (e.g., striatum). Characterization of dopaminergic neurons (quantitation of neuron number) and fiber density (optical densitometry) is accomplished using immunolabeling for tyrosine hydroxylase and/or vesicular monoamine transporter. Neurochemical levels of dopamine and/or its metabolites (e.g., 3,4-dihydroxyphenylacetic acid [DOPAC], homovanillic acid [HVA]) in striatum and/or substantia nigra are also quantified.

Characterization of the functional/behavioral consequences of treatment with AAV virions encoding Parkin constructs is accomplished by examining motor behaviors using known testing paradigms to characterize nigrostriatal function in the rodent (Bjorklund et al. Br Res, 2000; Kirik et al., Nat Neurosci, 2004), including but not limited to amphetamine-induced rotation, spontaneous rotation, forelimb use preference, cylinder test, adjusted stepping task, general locomotor behavior in open field, and rotorod. NHPs are evaluated by behavioral testing using a NHP equivalent of the Unified Parkinson’s Disease Rating Scale (Kordower et al., Ann Neurol, 2006).

### Example 4: Prevention of Neuronal Loss: 6-ohda Model of Parkinson’s Disease

Parkin variants were tested in an assay known in the art as a model for the neuronal damage caused by Parkinson’s disease, a 6-OHDA toxicity model as described, for example, in Simola et al. Neurotox Res. 2007 Apr;11(3-4): 151-67 (2007); Hanrott et al. J. Biol. Chem. 281:5373-82 (2006). The model produces robust dopaminergic neuron oxidative stress and neuron loss, hallmarks of the disease pathology in Parkinson’s patients.

Table 8 summarizes the specific AAV constructs evaluated in these experiments.

TABLE 8

Constructs Tested

| Abbreviation | Modifications to wild-type Parkin | FIG. | Vector Genome SEQ ID NO: | Protein SEQ ID NO: |
|---|---|---|---|---|
| CON GFP | Control = Green Fluorescent Protein | None | None | None |
| WT | None (wild-type) | None | None | 1 |
| ACT | N273K + W403A + F463A | 14 | 92 | 13 or 93 |
| DEL | Deletion residues 1-141, ubiquitin-like (Ubl) domain + W403A + F463A | 15 | 94 | 95 (+N-terminal methionine) |
| SUP1 | MTS/TM + W403A + F463A | 16 | 96 | 97 |
| SUP2 | MTS/TM + F104M + W403A + F463A | 17 | 98 | 99 |
| C431F | C431F | 9 | 91 | 90 |

C431F is described in the literature as catalytic center mutation. Fiesel et al. Hum Mutat. 36:774-786 (2015). It was intended as a negative control for Parkin activation.

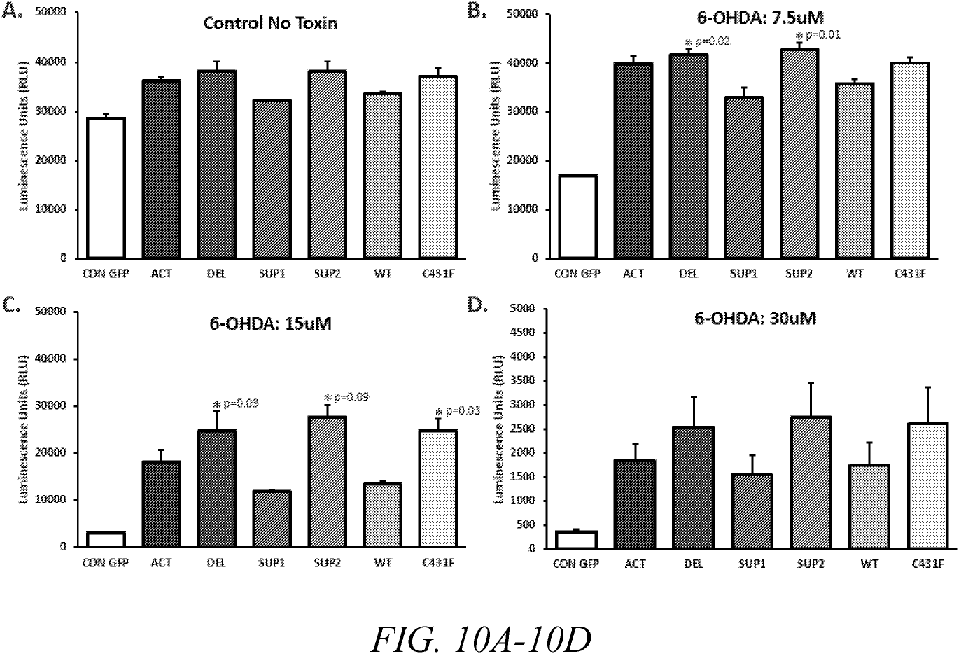

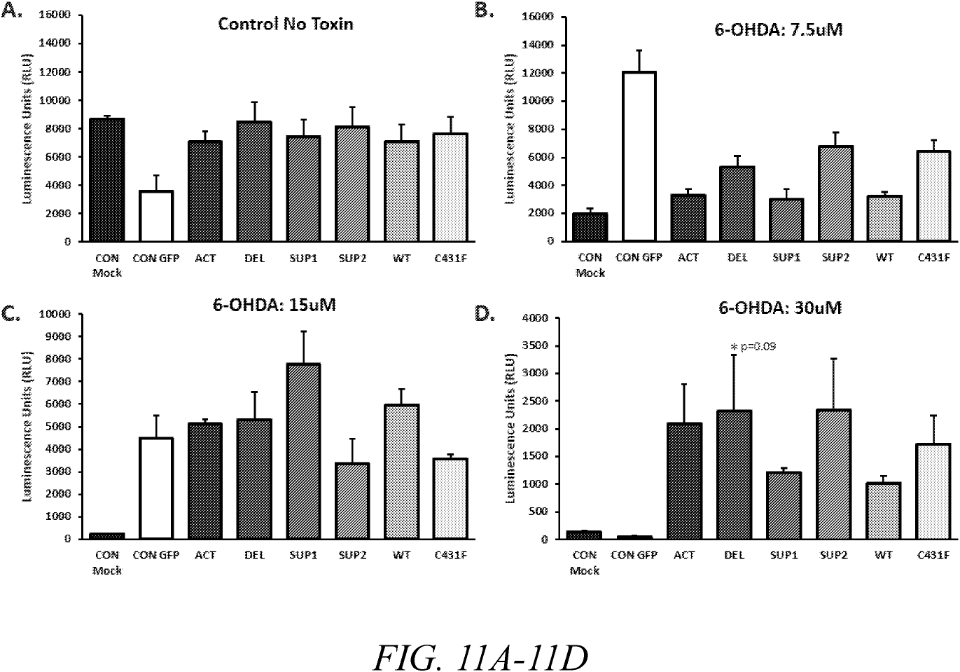

Mitigation of neurotoxic effects of the dopaminergic (DA) toxin 6-OHDA (6-Hydroxydopamine) was evaluated in a rat dopaminergic neuronal cell line (N27-A; END Millipore, Temecula, CA). Cells were seeded in 96 well plates, transfected with plasmid DNA encoding each of the engineered Parkin variants, a fluorescent reporter control, or a mock transfection. After culture for 24 hrs, cells were exposed to 6-OHDA (6-Hydroxydopamine hydrobromide, Sigma-Aldrich, cat. 162957) at concentrations of 7.5 µM, 15 µM, or 30 µM. Cell viability of total neurons in each condition was measured with a luminescence-based assays at three-days (FIGS. 10A-10D; RealTime-GloTM MT Cell Viability Assay; Promega cat. G9712) or nine-days (FIGS. 11A-11D; Cell Titer-Glo 2.0 Assay; Promega cat. G9241) following addition of 6-OHDA. Non-parametric analyses (Kruskal-Wallis) were performed for evaluation of overall effect of transfection condition, and Dunn’s multiple comparison post-hoc analyses differences were performed when appropriate.

In both experiments, the ‘Activated’ and ‘Super’ Parkin constructs prevented toxin dose-dependent decreases in neuronal cell numbers compared to control (“CON GFP”) experiments. Surprisingly, C431F Parkin was more, not less, effective than wild-type Parkin (WT). Furthermore, in both these 6-OHDA experiments, “Activated” Parkin, ΔParkin and “Super Parkin V2” were each superior to wild-type Parkin.

In short, the engineered Parkin constructs tested in this Example are superior to wild-type Parkin in preventing neuronal cell damage in an accepted in vitro model of Parkinson’s disease.

### Example 4: Increase Cell Number and Preserved Mitochondrial Membrane Potential In Human IPSC-Derived PARK2^{-/-} Dopaminergic Neurons

Another accepted in vitro model for Parkinson’s disease is an assay for the prevention of the adverse cellular effects of promoters of oxidative stress in dopaminergic neurons. This includes prevention of the dissipation of the mitochondrial membrane potential in hydrogen peroxide (H₂O₂)-treated Parkin null (i.e., PARK2^{-/-}) dopaminergic neurons. (Ferrari et al. J. Neuroscience Methods 340:108741 (2020); Avazzadeh et al. Brain Sci. 11:373 (2021)) This model uses human cells. Therapeutic approaches that can mitigate loss of dopaminergic neurons in model systems are considered predictive of therapeutic efficacy in Parkinson’s disease, because degeneration of the substantia nigra is observed in subjects having Parkinson’s disease.

iPS-derived human PARK2 -/- dopaminergic neurons (Applied StemCell™, Milpitas, CA) were seeded in 384 well plates and cultured for seven days, then transfected with plasmid DNA encoding each Parkin variant using Viafect (Promega® #E4981). Hydrogen peroxide (150 µM H₂O₂) was added to cells starting at 10 days in culture with 0.1% DMSO as a control (6 wells/condition). Cells were treated with H₂O₂ for 24 and 48 hours prior to evaluation of mitochondrial membrane potential using a red-fluorescent dye that stains mitochondria in live cells and its accumulation is dependent upon membrane potential (MitoTracker™, ThermoFisher® Cat. M7512). Immediately prior to fixation, cells were stained with 250 nM MitoTracker ™ Red CMXRos for 30 minutes according to the standard kit protocol. After fixation, permeabilization, and blocking, cells were subsequently stained with Hoechst nuclear dye. Quantification was achieved by imaging of plates at 20x magnification capturing data from 9 fields/well on a ThermoFisher® CX7LED high content microscope. Automated quantitative image analyses were performed using the ThermoFisher® CellInsight™ software package.

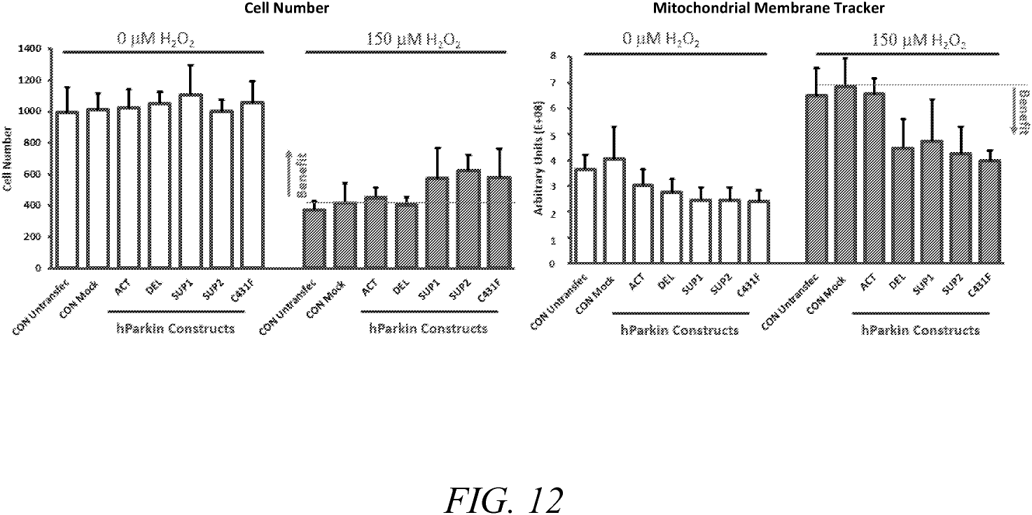

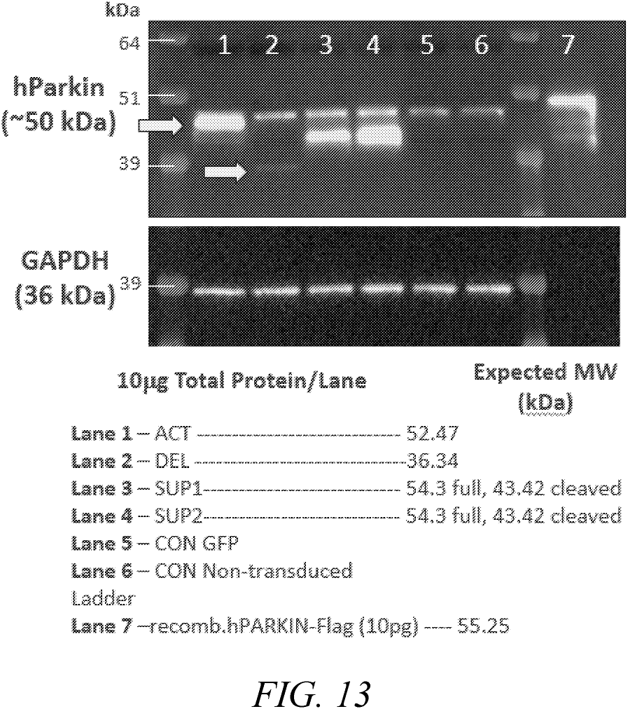

Cell number was markedly reduced following exposure to 150 µM H₂O₂ (FIG. 12, left) in Control Untransfected and Control Mock (CON Mock) conditions. Super Parkin, Super Parkin V2, and Mutation C431F constructs were found to prevent H₂O₂-mediated loss in neuron number (FIG. 12, left) and to prevent dissipation of mitochondrial membrane potential (FIG. 12, right).

The ΔParkin, Super Parkin, Super Parkin V2, and C431F Parkin constructs increased cell numbers observed after H₂O₂ treatment. The Super Parkin, Super Parkin V2, and C431F Parkin constructs prevented an increase in Mitochondrial Membrane Tracker.

### Example 5: Expression of Engineered Parkin Variants From AAV Vector

This Example demonstrates expression of the engineered Parkin constructs from an adeno-associated virus (AAV) vector in a physiologically relevant primary cell— specifically primary cortical neurons.

Primary cortical neuron cultures were prepared using embryonic fetuses (~E18) from pregnant Wistar rat as described by (Banker and Goslin, 1998). Briefly, after isolating brains, hippocampi and cortices were dissected out, washed with Hanks’ balanced salt solution (HBSS), and incubated with trypsin (0.25%) for 15 minutes. Cells were then dissociated by pipetting very gently, with a fine glass pipette, several times. Isolated cells were then plated in neuronal plating medium [Neurobasal medium (Gibco™) containing B27(2%), GlutaMax™ (2 nM) Penn/strep (1%) and Glucose (6.5%), v/v] on poly-L-lysine treated tissue culture plates i at an approximate density of 0.5x10⁶ cells/well of a 6-well dish. Cells were grown in a humidified incubator (37° C., 5% CO2) for approximately three weeks with approximately 1 ml of medium replenished every week. Day 14 neurons were used for AAV transduction.

AAV9 vectors for each of the engineered Parkin constructs were used to transduce the primary neuron cultures at a multiplicity of infection (MOI) of 3 × 10.

Cell lysates were collected after 7 days. Total protein was measured using a BCA kit (Thermo® cat# 23225) and 10 mg of protein was loaded in a 4-12% Bis-Tris gel. Proteins were transferred to a PVDF membrane, blocked in TBS-T + 10% dry milk and incubated overnight with anti-Parkin (CST #2132) antibody. Anti-GAPDH (Abcam ab8245) was also used in experiments as a loading control.

Analyses of Parkin protein expression by Western blot revealed robust protein expression following transduction of primary neurons. Lanes 5 and 6 show the endogenous levels of WT Parkin detected in neurons. Lane 1 revealed Activated Parkin-mediated overexpression of the full-length human Parkin protein with ~52 kDa size. The upper band in Lane 2 represents the endogenous level of human Parkin while the lower band (~36 kDa; arrow) reflects the ΔParkin form of the protein. Lanes 3 and 4 demonstrate Super Parkin-mediated overexpression of human Parkin both full-length (~54 kDa) and its cleaved form (~43 kDa). The cleaved band for the Super Parkin V2 vector is stronger than the cleaved band for the Super Parkin V1, consistent with V2 being more resistant to ubiquitination and subsequent degradation.

### Example 6: In Vivo Testing in MPTP Mouse Model of Nigrostriatal Neurodegeneration

This Example demonstrates treatment of Parkinson’s disease by adeno-associated viral (AAV) vectors expressing the engineered Parkin variants disclosed herein.

The animal model used is the 1-methyl-4-phenyl-1,2,3,6-tetrahydropyridine (MPTP) mouse model of nigrostriatal degeneration. In this model, repeated intraperitoneal injections of the neurotoxin MPTP produces bilateral loss of the dopamine-producing neurons within in the substantia nigra and concomitant depletion of dopamine levels in the striatum.

Unilateral injection of AAV9 vectors encoding each of the four Parkin variants into substantia nigra are performed four weeks prior to MPTP administration. As a positive pharmacological control, a group of mice receive chronic nilotinib injections, a tyrosine kinase inhibitor with some neuroprotective properties, prior to MPTP administration. A summary of the treatment groups and general study design can be found in Table 9.

TABLE 9

| Group | Purpose | n |
|---|---|---|
| FB/Sal | Negative Control | 10 |
| FB/MPTP | MPTP Control | 10 |
| Nilotinib/MPTP | Positive Control | 10 |
| ACT/MPTP | Experimental | 10 |
| DEL/MPTP | Experimental | 10 |
| SUP1/MPTP | Experimental | 10 |
| SUP2/MPTP | Experimental | 10 |

FB=Formulation Buffer Control; Sal=Saline; ACT=Activated Parkin; DEL=ΔParkin;
SUP1=Super Parkin; SUP2=Super Parkin V2; MPTP=1-methyl-4-phenyl-1,2,3,6-tetrahydropyridine

Six days following the first MPTP injection, fresh brain samples are collected. Neurochemical analysis include quantifying levels of dopamine and its metabolites within the striatum using high-performance liquid chromatography (HPLC). Anatomical analyses include quantitation of the number of tyrosine hydroxylase (TH) positive cells within the substantia nigra (SN) (pars compacta; SNc). These data may provide evidence for the potential treatment of loss of dopaminergic neurons in Parkinson’s disease using the engineered Parkin variants disclosed herein.

1. A polynucleotide, comprising a polynucleotide sequence encoding a fusion protein comprising a mitochondrial targeting sequence (MTS); a transmembrane domain (TMD); and a Parkin protein or functional variant or fragment thereof.

2. The polynucleotide of claim 1, wherein the MTS is the MTS of PINK 1 or a functional variant thereof.

3. The polynucleotide of claim 1 or claim 2, wherein the MTS comprises a mitochondrial processing peptidase (MPP) cleavage site.

4. The polynucleotide of any one of claim 1-3, wherein the MTS comprises a polypeptide sequence at least 95% identical to resides 1-34 of human PINK1:
1 MAVRQALGRG LQLGRALLLR FTGKPGRAYG LGRP (SEQ ID NO:66).

5. The polynucleotide of claim 4, wherein the MTS comprises a polypeptide sequence at least 95% identical to residues 1-94 of human PINK1:
1 MAVRQALGRG LQLGRALLLR FTGKPGRAYG LGRPGPAAGC
41 VRGERPGWAA GPGAEPRRVG LGLPNRLRFF RQSVAGLAAR
81 LQRQFVVRAW GCAG (SEQ ID NO: 65).

6. The polynucleotide of claim 5, wherein the MTS comprises a polypeptide sequence identical to residues 1-94 of human PINK1:
1 MAVRQALGRG LQLGRALLLR FTGKPGRAYG LGRPGPAAGC
41 VRGERPGWAA GPGAEPRRVG LGLPNRLRFF RQSVAGLAAR
81 LQRQFVVRAW GCAG (SEQ ID NO: 65).

7. The polynucleotide of any one of claims 1-6, wherein the TMD is the TMD of PINK1 or a functional variant thereof.

8. The polynucleotide of any one of claims 1-7, wherein the TMD comprises a PARL cleavage site.

9. The polynucleotide of any one of claims 1-8, wherein the TMD comprises a polypeptide sequence at least 95% identical to residues 95-110 of human PINK1:
81 PCGRAV FLAFGLGLGL (SEQ ID NO: 67).

10. The polynucleotide of claim 9, wherein the TMD comprises a polypeptide sequence identical to residues 95-110 of human PINK1:
81 PCGRAV FLAFGLGLGL (SEQ ID NO: 67).

11. The polynucleotide of claim 9, wherein the TMD comprises a polypeptide sequence identical to residues 95-110 of human PINK1:
81 PCGRAV FLAMGLGLGL (SEQ ID NO: 68).

12. The polynucleotide of any one of claims 1-11, wherein the fusion protein comprises an MTS-TMD fragment of PINK 1 or a functional variant thereof.

13. The polynucleotide of claim 12, wherein the MTS-TMD fragment comprises a polypeptide sequence at least 95% identical to residues 1-110 of human PINK1:
1 MAVRQALGRG LQLGRALLLR FTGKPGRAYG LGRPGPAAGC
41 VRGERPGWAA GPGAEPRRVG LGLPNRLRFF RQSVAGLAAR
81 LQRQFVVRAW GCAGPCGRAV FLAFGLGLGL (SEQ ID NO: 70).

14. The polynucleotide of claim 12, wherein the MTS-TMD fragment comprises a polypeptide sequence identical to residues 1-110 of human PINK1:
1 MAVRQALGRG LQLGRALLLR FTGKPGRAYG LGRPGPAAGC
41 VRGERPGWAA GPGAEPRRVG LGLPNRLRFF RQSVAGLAAR
81 LQRQFVVRAW GCAGPCGRAV FLAFGLGLGL (SEQ ID NO: 70).

15. The polynucleotide of any one of claims 1-14, wherein the functional variant or fragment thereof is a ΔParkin protein comprising a deletion of the N-terminal ubiquitin-like (Ubl) domain and optionally a deletion of the Ubl-RINGO interdomain linker sequence.

16. The polynucleotide of claim 15, wherein the ΔParkin protein comprises a polypeptide sequence at least 95% identical to residues 141-465 of human Parkin F146A+W403A:
121 SIYNSAYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARAEAASKET IKKTTKPCPR CHVPVEKNGG CMHMKCPQPQ
441 CRLEWCWNCG CEWNRVCMGD HWFDV (SEQ ID NO: 73).

17. The polynucleotide of claim 15, wherein the ΔParkin protein comprises a polypeptide sequence identical to residues 141-465 of human Parkin F146A+W403A:
121 SIYNSAYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARAEAASKET IKKTTKPCPR CHVPVEKNGG CMHMKCPQPQ
441 CRLEWCWNCG CEWNRVCMGD HWFDV (SEQ ID NO: 73).

18. The polynucleotide of claim 15, wherein the ΔParkin protein comprises a polypeptide sequence at least 95% identical to residues 76-465 of human Parkin F146A+W403A:
41 KGQEM
81 NATGGDDPRN AAGGCEREPQ SLTRVDLSSS VLPGDSVGLA
121 VILHTDSRKD SPPAGSPAGR SIYNSAYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARAEAASKET IKKTTKPCPR CHVPVEKNGG CMHMKCPQPQ
441 CRLEWCWNCG CEWNRVCMGD HWFDV (SEQ ID NO: 74).

19. The polynucleotide of claim 15, wherein the ΔParkin protein comprises a polypeptide sequence identical to residues 76-465 of human Parkin F146A+W403A:
41 KGQEM
81 NATGGDDPRN AAGGCEREPQ SLTRVDLSSS VLPGDSVGLA
121 VILHTDSRKD SPPAGSPAGR SIYNSAYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARAEAASKET IKKTTKPCPR CHVPVEKNGG CMHMKCPQPQ
441 CRLEWCWNCG CEWNRVCMGD HWFDV (SEQ ID NO:74).

20. The polynucleotide of any one of claims 1-19 wherein the fusion protein comprises a polypeptide sequence at least 95% identical to the sequence:
1 MAVRQALGRG LQLGRALLLR FTGKPGRAYG LGRPGPAAGC
41 VRGERPGWAA GPGAEPRRVG LGLPNRLRFF RQSVAGLAAR
81 LQRQFVVRAW GCAGPCGRAV FLAFGLGLGL KGQEMNATGG
121 DDPRNAAGGC EREPQSLTRV DLSSSVLPGD SVGLAVILHT
161 DSRKDSPPAG SPAGRSIYNS AYVYCKGPCQ RVQPGKLRVQ
201 CSTCRQATLT LTQGPSCWDD VLIPNRMSGE CQSPHCPGTS
241 AEFFFKCGAH PTSDKETSVA LHLIATNSRN ITCITCTDVR
281 SPVLVFQCNS RHVICLDCFH LYCVTRLNDR QFVHDPQLGY
321 SLPCVAGCPN SLIKELHHFR ILGEEQYNRY QQYGAEECVL
361 QMGGVLCPRP GCGAGLLPEP DQRKVTCEGG NGLGCGFAFC
401 RECKEAYHEG ECSAVFEASG TTTQAYRVDE RAAEQARAEA
441 ASKETIKKTT KPCPRCHVPV EKNGGCMHMK CPQPQCRLEW
481 CWNCGCEWNR VCMGDHWFDV (SEQ ID NO: 75).

21. The polynucleotide of any one of claims 1-20, wherein the fusion protein comprises an F146A substitution relative to a reference human Parkin protein sequence of SEQ ID NO: 1.

22. The polynucleotide of any one of claims 1-21, wherein the fusion protein comprises a W403A substitution relative to a reference human Parkin protein sequence of SEQ ID NO: 1.

23. The polynucleotide of any one of claims 1-22, wherein the fusion protein comprises an F463A substitution relative to a reference human Parkin protein sequence of SEQ ID NO: 1.

24. The polynucleotide of any one of claims 1-23, wherein the fusion protein comprises a C457S substitution relative to a reference human Parkin protein sequence of SEQ ID NO: 1.

25. The polynucleotide of claim 22, wherein the fusion protein comprises both an F146A substitution and a W403A substitution relative to a reference human Parkin protein sequence of SEQ ID NO: 1.

26. The polynucleotide of any one of claims 1-25, wherein the fusion protein comprises a F104M substitution relative to a reference human PINK1 protein sequence of SEQ ID NO: 64.

27. The polynucleotide of any one of claims 1-26, wherein the fusion protein comprises both an F146A substitution and a W403A substitution relative to a reference human Parkin protein sequence of SEQ ID NO: 1, and wherein the fusion protein comprises a F104M substitution relative to a reference human PINK1 protein sequence of SEQ ID NO: 64.

28. The polynucleotide of claim 1, wherein the fusion protein comprises a polypeptide sequence at least 95% identical to the sequence of SEQ ID NO: 97 or 98 and comprises two or more amino acid substitutions selected from F104M, W403A, and F463A,
wherein F104M is relative to a reference human PINK1 protein sequence of SEQ ID NO: 64, W403A is relative to a reference human Parkin protein sequence of SEQ ID NO: 1, and F463A is relative to a reference human Parkin protein sequence of SEQ ID NO: 1.

29. The polynucleotide of claim 1, wherein the fusion protein comprises a polypeptide sequence identical to the sequence any one of SEQ ID NO: 97 or 98 and comprises two or more amino acid substitutions selected from F104M, W403A, and F463A,
wherein F104M is relative to a reference human PINK1 protein sequence of SEQ ID NO: 64, W403A is relative to a reference human Parkin protein sequence of SEQ ID NO: 1, and F463A is relative to a reference human Parkin protein sequence of SEQ ID NO: 1.

30. A vector, comprising the polynucleotide of any one of claims 1-29.

31. The vector of claim 30, wherein the vector is an adeno-associated virus (AAV) vector.

32. The vector of claim 31, wherein the vector comprises an AAV9 capsid or functional variant thereof.

33. The vector of claim 32, wherein the AAV9 capsid shares at least 98%, 99%, or 100% identity to a reference AAV9 capsid.

34. A method of increasing Parkin activity in a cell, comprising contacting the cell with the polynucleotide of any one of claims 1-29 or the vector of any one of claims 30-33.

35. A method of increasing Parkin activity in a subject, comprising administering to the subject the polynucleotide of any one of claims 1-29 or the vector of any one of claims 30-33.

36. The method of claim 34 or claim 35, wherein the cell or subject is deficient in Parkin activity and/or comprises a loss-of-function mutation in Parkin.

37. The method of any one of claims 34-36, wherein Parkin activity comprises one or more of colocalization of Parkin with TOMM2 in response to neurotoxin treatment, ubiquitination of mitochondrial proteins in response to neurotoxin treatment, and increased in Parkin levels in the mitochondrial fraction in response to neurotoxin treatment.

38. A method of promoting survival of a neuron, comprising contacting the neuron with a polynucleotide of any one of claims 1-29 or the vector of any one of claims 30-33.

39. A method of promoting survival of a neuron in a subject, comprising administering to the subject the polynucleotide of any one of claims 1-29 or the vector of any one of claims 30-33.

40. The method of claim 38 or claim 39, wherein the neuron is a dopaminergic neuron.

41. A method of treating a disease or disorder in a subject in need thereof, comprising administering to the subject the polynucleotide of any one of claims 1-29 or the vector of any one of claims 30-33.

42. The method of claim 41, wherein the subject suffers from a genetic deficiency in Parkin expression or function.

43. The method of claim 41 or claim 42, wherein the subject suffers from a genetic deficiency in PINK1 expression or function.

44. The method of any one of claims 41-43, wherein the disease or disorder is Parkinson’s disease.

45. The method of claim 44, wherein the Parkinson’s disease is early onset Parkinson’s disease (EOPD).

46. The method of any one of claims 41-45, wherein the method alleviates one or more symptoms of Parkinson’s disease.

47. The method of any one of claims 41-46, wherein the method reduces motor complications associated with neurodegeneration; reduces the need for antiparkinsonian pharmacotherapy, optionally L-DOPA and/or dopaminergic agonists; restores the function of degenerating neurons; and/or protects neurons from degeneration.

48. The method of any one of claims 41-47, wherein the method enhances nigrostriatal function, optionally assessed by [18F]fluoro-L-dopa positron emission tomography (PET) or DaT-SPECT imaging.

49. The method of any one of claims 41-48, wherein the method improves one or both of the UPDRS or MDS-UPDRS of the subject.

50. A cell comprising the polynucleotide of any one of claims 1-29.

51. A protein encoded by the polynucleotide of any one of claims 1-29.

52. A pharmaceutical composition comprising the vector of any one of claims 30-33 and one or more pharmaceutically acceptable carriers, diluents, or excipients.

53. A kit comprising the vector of any one of claims 30-33 and instructions for use.

54. A recombinant adeno-associated virus (rAAV) virion, comprising a capsid and a vector genome, wherein the vector genome comprises a polynucleotide sequence encoding an activated Parkin protein operatively linked to a promoter.

55. The rAAV virion of claim 55, wherein the activated Parkin protein comprises one or more amino acid substitutions at positions Phe-146, Trp-403, Cys-457, Phe-463, and Asn-273 relative to a reference Parkin protein.

56. The rAAV virion of claim 55, wherein the activated Parkin protein comprises two or more amino acid substitutions at positions Phe-146, Trp-403, Cys-457, Phe-463, and Asn-273 relative to a reference Parkin protein.

57. The rAAV virion of claim 56, wherein the activated Parkin protein comprises amino acid substitutions at positions Phe-146, Trp-403, Cys-457, Phe-463, and Asn-273 relative to a reference Parkin protein.

58. The rAAV virion of any one of claims 54-57, wherein the activated Parkin protein comprises one or more amino acid substitutions selected from F146A, W403A, and/or N273K relative to a reference Parkin protein.

59. The rAAV virion of claim 58, wherein the activated Parkin protein comprises amino acid substitutions F146A and W403A relative to a reference Parkin protein.

60. The rAAV virion of claim 59, wherein the activated Parkin protein comprises amino acid substitutions F146A, N273K, and W403A relative to a reference Parkin protein.

61. The rAAV virion of claim 60, the activated Parkin protein comprises a polypeptide sequence at least 95% identical to human Parkin N273K+W403A+F463A (SEQ ID NO: 93).

62. The rAAV virion of claim 54, wherein the activated Parkin protein comprises a polypeptide sequence identical to human Parkin N273K+W403A+F463A (SEQ ID NO: 93).

63. The rAAV virion of any one of claims 54-60, wherein the Parkin protein is a ΔParkin protein comprising a deletion of the ubiquitin-like (Ubl) domain.

64. The rAAV virion of claim 63, wherein the ΔParkin protein comprises a polypeptide sequence at least 95% identical to residues 76-465 of human Parkin F146A+W403A:
41 KGQEM
81 NATGGDDPRN AAGGCEREPQ SLTRVDLSSS VLPGDSVGLA
121 VILHTDSRKD SPPAGSPAGR SIYNSAYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARAEAASKET IKKTTKPCPR CHVPVEKNGG CMHMKCPQPQ
441 CRLEWCWNCG CEWNRVCMGD HWFDV (SEQ ID NO: 18).

65. The rAAV virion of claim 63, wherein the ΔParkin protein comprises a polypeptide sequence identical to residues 76-465 of human Parkin F146A+W403A:
41 KGQEM
81 NATGGDDPRN AAGGCEREPQ SLTRVDLSSS VLPGDSVGLA
121 VILHTDSRKD SPPAGSPAGR SIYNSAYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARAEAASKET IKKTTKPCPR CHVPVEKNGG CMHMKCPQPQ
441 CRLEWCWNCG CEWNRVCMGD HWFDV (SEQ ID NO: 18).

66. The rAAV virion of any one of claims 54-65, wherein the activated Parkin protein comprises amino acid substitutions at position Cys-431 relative to a reference Parkin protein.

67. The rAAV virion of claim 66, wherein the activated Parkin protein comprises a C431F amino acid substitution relative to a reference Parkin protein.

68. The rAAV virion of any one of claims 54-67, wherein the promoter is a constitutive promoter.

69. The rAAV virion of any one of claims 54-68, wherein the promoter is a CAG promoter.

70. The rAAV virion of any one of claims 54-68, wherein the promoter is a CMV promoter.

71. The rAAV virion of any one of claims 54-67, wherein the promoter is a neuron-specific promoter.

72. The rAAV virion of any one of claims 54-67 or 71, wherein the promoter is a SYN promoter.

73. The rAAV virion of any one of claims 54-72, wherein the vector genome comprises a WPRE element.

74. The rAAV virion of any one of claims 54-73, wherein the vector genome comprises a hGH polyadenylation site.

75. The rAAV virion of any one of claims 54-74, wherein the capsid is an AAV9 capsid or functional variant thereof.

76. The rAAV virion of claim 75, wherein the AAV9 capsid shares at least 98%, 99%, or 100% identity to a reference AAV9 capsid.

77. A method of increasing Parkin activity in a cell, comprising contacting the cell with the rAAV virion of any one of claims 54-76.

78. A method of increasing Parkin activity in a subject, comprising administering to the subject an effective amount of the rAAV virion of any one of claims 54-76.

79. The method of claim 77 or claims 78, wherein the cell or subject is deficient in Parkin activity and/or comprises a loss-of-function mutation in Parkin.

80. The method of any one of claims 77-79, wherein Parkin activity comprises one or more of colocalization of Parkin with TOMM2 in response to neurotoxin treatment, ubiquitination of mitochondrial proteins in response to neurotoxin treatment, and increased in Parkin levels in the mitochondrial fraction in response to neurotoxin treatment.

81. A method of promoting survival of a neuron, comprising contacting the neuron with the rAAV virion of any one of claims 54-76.

82. A method of promoting survival of a neuron in a subject, comprising administering to the subject an effective amount of the rAAV virion of any one of claims 54-76.

83. The method of claim 81 or claim 82, wherein the neuron is a dopaminergic neuron.

84. A method of treating a disease or disorder in a subject in need thereof, comprising administering to the subject an effective amount of the rAAV virion of any one of claims 54-76.

85. The method of claim 84, wherein the subject suffers from a genetic deficiency in Parkin.

86. The method of claim 84, wherein the subject suffers from a genetic deficiency in PINK1.

87. The method of claim 84, wherein the subject suffers from a genetic deficiency in DJ-1.

88. The method of any one of claims 84-87, wherein the disease or disorder is Parkinson’s disease.

89. The method of claim 88, wherein the Parkinson’s disease is early onset Parkinson’s disease (EOPD).

90. The method of any one of claims 84-89, wherein the method alleviates one or more symptoms of Parkinson’s disease.

91. The method of any one of claims 84-90, wherein the method reduces motor complications associated with neurodegeneration; reduces the need for antiparkinsonian pharmacotherapy, optionally L-DOPA and/or dopaminergic agonists; restores the function of degenerating neurons; and/or protects neurons from degeneration.

92. The method of any one of claims 84-91, wherein the method enhances nigrostriatal function, optionally assessed by [18F]fluoro-L-dopa positron emission tomography (PET) or DaT-SPECT imaging.

93. The method of any one of claims 84-92, wherein the method improves one or both of the UPDRS or MDS-UPDRS of the subject.

94. A pharmaceutical composition comprising the rAAV virion of any one of claims 54-76 and one or more pharmaceutically acceptable carriers, diluents, or excipients.

95. A kit comprising the rAAV virion of any one of claims 54-76 and instructions for use.

96. A polynucleotide, comprising a polynucleotide sequence encoding an activated Parkin protein.

97. The polynucleotide of claim 96, wherein the activated Parkin protein comprises amino acid substitutions at position Cys-431 relative to a reference Parkin protein.

98. The polynucleotide of claim 96, wherein the activated Parkin protein comprises a C431F amino acid substitution relative to a reference Parkin protein.

99. The polynucleotide of any one of claims 96-98, wherein the activated Parkin protein comprises one or more amino acid substitutions at positions Phe-146, Trp-403, Cys-457, Phe-463, and Asn-273 relative to a reference Parkin protein.

100. The polynucleotide of claim 99, wherein the activated Parkin protein comprises two or more amino acid substitutions at positions Phe-146, Trp-403, Cys-457, Phe-463, and Asn-273 relative to a reference Parkin protein.

101. The polynucleotide of claim 100, wherein the activated Parkin protein comprises amino acid substitutions at positions Phe-146, Trp-403, Cys-457, Phe-463, and Asn-273 relative to a reference Parkin protein.

102. The polynucleotide of any one of claims 96-101, wherein the activated Parkin protein comprises one or more amino acid substitutions selected from F146A, W403A, and/or N273K relative to a reference Parkin protein.

103. The polynucleotide of claim 102, wherein the activated Parkin protein comprises amino acid substitutions F146A and W403A relative to a reference Parkin protein.

104. The polynucleotide of claim 103, wherein the activated Parkin protein comprises amino acid substitutions F146A, N273K, and W403A relative to a reference Parkin protein.

105. The polynucleotide of claim 96, wherein the activated Parkin protein comprises a polypeptide sequence at least 95% identical to human Parkin N273K+W403A+F463A (SEQ ID NO: 93).

106. The polynucleotide of claim 96, wherein the activated Parkin protein comprises a polypeptide sequence identical to human Parkin N273K+W403A+F463A (SEQ ID NO: 93).

107. The polynucleotide of any one of claims 96-104, wherein the Parkin protein is a ΔParkin protein comprising a deletion of the ubiquitin-like (Ubl) domain.

108. The polynucleotide of any one of claim 107, wherein the ΔParkin protein comprises a polypeptide sequence at least 95% identical to residues 76-465 of human Parkin F146A+W403A:
41 KGQEM
81 NATGGDDPRN AAGGCEREPQ SLTRVDLSSS VLPGDSVGLA
121 VILHTDSRKD SPPAGSPAGR SIYNSAYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARAEAASKET IKKTTKPCPR CHVPVEKNGG CMHMKCPQPQ
441 CRLEWCWNCG CEWNRVCMGD HWFDV (SEQ ID NO: 18).

109. The polynucleotide of claim 108, wherein the ΔParkin protein comprises a polypeptide sequence identical to residues 76-465 of human Parkin F146A+W403A:
41 KGQEM
81 NATGGDDPRN AAGGCEREPQ SLTRVDLSSS VLPGDSVGLA
121 VILHTDSRKD SPPAGSPAGR SIYNSAYVYC KGPCQRVQPG
161 KLRVQCSTCR QATLTLTQGP SCWDDVLIPN RMSGECQSPH
201 CPGTSAEFFF KCGAHPTSDK ETSVALHLIA TNSRNITCIT
241 CTDVRSPVLV FQCNSRHVIC LDCFHLYCVT RLNDRQFVHD
281 PQLGYSLPCV AGCPNSLIKE LHHFRILGEE QYNRYQQYGA
321 EECVLQMGGV LCPRPGCGAG LLPEPDQRKV TCEGGNGLGC
361 GFAFCRECKE AYHEGECSAV FEASGTTTQA YRVDERAAEQ
401 ARAEAASKET IKKTTKPCPR CHVPVEKNGG CMHMKCPQPQ
441 CRLEWCWNCG CEWNRVCMGD HWFDV (SEQ ID NO: 18).

110. The polynucleotide of any one of claims 96-109, wherein the polynucleotide comprises a promoter operably linked to the polynucleotide sequence encoding an activated Parkin protein.

111. The polynucleotide of claim 110, wherein the promoter is a constitutive promoter.

112. The polynucleotide of claim 111, wherein the promoter is a CAG promoter or a CMV promoter.

113. The polynucleotide of claim 110, wherein the promoter is a neuron-specific promoter.

114. The polynucleotide of claim 110 or claim 113, wherein the promoter is a SYN promoter.

115. The polynucleotide of any one of claims 96-114, wherein the polynucleotide comprises a WPRE element.

116. The polynucleotide of any one of claims 96-115, wherein the polynucleotide comprises a hGH polyadenylation site.

117. A vector, comprising the polynucleotide of any one of claims 96-116.

118. The vector of claim 117, wherein the vector is an adeno-associated virus (AAV) vector.

119. The vector of claim 118, wherein the vector comprises an AAV9 capsid or functional variant thereof.

120. The vector of claim 119, wherein the AAV9 capsid shares at least 98%, 99%, or 100% identity to a reference AAV9 capsid.

121. A method of increasing Parkin activity in a cell, comprising contacting the cell with the polynucleotide of any one of claims 96-116 or the vector of any one of claims 117-120.

122. A method of increasing Parkin activity in a subject, comprising administering to the subject the polynucleotide of any one of claims 96-116 or the vector of any one of claims 117-120.

123. The method of claim 121 or claim 122, wherein the cell or subject is deficient in Parkin activity and/or comprises a loss-of-function mutation in Parkin.

124. The method of any one of claims 121-123, wherein Parkin activity comprises one or more of colocalization of Parkin with TOMM2 in response to neurotoxin treatment, ubiquitination of mitochondrial proteins in response to neurotoxin treatment, and increased in Parkin levels in the mitochondrial fraction in response to neurotoxin treatment.

125. A method of promoting survival of a neuron, comprising contacting the neuron with a polynucleotide of any one of claims 96-116 or the vector of any one of claims 117-120.

126. A method of promoting survival of a neuron in a subject, comprising administering to the subject the polynucleotide of any one of claims 96-116 or the vector of any one of claims 117-120.

127. The method of claim 125 or claim 126, wherein the neuron is a dopaminergic neuron.

128. A method of treating a disease or disorder in a subject in need thereof, comprising administering to the subject the polynucleotide of any one of claims 96-116 or the vector of any one of claims 117-120.

129. The method of claim 128, wherein the subject suffers from a genetic deficiency in Parkin expression or function.

130. The method of claim 128 or claim 129, wherein the subject suffers from a genetic deficiency in PINK1 expression or function.

131. The method of any one of claims 128-130, wherein the disease or disorder is Parkinson’s disease.

132. The method of claim 131, wherein the Parkinson’s disease is early onset Parkinson’s disease (EOPD).

133. The method of any one of claims 128-132, wherein the method alleviates one or more symptoms of Parkinson’s disease.

134. The method of any one of claims 128-133, wherein the method reduces motor complications associated with neurodegeneration; reduces the need for antiparkinsonian pharmacotherapy, optionally L-DOPA and/or dopaminergic agonists; restores the function of degenerating neurons; and/or protects neurons from degeneration.

135. The method of any one of claims 128-134, wherein the method enhances nigrostriatal function, optionally assessed by [18F]fluoro-L-dopa positron emission tomography (PET) or DaT-SPECT imaging.

136. The method of any one of claims 128-135, wherein the method improves one or both of the UPDRS or MDS-UPDRS of the subject.

137. A cell comprising the polynucleotide of any one of claims 96-116.

138. A protein encoded by the polynucleotide of any one of claims 96-116.

139. A pharmaceutical composition comprising the vector of any one of claims 117-120 and one or more pharmaceutically acceptable carriers, diluents, or excipients.

140. A kit comprising the vector of any one of claims 117-120 and instructions for use.
